(19) United States
(12) Patent Application Publication
(10) Pub. No.: US 2004/0126784 A1
(43) Pub. Date: Jul. 1, 2004

(54) Modulators of cellular proliferation

(75) Inventors: Yasumichi Hitoshi, Brisbane, CA (US); Yonchu Jenkins, Oakland, CA (US); Vadim Markovtsov, Foster City, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Rigel Pharmaceuticals, Incorporated, South San Francisco, CA (US)

(21) Appl. No.: 10/620,052

(22) Filed: Jul. 14, 2003

Related U.S. Application Data
(60) Provisional application No. 60/395,443, filed on Jul. 12, 2002.

(51) Int. Cl. C12Q 1/68; G01N 33/53; G01N 33/567

(52) U.S. Cl. 435/6; 435/7.2

(57) ABSTRACT

The present invention relates to regulation of cellular proliferation. More particularly, the present invention is directed to nucleic acids encoding protein kinase C ζ (PKC-ζ), phospholipase C-β1 (PLC-β1), protein tyrosine kinase 2 (FAK), protein tyrosine kinase 2b (FAK2), casein kinase 2 (CK2), cMET tyrosine kinase (cMET), flap structure specific endonuclease 1 (FEN1), REV1 dCMP transferase (REV1), apurinic/apyrimidinic nuclease 1 (APE1), cyclin dependent kinase 3 (CDK3), PIM1 kinase (PIM1), cell division cycle 7 kinase (CDC7L1), cyclin dependent kinase 7 (CDK7), cytokine inducible kinase (CNK), potentially prenylated protein tyrosine phosphatase (PRL-3), serine threonine kinase 2 (STK2) or (NEK4), cyclin dependent serine threonine kinase (NKIAMRE), or histone acetylase (HBO1), which are involved in modulation of cell cycle arrest. The invention further relates to methods for identifying and using agents, including small molecule chemical compositions, antibodies, peptides, cyclic peptides, nucleic acids, RNAi, antisense nucleic acids, and ribozymes, that modulate cell cycle arrest via modulation of protein kinase C ζ (PKC-ζ), phospholipase C-β1 (PLC-β1), protein tyrosine kinase 2 (FAK), protein tyrosine kinase 2b (FAK2), casein kinase 2 (CK2), cMET tyrosine kinase (cMET), flap structure specific endonuclease 1 (FEN1), REV1 dCMP transferase (REV1), apurinic/apyrimidinic nuclease 1 (APE1), cyclin dependent kinase 3 (CDK3), PIM1 kinase (PIM1), cell division cycle 7 kinase (CDC7L1), cyclin dependent kinase 7 (CDK7), cytokine inducible kinase (CNK), potentially prenylated protein tyrosine phosphatase (PRL-3), serine threonine kinase 2 (STK2) or (NEK4), cyclin dependent serine threonine kinase (NKIAMRE), or histone acetylase (HBO1), as well as to the use of expression profiles and compositions in diagnosis and therapy related to cell cycle regulation and modulation of cellular proliferation, e.g., for treatment of cancer and other diseases of cellular proliferation.

### CROSS-REFERENCES TO RELATED APPLICATIONS

This application claims the benefit of provisional U.S. Application No. 60/395,443, filed Jul. 12, 2002, which is herein incorporated by reference for all purposes.

### STATEMENT AS TO RIGHTS TO INVENTIONS MADE UNDER FEDERALLY SPONSORED RESEARCH AND DEVELOPMENT

Not applicable.

### FIELD OF THE INVENTION

The present invention relates to regulation of cellular proliferation. More particularly, the present invention is directed to nucleic acids encoding protein kinase C ζ (PKC-ζ), phospholipase C-β1 (PLC-β1), protein tyrosine kinase 2 (FAK), protein tyrosine kinase 2b (FAK2), casein kinase 2 (CK2 or CK2α), cMET tyrosine kinase (cMET), flap structure specific endonuclease 1 (FEN1), REV1 dCMP transferase (REV1), apurinic/apyrimidinic nuclease 1 (APE1), cyclin dependent kinase 3 (CDK3), PIM1 kinase (PIM1), cell division cycle 7 kinase (CDC7L1), cyclin dependent kinase 7 (CDK7), cytokine inducible kinase (CNK), potentially prenylated protein tyrosine phosphatase (PRL-3), serine threonine kinase 2 (STK2) or (NEK4), cyclin dependent serine threonine kinase (NKIAMRE), or histone acetylase (HBO1), which are involved in modulation of cell cycle arrest. The invention further relates to methods for identifying and using agents, including small molecule chemical compositions, antibodies, peptides, cyclic peptides, nucleic acids, RNAi, antisense nucleic acids, and ribozymes, that modulate cell cycle arrest via modulation of protein kinase C ζ (PKC-ζ), phospholipase C-β1 (PLC-β1), protein tyrosine kinase 2 (FAK), protein tyrosine kinase 2 b (FAK2), casein kinase 2 (CK2 or CK2α), cMET tyrosine kinase (cMET), flap structure specific endonuclease 1 (FEN1), REV1 dCMP transferase (REV1), apurinic/apyrimidinic nuclease 1 (APE1), cyclin dependent kinase 3 (CDK3), PIM1 kinase (PIM1), cell division cycle 7 kinase (CDC7L1), cyclin dependent kinase 7 (CDK7), cytokine inducible kinase (CNK), potentially prenylated protein tyrosine phosphatase (PRL-3), serine threonine kinase 2 (STK2) or (NEK4), cyclin dependent serine threonine kinase (NKIAMRE), or histone acetylase (HBO1), as well as to the use of expression profiles and compositions in diagnosis and therapy related to cell cycle regulation and modulation of cellular proliferation, e.g., for treatment of cancer and other diseases of cellular proliferation.

### BACKGROUND OF THE INVENTION

Cell cycle regulation plays a critical role in neoplastic disease, as well as disease caused by non-cancerous, pathologically proliferating cells. Normal cell proliferation is tightly regulated by the activation and deactivation of a series of proteins that constitute the cell cycle machinery. The expression and activity of components of the cell cycle can be altered during the development of a variety of human disease such as cancer, cardiovascular disease, psoriasis, where aberrant proliferation contributes to the pathology of the illness. There are genetic screens to isolate important components for cell cycle regulation using different organisms such as yeast, worms, flies, etc. However, involvement of a protein in cell cycle regulation in a model system is not always indicative of its role in cancer and other proliferative disease. Thus, there is a need to establish screening for understanding human diseases caused by disruption of cell cycle regulation. Identifying proteins, their ligands and substrates, and downstream signal transduction pathways involved in cell cycle regulation and neoplasia in humans is important for developing therapeutic regents to treat cancer and other proliferative diseases.

### BRIEF SUMMARY OF THE INVENTION

The present invention therefore provides nucleic acids encoding protein kinase C ζ (PKC-ζ), phospholipase C-β1 (PLC-β1), protein tyrosine kinase 2 (FAK), protein tyrosine kinase 2b (FAK2), casein kinase 2 (CK2 or CK2α), cMET tyrosine kinase (cMET), flap structure specific endonuclease 1 (FEN1), REV1 dCMP transferase (REV1), apurinic/apyrimidinic nuclease 1 (APE1), cyclin dependent kinase 3 (CDK3), PIM1 kinase (PIM1), cell division cycle 7 kinase (CDC7L1), cyclin dependent kinase 7 (CDK7), cytokine inducible kinase (CNK), potentially prenylated protein tyrosine phosphatase (PRL-3), serine threonine kinase 2 (STK2) or (NEK4), cyclin dependent serine threonine kinase (NKIAMRE), or histone acetylase (HBO1), which are involved in modulation of cell cycle arrest in tumor cells and other pathologically proliferating cells. The invention therefore provides methods of screening for compounds, e.g., small organic molecules, antibodies, peptides, cyclic peptides, nucleic acids, antisense molecules, RNAi, and ribozymes, that are capable of modulating cellular proliferation and/or cell cycle regulation, e.g., either inhibiting cellular proliferation, or activating apoptosis. Therapeutic and diagnostic methods and reagents are also provided. Modulators of protein kinase C ζ (PKC-ζ), phospholipase Cβ1 (PLC-β1), protein tyrosine kinase 2 (FAK), protein tyrosine kinase 2b (FAK2), casein kinase 2 (CK2 or CK2α), cMET tyrosine kinase (cMET), flap structure specific endonuclease 1 (FEN1), REV1 dCMP transferase (REV1), apurinic/apyrimidinic nuclease 1 (APE1), cyclin dependent kinase 3 (CDK3), PIM1 kinase (PIM1), cell division cycle 7 kinase (CDC7L1), cyclin dependent kinase 7 (CDK7), cytokine inducible kinase (CNK), potentially prenylated protein tyrosine phosphatase (PRL-3), serine threonine kinase 2 (STK2) or (NEK4), cyclin dependent serine threonine kinase (NKIAMRE), or histone acetylase (HBO1) are therefore useful in treatment of cancer and other proliferative diseases.

One embodiment of the present invention provides a method for identifying a compound that modulates cell cycle arrest. A cell comprising a protein kinase C ¢ (PKC-ζ), phospholipase C-β1 (PLC-β1), protein tyrosine kinase 2 (FAK), protein tyrosine kinase 2b (FAK2), casein kinase 2 (CK2 or CK2α), cMET tyrosine kinase (cMET), flap structure specific endonuclease 1 (FEN1), REV1 dCMP transferase (REV1), apurinic/apyrimidinic nuclease 1 (APE1), cyclin dependent kinase 3 (CDK3), PIM1 kinase (PIM1), cell division cycle 7 kinase (CDC7L1), cyclin dependent kinase 7 (CDK7), cytokine inducible kinase (CNK), potentially prenylated protein tyrosine phosphatase (PRL-3), serine threonine kinase 2 (STK2) or (NEK4), cyclin dependent serine threonine kinase (NKIAMRE), or histone acetylase (HBO1) polypeptide or fragment thereof is contacted with the compound. The protein kinase C ζ (PKC-ζ), phospholipase C-β1 (PLC-β1), protein tyrosine kinase 2 (FAK), protein tyrosine kinase 2b (FAK2), casein kinase 2 (CK2 or CK2α), cMET tyrosine kinase (cMET), flap structure specific endonuclease 1 (FEN1), REV1 dCMP transferase (REV1), apurinic/apyrimidinic nuclease 1 (APE1), cyclin dependent kinase 3 (CDK3), PIM1 kinase (PIM1), cell division cycle 7 kinase (CDC7L1), cyclin dependent kinase 7 (CDK7), cytokine inducible kinase (CNK), potentially prenylated protein tyrosine phosphatase (PRL-3), serine threonine kinase 2 (STK2) or (NEK4), cyclin dependent serine threonine kinase (NKIAMRE), or histone acetylase (HBO1) polypeptide or fragment thereof may be encoded by a nucleic acid that hybridizes under stringent conditions to a nucleic acid encoding a polypeptide having an amino acid sequence of SEQ ID NO:2, 4, 6, 8, 10, 12, 14, 16, 18, 20, 22, 24, 26, 28, 30, 32, 34, or 36. The chemical or phenotypic effect of the compound upon the cell comprising the protein kinase C ζ (PKC-ζ), phospholipase C-β1 (PLC-1), protein tyrosine kinase 2 (FAK), protein tyrosine kinase 2b (FAK2), casein kinase 2 (CK2 or CK2α), cMET tyrosine kinase (cMET), flap structure specific endonuclease 1 (FEN1), REV1 dCMP transferase (REV1), apurinic/apyrimidinic nuclease 1 (APE1), cyclin dependent kinase 3 (CDK3), PIM1 kinase (PIM1), cell division cycle 7 kinase (CDC7L1), cyclin dependent kinase 7 (CDK7), cytokine inducible kinase (CNK), potentially prenylated protein tyrosine phosphatase (PRL-3), serine threonine kinase 2 (STK2) or (NEK4), cyclin dependent serine threonine kinase (NKIAMRE), or histone acetylase (HBO1) polypeptide or fragment thereof is determined, thereby identifying a compound that modulates cell cycle arrest. The chemical or phenotypic effect may be determined by measuring enzymatic activity of the protein kinase C ζ (PKC-ζ), phospholipase C-β1 (PLC-β1), protein tyrosine kinase 2 (FAK), protein tyrosine kinase 2b (FAK2), casein kinase 2 (CK2 or CK2α), cMET tyrosine kinase (cMET), flap structure specific endonuclease 1 (FEN1), REV1 dCMP transferase (REV1), apurinic/apyrimidinic nuclease 1 (APE1), cyclin dependent kinase 3 (CDK3), PIM1 kinase (PIM1), cell division cycle 7 kinase (CDC7L1), cyclin dependent kinase 7 (CDK7), cytokine inducible kinase (CNK), potentially prenylated protein tyrosine phosphatase (PRL-3), serine threonine kinase 2 (STK2) or (NEK4), cyclin dependent serine threonine kinase (NKIAMRE), or histone acetylase (HBO1) polypeptide. The chemical or phenotypic effect may be determined by measuring cell cycle arrest. The cell cycle arrest may be measured by assaying DNA synthesis or fluorescent marker level. DNA synthesis may be measured by 3H thymidine incorporation, BrdU incorporation, or Hoescht staining. The fluorescent marker may be a cell tracker dye or green fluorescent protein. Modulation may be activation of cell cycle arrest or activation of cancer cell cycle arrest. The host cell may be a cancer cell. The cancer cell may be a breast, prostate, colon, or lung cancer cell. The cancer cell may be a transformed cell line, such as, for example, PC3, H1299, MDA-MB-231, MCF7, A549, or HeLa. The cancer cell may be p53 null, p53 mutant, or p53 wild-type. The polypeptide may recombinant. The polypeptide may be encoded by a nucleic acid comprising a sequence of SEQ ID NO:1, 3, 5, 7, 9, 11, 13, 15, 17, 19, 21, 23, 25, 27, 29, 31, 33, or 35. The compound may be an antibody, an antisense molecule, a small organic molecule, a peptide, a circular peptide, or an siRNA molecule.

Another embodiment of the invention provides a method for identifying a compound that modulates cell cycle arrest. The compound is contacted with a protein kinase C ζ(PKC-ζ), phospholipase C-β1 (PLC-β1), protein tyrosine kinase 2 (FAK), protein tyrosine kinase 2b (FAK2), casein kinase 2 (CK2 or CK2α), cMET tyrosine kinase (cMET), flap structure specific endonuclease 1 (FEN1), REV1 dCMP transferase (REV1), apurinic/apyrimidinic nuclease1 (APE1), cyclin dependent kinase 3 (CDK3), PIM1 kinase (PIM1), cell division cycle 7 kinase (CDC7L1), cyclin dependent kinase 7 (CDK7), cytokine inducible kinase (CNK), potentially prenylated protein tyrosine phosphatase (PRL-3), serine threonine kinase 2 (STK2) or (NEK4), cyclin dependent serine threonine kinase (NKIAMRE), or histone acetylase (HBO1) polypeptide or fragment thereof. The protein kinase C ζ (PKC-ζ), phospholipase C-β1 (PLC-β1), protein tyrosine kinase 2 (FAK), protein tyrosine kinase 2b (FAK2), casein kinase 2 (CK2 or CK2α), cMET tyrosine kinase (cMET), flap structure specific endonuclease 1 (FEN1), REV1 dCMP transferase (REV1), apurinic/apyrimidinic nuclease 1 (APE1), cyclin dependent kinase 3 (CDK3), PIM1 kinase (PIM1), cell division cycle 7 kinase (CDC7L1), cyclin dependent kinase 7 (CDK7), cytokine inducible kinase (CNK), potentially prenylated protein tyrosine phosphatase (PRL-3), serine threonine kinase 2 (STK2) or (NEK4), cyclin dependent serine threonine kinase (NKIAMRE), or histone acetylase (HBO1) polypeptide or a fragment thereof may be encoded by a nucleic acid that hybridizes under stringent conditions to a nucleic acid encoded by a polypeptide comprising an amino acid sequence of SEQ ID NO:2, 4, 6, 8, 10, 12, 14, 16, 18, 20, 22, 24, 26, 28, 30, 32, 34, or 36. The physical effect of the compound upon the protein kinase C ζ (PKC-ζ, phospholipase C-β1 (PLC-β1), protein tyrosine kinase 2 (FAK), protein tyrosine kinase 2b (FAK2), casein kinase 2 (CK2), cMET tyrosine kinase (cMET), flap structure specific endonuclease 1 (FEN1), REV1 dCMP transferase (REV1), apurinic/apyrimidinic nuclease 1 (APE1), cyclin dependent kinase 3 (CDK3), PIM1 kinase (PIM1), cell division cycle 7 kinase (CDC7L1), cyclin dependent kinase 7 (CDK7), cytokine inducible kinase (CNK), potentially prenylated protein tyrosine phosphatase (PRL-3), serine threonine kinase 2 (STK2) or (NEK4), cyclin dependent serine threonine kinase (NKIAMRE), or histone acetylase (HBO1) polypeptide is determined. The chemical or phenotypic effect of the compound upon a cell comprising a protein kinase C ζ (PKC-ζ), phospholipase C-β1 (PLC-β1), protein tyrosine kinase 2 (FAK), protein tyrosine kinase 2b (FAK2), casein kinase 2 (CK2 or CK2α), cMET tyrosine kinase (cMET), flap structure specific endonuclease 1 (FEN1), REV1 dCMP transferase (REV1), apurinic/apyrimidinic nuclease 1 (APE1), cyclin dependent kinase 3 (CDK3), PIM1 kinase (PIM1), cell division cycle 7 kinase (CDC7L1), cyclin dependent kinase 7 (CDK7), cytokine inducible kinase (CNK), potentially prenylated protein tyrosine phosphatase (PRL-3), serine threonine kinase 2 (STK2) or (NEK4), cyclin dependent serine threonine kinase (NKIAMRE), or histone acetylase (HBO1) polypeptide or fragment thereof is determined, thereby identifying a compound that modulates cell cycle arrest.

Yet another embodiment of the invention provides a method of modulating cell cycle arrest in a subject. A therapeutically effective amount of a compound identified according to one of the methods described above is administered to the subject. The subject may be a human. The subject may have cancer. The compound may inhibit cancer cell proliferation.

Even another embodiment of the invention provides a method of modulating cell cycle arrest in a subject. A therapeutically effective amount of a protein kinase C ζ (PKC-ζ), phospholipase C-β1 (PLC-β1), protein tyrosine kinase 2 (FAK), protein tyrosine kinase 2b (FAK2), casein kinase 2 (CK2), cMET tyrosine kinase (cMET), flap structure specific endonuclease 1 (FEN1), REV1 dCMP transferase (REV1), apurinic/apyrimidinic nuclease 1 (APE1), cyclin dependent kinase 3 (CDK3), PIM1 kinase (PIM1), cell division cycle 7 kinase (CDC7L1), cyclin dependent kinase 7 (CDK7), cytokine inducible kinase (CNK), potentially prenylated protein tyrosine phosphatase (PRL-3), serine threonine kinase 2 (STK2) or (NEK4), cyclin dependent serine threonine kinase (NKIAMRE), or histone acetylase (HBO1) polypeptide is administered to the subject. The protein kinase C ζ (PKC-ζ), phospholipase C-β1 (PLC-β1), protein tyrosine kinase 2 (FAK), protein tyrosine kinase 2b (FAK2), casein kinase 2 (CK2), cMET tyrosine kinase (cMET), flap structure specific endonuclease 1 (FEN1), REV1 dCMP transferase (REV1), apurinic/apyrimidinic nuclease 1 (APE1), cyclin dependent kinase 3 (CDK3), PIM1 kinase (PIM1), cell division cycle 7 kinase (CDC7L1), cyclin dependent kinase 7 (CDK7), cytokine inducible kinase (CNK), potentially prenylated protein tyrosine phosphatase (PRL-3), serine threonine kinase 2 (STK2) or (NEK4), cyclin dependent serine threonine kinase (NKIAMRE), or histone acetylase (HBO1) polypeptide may be encoded by a nucleic acid that hybridizes under stringent conditions to a nucleic acid encoding a polypeptide having an amino acid sequence of SEQ ID NO:2, 4, 6, 8, 10, 12, 14, 16, 18, 20, 22, 24, 26, 28, 30, 32, 34, or 36.

A further embodiment of the invention provides a method of modulating cell cycle arrest in a subject. A therapeutically effective amount of anucleic acid encoding a protein kinase C ζ (PKC-ζ), phospholipase C-β1 (PLC-β1), protein tyrosine kinase 2 (FAK), protein tyrosine kinase 2b (FAK2), casein kinase 2 (CK2), cMET tyrosine kinase (cMET), flap structure specific endonuclease 1 (FEN1), REV1 dCMP transferase (REV1), apurinic/apyrimidinic nuclease 1 (APE1), cyclin dependent kinase 3 (CDK3), PIM1 kinase (PIM1), cell division cycle 7 kinase (CDC7L1), cyclin dependent kinase 7 (CDK7), cytokine inducible kinase (CNK), potentially prenylated protein tyrosine phosphatase (PRL-3), serine threonine kinase 2 (STK2) or (NEK4), cyclin dependent serine threonine kinase (NKIAMRE), or histone acetylase (HBO1) polypeptide is administered to the subject. The protein kinase C ζ (PKC-ζ), phospholipase C-β1 (PLC-β1), protein tyrosine kinase 2 (FAK), protein tyrosine kinase 2b (FAK2), casein kinase 2 (CK2), cMET tyrosine kinase (cMET), flap structure specific endonuclease 1 (FEN1), REV1 dCMP transferase (REV1), apurinic/apyrimidinic nuclease 1 (APE1), cyclin dependent kinase 3 (CDK3), PIM1 kinase (PIM1), cell division cycle 7 kinase (CDC7L1), cyclin dependent kinase 7 (CDK7), cytokine inducible kinase (CNK), potentially prenylated protein tyrosine phosphatase (PRL-3), serine threonine kinase 2 (STK2) or (NEK4), cyclin dependent serine threonine kinase (NKIAMRE), or histone acetylase (HBO1) polypeptide may be encoded by a nucleic acid that hybridizes under stringent conditions to a nucleic acid encoding a polypeptide having an amino acid sequence of SEQ ID NO:2, 4, 6, 8, 10, 12, 14, 16, 18, 20, 22, 24, 26, 28, 30, 32, 34, or 36.

The invention also provides specific siRNA molecules for inhibition of expression of cell cycle genes. In one embodiment, the invention provides a CK2-specific siRNA molecule comprising the sequence AACATTGAATTAGATCCACGT. The CK2-specific siRNA molecule can be from 21 to 30 nucleotide base pairs in length. In one aspect, the CK2-specific siRNA molecule has the sequence AACATTGAATTAGATCCACGT and its complement as active portion. The CK2-specific siRNA molecules can be used in a method of inhibiting expression of a CK2 gene in a cell, by contacting the cell with the method comprising contacting the cell with a CK2-specific siRNA molecule from 21 to 30 nucleotide base pairs in length that includes the sequence AACATTGAATTAGATCCACGT.

In another embodiment, the invention provides a PIM1-specific siRNA molecule comprising the sequence AAAACTCCGAGTGAACTGGTC. The PIM1-specific siRNA molecule can be from 21 to 30 nucleotide base pairs in length. In one aspect, the PIM1-specific siRNA molecule has the sequence AAAACTCCGAGTGAACTGGTC and its complement as active portion. The PIM1-specific siRNA molecules can be used in a method of inhibiting expression of a PIM1 gene in a cell, by contacting the cell with the method comprising contacting the cell with a PIM1-specific siRNA molecule from 21 to 30 nucleotide base pairs in length that includes the sequence AAAACTCCGAGTGAACTGGTC.

In another embodiment, the invention provides a Hbo1-specific siRNA molecule comprising the sequence AACTGAGCAAGTGGTTGATTT. The Hbo1-specific siRNA molecule can be from 21 to 30 nucleotide base pairs in length. In one aspect, the Hbo1-specific siRNA molecule has the sequence AACTGAGCAAGTGGTTGATTT and its complement as active portion. The Hbo1-specific siRNA molecules can be used in a method of inhibiting expression of a Hbo1 gene in a cell, by contacting the cell with the method comprising contacting the cell with a Hbo1-specific siRNA molecule from 21 to 30 nucleotide base pairs in length that includes the sequence AACTGAGCAAGTGGTTGATTT.

Other embodiments and advantages of the present invention will be apparent from the detailed description that follows.

### BRIEF DESCRIPTION OF THE DRAWINGS

FIG. 1 provides a nucleotide (SEQ ID NO:1) and amino acid (SEQ ID NO:2) sequence of human PKC-ζ.

FIG. 2 provides a nucleotide (SEQ ID NO:3) and an amino acid (SEQ ID NO:4) sequence of human PLC-β1.

FIG. 3 provides a nucleotide (SEQ ID NO:5) and an amino acid (SEQ ID NO:6) sequence of human FAK.

FIG. 4 provides a nucleotide (SEQ ID NO:7) and an amino acid (SEQ ID NO:8) sequence of human FAK2.

FIG. 5 provides a nucleotide (SEQ ID NO:9) and an amino acid (SEQ ID NO:10) sequence of human CK2.

FIG. 6 provides a nucleotide (SEQ ID NO:11) and an amino acid (SEQ ID NO:12) sequence of human cMET.

FIG. 7 provides a nucleotide (SEQ ID NO:13) and an amino acid (SEQ ID NO:14) sequence of human FEN1.

FIG. 8 provides a nucleotide (SEQ ID NO:15) and an amino acid (SEQ ID NO:16) sequence of human REV1.

FIG. 9 provides a nucleotide (SEQ ID NO:17) and an amino acid (SEQ ID NO:18) sequence of human APE1.

FIG. 10 provides a nucleotide (SEQ ID NO:19) and an amino acid (SEQ ID NO:20) sequence of human CDK3.

FIG. 11 provides a nucleotide (SEQ ID NO:21) and an amino acid (SEQ ID NO:22) sequence of human PIM1.

FIG. 12 provides a nucleotide (SEQ ID NO:23) and an amino acid (SEQ ID NO:24) sequence of human CDC7L1.

FIG. 13 provides a nucleotide (SEQ ID NO:25) and an amino acid (SEQ ID NO:26) sequence of human CDK7.

FIG. 14 provides a nucleotide (SEQ ID NO:27) and an amino acid (SEQ ID NO:28) sequence of human CNK.

FIG. 15 provides a nucleotide (SEQ ID NO:29) and an amino acid (SEQ ID NO:30) sequence of human PRL-3.

FIG. 16 provides a nucleotide (SEQ ID NO:31) and an amino acid (SEQ ID NO:32) sequence of human STK2 (NEK4).

FIG. 17 provides a nucleotide (SEQ ID NO:33) and an amino acid (SEQ ID NO:34) sequence of human NKIAMRE.

FIG. 18 provides a nucleotide (SEQ ID NO:35) and an amino acid (SEQ ID NO:36) sequence of human HBO1.

FIG. 19 provides a table summarizing genes that may be involved in the modulation of cell proliferation.

FIG. 20 illustrates inhibition of proliferation of A549 cells by expression of wild-type GFP-CDC7LI and mutant GFP-CDC7LI.

FIG. 21 illustrates inhibition of proliferation of A549 cells by expression of wild-type CNK and mutant GFP-CNK.

FIG. 22 illustrates inhibition of proliferation of A549 cells and Hela cells by expression of wild-type and mutant STK2.

FIG. 23 provides amino acid sequences for dominant negative mutants of CDC7L1.

FIG. 24 provides amino acid sequences for dominant negative mutants of CNK.

FIG. 25 provides amino acid sequences for dominant negative mutants of STK2.

FIG. 26 provides an alternative view of the amino acid sequences for dominant negative mutants of CDC7L1.

FIG. 27 provides Taqman analysis (i.e., real time PCR) of Cdc7L mRNA expression using RNA from tumor cell lines and primary human cell lines. Cdc7L mRNA levels were normalized to GAPDH mRNA levels.

FIG. 28 provides analysis of CDC7L mRNA levels in matched cancerous and normal tissue from patients with lung carcinoma. Each matched pair represents a different patient.

FIG. 29 provides analysis of CDC7L mRNA in matched cancerous and normal tissue from patients with colon carcinoma. Each matched pair represents a different patient.

FIG. 30 provides Taqman analysis (i.e., real time PCR) of CNK mRNA expression using RNA from tumor cell lines and primary human cell lines. CNK mRNA levels were normalized to GAPDH mRNA levels.

FIG. 31 demonstrates that GST-CNK produced in E. coli has kinase activity against p53 and MBP. GST-CNK also exhibits autophosphorylation activity.

FIG. 32 depicts the structure of STK2 long (STK2L) and short (STK2L) forms and their expression levels in human tissues.

FIG. 33 provides Taqman analysis (ie., real time PCR) of STK2 mRNA expression using RNA from tumor cell lines and primary human cell lines. STK2 mRNA levels were normalized to GAPDH mRNA levels.

FIG. 34 demonstrates that GFP-STK2S expression is antiproliferative when measured using the cell tracker assay.

FIG. 35 demonstrates that GFP-STK2L expression is antiproliferative in A549 and HeLa cells.

FIG. 36 demonstrates that GFP-STK2L expression is antiproliferative when measured using the cell tracker assay.

FIG. 37 demonstrates that IRES-STK2L expression is antiproliferative in A549 and HeLa cells.

FIG. 38 demonstrates that expression of IRES Hbo1 E508Q is antiproliferative in A549 cells.

FIG. 39 demonstrates that no significant differences in proliferation are observed between Hbo1 WT and mutant proteins when expressed in H 1299 cells.

FIG. 40 demonstrates that expression of Hbo1 mtant E508Q is antiproliferative in HeLa cells.

FIG. 41 depicts analysis of proliferation in sorted cells that express wild type or mutant Hbo1 proteins.

FIG. 42 demonstrates that expression of HBO1 mutant E508Q is antiproliferative in sorted A549 cells.

FIG. 43 demonstrates that expression of HBO1 mutant E508Q is antiproliferative in sorted HeLa cells.

FIG. 44 demonstrates that expression of HBO1-specific siRNA reduces Hbo1 mRNA levels and has an antiproliferative effect on A549 cells.

FIG. 45 demonstrates that HBO1-specific siRNA reduces Hbo1 mRNA levels and has an antiproliferative effect on 1299 cells.

FIG. 46 provides Taqman analysis (i.e., real time PCR) of PIM1 mRNA expression using RNA from tumor cell lines and primary human cell lines. PIM1 mRNA levels were normalized to 18S RNA levels.

FIG. 47 provides Taqman analysis (i.e., real time PCR) of PIM1 mRNA levels in matched cancerous and normal tissue from patients with breast carcinoma. Each matched pair represents a different patient. PIM1 mRNA levels were normalized to 18S RNA levels.

FIG. 48 provides Taqman analysis (i.e., real time PCR) of PIM1 mRNA levels in matched cancerous and normal tissue from patients with lung carcinoma. Each matched pair represents a different patient. PIM 1 mRNA levels were normalized to 18S RNA levels.

FIG. 49 demonstrates that expression of PIM1 wild type, but not mutant protein, is antiproliferative in A549 cells.

FIG. 50 demonstrates that expression of GFP-PIM1 wild type is antiproliferative in H1299 cells. The figure also demonstrates that expression of both IRES PIM1 wild type and mutant is antiproliferative in H1299 cells.

FIG. 51 demonstrates that expression of PIM1-specific siRNA reduces PIM1 mRNA levels and has an antiproliferative effect on A549 cells.

FIG. 52 demonstrates that expression of PIM1-specific siRNA reduces PIM1 mRNA levels and has an antiproliferative effect on HeLa cells.

FIG. 53 demonstrates that expression of PIM1-specific siRNA reduces PIM1 mRNA levels and has an antiproliferative effect on H1299 cells.

FIG. 54 demonstrates that expression of PIM1-specific siRNA reduces PIM1 mRNA levels and has an antiproliferative effect on primary HUVEC cells.

FIG. 55 demonstrates that expression of APE1 wild type and mutant proteins is not antiproliferative in A549 cells.

FIG. 56 demonstrates that expression of APE1 wild type and mutant proteins is not antiproliferative in H1299 cells.

FIG. 57 demonstrates that expression of APE1 wild type and APE1 D210A mutant proteins is antiproliferative in primary HMEC cells.

FIG. 58 demonstrates that expression of the Ape1 D210A mutant sensitizes A549 cells to methyl methanesulfonate treatment.

FIG. 59 demonstrates that wild type Ape1 and the Ape1 C65A mutant are protective when expressed in A549 cells treated with bleomycin.

FIG. 60 demonstrates that wild type Ape1 and the Ape1 C65A mutant are protective when expressed in HeLa cells or H1299 cells treated with bleomycin.

FIG. 61 provides Taqman analysis (i.e., real time PCR) of CK2α mRNA expression using RNA from tumor cell lines and primary cell lines. CK2α mRNA levels were normalized to 18S RNA levels.

FIG. 62 provides the sequence of dominant negative mutants of CK2α.

FIG. 63 demonstrates that expression of CK2α-specific siRNA reduces CK2α mRNA levels and has an antiproliferative effect on H1299 cells.

FIG. 64 provides Taqman analysis (i.e., real time PCR) of NKIAMRE expression using RNA from tumor cell lines and primary cell lines. NKIAMRE mRNA levels were normalized to 18S RNA levels.

FIG. 65 provides the sequence of dominant negative mutants of NKIAMRE.

FIG. 66 provides the sequence of dominant negative mutants of FEN1.

FIG. 67 demonstrates that expression of FEN1 dominant negative mutants in A549 cells is antiproliferative.

FIG. 68 demonstrates that expression of FEN1 dominant negative mutants in H1299 cells is antiproliferative.

FIG. 69 provides the sequence of dominant negative mutants of CDK3.

FIG. 70 demonstrates that expression of GFP-CDK3 wild type and CDK3 mutant proteins appears to have no antiproliferative effect in A549 cells. The figure also demonstrates that expression of both IRES CDK3 wild type and CDK3 mutant proteins appears to have no antiproliferative effect in A549 cells.

FIG. 71 demonstrates that expression of GFP-CDK3 wild type and CDK3 mutant proteins appears to have no antiproliferative effect in H1299 cells. The figure also demonstrates that expression of both IRES CDK3 wild type and CDK3 mutant proteins appears to have no antiproliferative effect in H1299 cells.

FIG. 72 provides the sequence of dominant negative mutants of HBO1.

FIG. 73 provides the sequence of dominant negative mutants of PIM1.

FIG. 74 demonstrates that expression of GFP-NKIAMRE wild type and NKIAMRE mutant proteins appears to have no antiproliferative effect in either A549 cells or H1299 cells.

### DETAILED DESCRIPTION OF THE INVENTION

Introduction

PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, and HBO1 encode proteins involved in modulation of the cell cycle in cancer cells.

As described below, the present inventors identified PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, and HBO1 as modulators of the cell cycle in immunoprecipitation assays or yeast 2 hybrid assays.

PKC-ζ encodes an a typical isoform of protein kinase C, i.e., an isoform that is not activated by phorbol esters or diacylglycerols (see, e.g., Donson et al. J. Neuro-Onc., 47:109 (2000)). PKC-ζ activates several signaling pathways, mediates multiple cellular functions, and plays a role in the proliferation of fibroblast cells, endothelial cells, smooth muscle cells, human glioblastoma cells, and astrocytoma cells (see, e.g., Guizzetti and Costa, Biochem. Pharmacol., 60:1457 (2000); Donson et al., 2000). PKC-ζ also plays a role in the activation of p70 S6 kinase which modulates the progression through the G₁ phase of the cell cycle (see, Guizzetti, 2000). Assays known to those of skill in the art can be used to identify modulators of PKC-ζ (see, e.g., J. Biol. Chem., 276:3543; J. Biol. Chem., 272:31130; J. Biol. Chem., 270:15884; J. Biol. Chem., 273:26277; J. Biol. Chem., 272:16578; Mol. Cell. Biol., 19:2180). For example, IRS-1, nucleoli, heterogeneous ribonucleoprotein A1, Sp1, Sendai virus phosphoprotein, and IKK β may be used as substrates in assays to identify modulators of PKCζ (see, e.g., J. Biol. Chem., 276:3543; J. Biol. Chem., 272:31130; J. Biol. Chem., 270:15884; J. Biol. Chem., 273:26277; J. Biol. Chem., 272:16578; Mol. Cell. Biol., 19:2180).

PLC-β1 encodes a phosphoinositide-specific phospholipase C. The PLC-β1 isoform is the predominant nuclear phospholipase C in multiple cell types, including erythroleukemia cells, osteosarcoma cells, pheochromocytoma cells, and glioma cells (see, e.g., Cocco et al., Advan. Enzyme Regul., 39:287 (1999)). PLC-β1 has been shown to be-responsible for nuclear inositol lipid metabolism in multiple cell types (see, e.g., Avazeri, et al., Mol. Biol. Cell, 11:4369 (2000)). Overexpression of PLC-β1 in human colon cancer cells suppresses tumor cell growth, but induces increased cell aggregation and increased expression and release of carcinoembryonic antigen molecule (see, e.g., Nomoto et al., Jpn. J. Canc. Res., 89:1257 (1998)). PLC-β1 has been reported to be essential for IGF-1 induced mitogenesis (see, Cocco et al., 1999). Phospholipase C activity assays known to those of skill in the art can be used to identify modulators of PLC-β1 (see, e.g., Nomoto et al., 1998; Physiol. Rev., 80:1291 (2000); Biochemistry, 36:848; Eur. J. Biochem., 213:339). For example, phosphoinositide may be used as a substrate in assays to identify modulators of PLC-β1 (see, e.g., Nomoto et al., 1998; and Physiol. Rev., 80:1291 (2000); Biochemistry, 36:848; Eur. J. Biochem., 213:339). Additional assays to identify modulators of PLC-β1 are described in, e.g., 109 Mark Dolittle and Karen Reue, Methods in Molecular Biology: Lipase and Phospholipase Protocols (1998)

FAK encodes a cytoplasmic tyrosine kinase that plays a role in regulation of cell cycle progression (see, e.g., MacPhee et al., Lab. Invest., 81(11):1469 (2001) and Zhao et al., Mol. Biol. Cell, 12:4066 (2001)). Specifically, FAK regulates cell cycle progression by increasing cyclin D1 expression and/or decreasing expression of the CDK inhibitor p21 (see, Zhao et al., 2001). High levels of FAK have been linked to tumor invasiveness and metastasis (see, e.g., Fresu et al., Biochem. J., 358:407 (2001)). Tyrosine kinase assays known to those of skill in the art can be used to identify modulators of FAK (see, e.g., Bioessays, 19:137; Mol. Biol. Cell, 10:2507 (1999)). For example, p130Cas and paxillin may be used as a substrate to identify modulators of FAK (see, e.g., Bioessays, 19:137; Mol. Biol. Cell, 10:2507 (1999)).

FAK2 encodes a calcium dependent tyrosine kinase that localizes to sites of cell-to-cell contact and participates in cellular signal transduction (see, e.g., Sasaki et al., J. Bio. Chem., 270(6):21206 (1995) and Li et al., J. Biol. Chem., 273(16):9361 (1998)). Tyrosine kinase assays known to those of skill in the art can be used to identify modulators of FAK2 (see, e.g., Sasaki et al., 1995). For example, p130Cas and paxillin may be used as substrates in assays to identify modulators of FAK2.

CK2 or CK2α encodes an ubiquitous serine threonine protein kinase that is required for the G₂/M transition and checkpoint control stages of the cell cycle (see, e.g. Messenger et al., J. Biol. Chem. 277:23054 (2002), Sayed et al., Oncogene 20(48):6994 (2001), and Escargueil et al. J. Biol. Chem. 275(44):34710 (2000)). In particular, CK2 is required for the phosphorylation of topoisomerase 1 during the G₂/M transition of the cell cycle (see, Messenger et al., 2002). CK2 is overexpressed in tumors and leukemic cells (see, Messenger et al., 2002). CK2 works with p53 in spindle checkpoint arrest to maintain increase cyclin B/cdc2 kinase activity (see, Sayed et al., 2001). Serine threonine protein kinase assays known to those of skill in the art can be used in assays to identify modulators of CK2 (see, e.g., Messenger et al., 2002 and J. Biol. Chem., 274(41):29260).

cMET encodes a tyrosine kinase that is expressed in numerous tissues and plays a role in the generation and spread of tumors of the stomach, rectum, lung, pancreas, breast, and bile duct (see, e.g., Jeffers et al., Proc. Nat'l. Acad. Sci. USA 94:11445 (1997) and Ramirez et al., Endocrinology 53:635 (2000)). More specifically, cMET plays a role in angiogenesis, cell motility, cell growth, cell invasion, and morphogenic differentiation (see, Jeffers et al., 1997). In particular, cMET overexpression is associated with a high risk of metastasis and recurrence of papillary thyroid carcinoma (see, Ramirez et al., 2000). Tyrosine kinase assays known to those of skill in the art can be used in assays to identify modulators of cMET (see, Jeffers et al., 1997). For example dCMP, Grb2, Gab can be used as substrates in assays to identify modulators of cMET.

FEN1 encodes a structure specific endonuclease that cleaves substrates with unannealed 5′ tails (see, e.g., Warbrick et al., J. Pathol. 186:319 (1998)). FEN1 has high specificity of binding/activity toward 5′ flap structures, i.e., dsDNA with a displaced 5′ strand (see, e.g., Warbrick et al., 1998 and Tom et al., J. Biol. Chem. 275(14):10498 (2000)). FEN1 also exhibits a 5′ to 3′ exonucleolytic activity. FEN1 levels are low in non-cycling cells and are induced as the cells enter the cell cycle (see, Warbrick et al., 1998). FEN1 assays known to those of skill in the art can be used to identify modulators of FEN1 (see, Tom et al., 2000 and EMBO J., 13(5):1235 (1994)). For example, 5′ DNA flap structures can be used as substrates in assays to identify modulators of FEN1 (see, e.g., EMBO J., 13(5):1235 (1994)).

REV1 encodes a 1251 amino acid dCMP transferase that functions in the Polζ mutagenesis pathway (see, e.g., Lui et al., Nuc. Acids. Res. 27(22):4468 (1999) and Zhang et al., Nuc. Acids Res. 30(7):1630 (2002)). REV1 has been implicated in UV induced mutagenesis repair and is postulated to play a role in UV damage tolerance (see, e.g., Murakomo, J. Biol. Chem., 276(38):35644 (2001)). dCMP transferase assays known to those of skill in the art can be used to identify modulators of REV1 (see, Zhang et al., 2002 and J. Biol. Chem., 276(18):15051). For example, dCMP, 5′-end 32P-labeled oligonucleotide primer 5′-CACTGACTGTATG-3′ annealed to an oligonucleotide template, 5′-CTCGTCAGCATCTTCAUCATACAGTCAGTG-3′ treated with uracil-DNA glycosylase may be used as substrates in assays to identify modulators of REV1 (see, e.g., J. Biol. Chem., 276(18):15051).

APE1 encodes an apyrimidinic endonuclease that plays a role in short patch repair and long patch repair of ionizing radiation and alkyklating agent induced damage in DNA (see, e.g., Tom et al., J. Biol. Chem., 276(52):48781 (2001), Izumi, Carcinogenesis, 21(7):1329 (2000), and Bobola et al., Clin. Cancer Res. 7(11):3510 (2001)). APE1 has also plays a role the cellular response to oxidative stress, regulation of transcription factors, cell cycle control, and apoptosis (see, Bobola et al., 2001). Assays known to those of skill in the art can be used to identify modulators of APE1 (see, Tom et al., 2001 and Bobola et al., 2001; Nucleic Acids Res., 5(4):1413 (1978); Biochimie, 64(8-9):603 (1982); Mutat. Res., 460(3-4):211 (2000)). For example, oligonucleotide duplexes containing an apurinic/apyrimidinic sites may be used as a substrate in assays to identify modulators of APE1.

CDK3 encodes a cyclin dependent kinase that regulates entry into S phase. (see, e.g., Braun et al., Oncogene, 17(7):2259 (1998)). Specifically, CDK3 has been described as a positive G₁ phase regulator that enhances the G₁/S transition (see, Braun et al., Oncogene, 1998). Overexpression of CDK2 and CDK3 together has been show to elevate c-myc induced apoptosis (see, e.g., Braun et al., DNA Cell Biol., 17(9):789 (1998)). A dominant negative mutant of CDK3 suppresses apoptosis and overexpression of CDK3 circumvents the anti-apoptotic effect of bcl-2 (see, e.g., Meikrantz and Schlegel, J. Biol. Chem., 271(17):10205 (1996)). Assays known to those of skill in the art can be used to identify modulators of CDK3 (see, e.g., Eur. J. Biochem., 268:6076 (2001)). For example, pRb, histone H1, and P701K3-1 (the C-terminal domain of RNA Pol I) may used as substrates in assays to identify modulators of CDK3 (see, e.g., Eur. J. Biochem., 268:6076 (2001)).

PIM1 encodes two cytoplasmic serine threonine kinases generated by an alternate translation initiation (see, e.g., Mochizuki et al., Oncogene 15:1471 (1997) and Shirogane et al., Immunity 11:709 (1999)). PIM1 plays a role in cellular transformation and inhibits apoptosis (see, e.g., Mochizuki et al., 1997). Specifically, PIM1 cooperates with c-myc to promote cell proliferation through the G₁ to S transition and to prevent apoptosis (Shirogane et al., 1999). PIM1 has been implicated in T cell lymphoma, i.e., it has been shown that PIM1 cooperates with the oncoprotein E2α-Pbx1 to facilitate thymic lymphagenesis (see, e.g., Feldman et al., Oncogene 15(22):2735 (1997)). Assays known to those of skill in the art can be used to identify modulators of PIM1 (see, e.g., J. Biol. Chem., 266(21):14018). For example, histone H1 may be used as a substrate in assays to identify modulators of PIM1 (see, e.g., J. Biol. Chem., 266 (21):14018).

CDC7L1 encodes a 574 amino acid serine threonine kinase (see, e.g., Masai and Arai, J. Cell Physiol., 190(3):287 (2002), Masai et al., J. Biol. Chem., 275(37):29042 (2000), and Johnston et al., Prog. Cell Cycle Res., 4:61(2002)). CDC7L1 binds the activator for S phase kinase (ASK) to form a complex that is present at high levels during S phase and decreased levels during G₁ phase. Assays known to those of skill in the art can be used to identify modulators of CDC7L1 (see, e.g., Masai et al., 2000; Johnston et al., 2000; and Proc. Natl. Acad. Sci. USA, 94:14320 (1997)). For example, histone H1 may be used as a substrate in assays to identify modulators of CDC7L1 (see, e.g., Proc. Natl. Acad. Sci. USA, 94:14320 (1997)). Alternatively, Mcm2 may be used as a substrate in assays to identify modulators of CDC7L1 (see, e.g., Takeda et al., Mol. Biol. Cell, 12:1257 (2001)). Conditional muCDC7-deficient embryonic cell lines and transgenic CDC7 knockout mice have been generated (see, e.g., EMBO J. 21L2168 (2002). The cell lines undergo S phase arrest and the knockout mouse is embryonic lethal.

CDK7 encodes a cyclin dependent kinase that is postulated to play a role in cell cycle regulation (see, e.g., Nishiwaki et al., Mol. Cell Biol., 20(20):7726 (2000), Acevedo-Duncan et al., Cell. Prolif. 35(1):23 (2002), and Bregman et al., Front. Biosci., 5:D244 (2000)). CDK7 is the kinase component of the transcription factor complex TFIIH and has been shown to contribute to the ability of p16^{INK4A} to induce cell cycle arrest (see, Nishiwaki et al., 2002). Assays known to those of skill in the art can be used to identify modulators of CDK7 (see, e.g., Mol. Cell. Biol., 21:88 (2001)). For example, CDK2 and the C-terminal domain of RNA Pol II can be used as substrates in assays to identify modulators of CDK7.

CNK is also known as PRK (Proliferation related kinase) and encodes a cytokine inducible serine threonine kinase (see, e.g., Li et al., J. Biol. Chem. 271 (32):19402 (1996), Dai et al., Genes Chromosomes Cancer, 27(3):332 (2000), and Ouyang et al., Oncogene, 18(44):6029 (1999)). CNK is a member of the polo family of kinases which have been implicated in cell division (see, Li et al., 1996). CNK expression is downregulated in lung cancer and in head and neck cancer (see, Li et al., 1996 and Dai et al., 2000). Assays known to those of skill in the art can be used to identify modulators of CNK (see, e.g., J. Biol. Chem., 272:28646). For example, CDC25, p53, and casein can be used as substrates in assays to identify modulators of CNK (see, e.g., J. Biol. Chem., 272:28646).

PRL-3 encodes a 22 kDa potentially prenylated protein tyrosine phosphatase (see, e.g., Zeng et al., Biochem. Biophys. Res. Commun. 244(2):421 (1998), Saha et al., Science, 294(5545):1343 (2001), and Bradbury, Lancet 358(9289):1245 (2001)). PRL-3 is localized to the cytoplasmic membrane when prenylated at its carboxy terminus, and to the nucleus when it is not prenylated (see, Saha et al., 2001). PRL-3 is expressed at low levels in normal colorectal epithelial cells, at intermediate levels in malignant stage I or II cancers, and at high levels in colorectal metastases (see, Saha et al., 2001). Assays known to those of skill in the art can be used to identify modulators of PRL-3.

STK2 is also known as NEK4 and encodes a serine threonine kinase (see, e.g., Chen et al., Gene, 234(1):127 (1999), Hayashi et al., Biochem. Biophys. Res. Commun., 264(2):449 (1999) and Levedakou et al., Oncogene 9(7):1977 (1994). STK2 (NEK4) has been localized to chromosome 3p21.1 and is a member of the NIMA family of kinases which are G₂/M regulators of the cell cycle. Assays known to those of skill in the art can be used to identify modulators of STK2 (NEK4) (see, Hayashi et al., 1999; Biochem. Biophys. Res. Commun. 264(2):449 (1999); J. Biol. Chem. 269:6603 (1994)). For example, the polypeptide FRXT can be used as a substrate in assays to modulate STK2 function.

NKIAMRE encodes the human homologue to the mitogen-activated protein kinase-/cyclin-dependent kinase-related protein kinase NKIATRE (see, e.g., Midermer et al., Cancer Res., 59(16):4069 (1999)). NKIAMRE localizes to chromosome band 5q31 and is deleted in samples from leukemia patients (see, e.g., Midermer et al., 1999). Assays known to those of skill in the art can be used to identify modulators of NKIAMRE.

HBO1 encodes a member of the MYST family of histone acetyltransferases (see, e.g., Iizuka and Stillman, J. Biol. Chem., 274(33):23027 (1999), Sterner and Berger, Microbiol. Mol. Biol. Rev., 64(2):435 (2000), and Burke et al., J. Biol. Chem. 276(18):15397 (2001)). HBO1 binds to ORC (origin recognition complex) to form a complex that plays a role in the initiation of replication (see, Sterner and Berger, 2000). Assays known to those of skill in the art can be used to identify modulators of HBO1 (see, Iizuka and Stillman, 1999 and J. Bio. Chem., 274 (33):23027 (1999)). For example, histone H3 and histone H4 can be used as substrates in assays to identify modulators of HBO1 (see, e.g., J. Bio. Chem., 274(33):23027 (1999)).

Thus, protein kinase C ζ (PKC-ζ), phospholipase C-β1 (PLC-β1), protein tyrosine kinase 2 (FAK), protein tyrosine kinase 2b (FAK2), casein kinase 2 (CK2), cMET tyrosine kinase (cMET), flap structure specific endonuclease 1 (FEN1), REV1 dCMP transferase (REV1), apurinic/apyrimidinic nuclease 1 (APE1), cyclin dependent kinase 3 (CDK3), PIM1 kinase (PIM1), cell division cycle 7 kinase (CDC7L1), cyclin dependent kinase 7 (CDK7), cytokine inducible kinase (CNK), potentially prenylated protein tyrosine phosphatase (PRL-3), serine threonine kinase 2 (STK2) or (NEK4), cyclin dependent serine threonine kinase (NKIAMRE), and histone acetylase (HBO1) can conveniently be used to identify agents that modulate the cell cycle.

PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, and HBO1 therefore represent drug targets for compounds that suppress or activate cellular proliferation in tumor cells, or cause cell cycle arrest, cause release from cell cycle arrest, activate apoptosis, increase sensitivity to chemotherapeutic (adjuvant) reagents, and decrease toxicity of chemotherapeutic reagents. Agents identified in these assays, including small organic molecules, peptides, cyclic peptides, nucleic acids, antibodies, antisense nucleic acids, RNAi, and ribozymes, that modulate cell cycle regulation and cellular proliferation via modulation of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1, can be used to treat diseases related to cellular proliferation, such as cancer. In particular, inhibitors of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 are useful for inhibition of cancer and tumor cell growth. PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 modulators can also be used to modulate the sensitivity of cells to chemotherapeutic agents, such as bleomycin, etoposide, taxol, and other agents known to those of skill in the art. PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 modulators can also be used to decrease toxicity of such chemotherapeutic reagents.

In one embodiment, enzymatic assays, including kinase or autophosphorylation assays, lipase assays, nuclease assays, transferase assays, phosphatase assays, and acetylase assays using PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 can be used to identify modulators of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 activity, or to identify proteins that bind to PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1, e.g., PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 substrates. Full length wild type PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1, mutant PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 can be used in these assays.

Such modulators are useful for treating cancers, such as melanoma, breast, ovarian, lung, gastrointestinal and colon, prostate, and leukemia and lymphomas, e.g., multiple myeloma. In addition, such modulators are useful for treating noncancerous disease states caused by pathologically proliferating cells such as thyroid hyperplasia (Grave's disease), psoriasis, benign prostatic hypertrophy, neurofibromas, atherosclerosis, restenosis, and other vasoproliferative disease.

Definitions

By “disorder associated with cellular proliferation” or “disease associated with cellular proliferation” herein is meant a disease state which is marked by either an excess or a deficit of cellular proliferation or apoptosis. Such disorders associated with increased cellular proliferation include, but are not limited to, cancer and non-cancerous pathological proliferation.

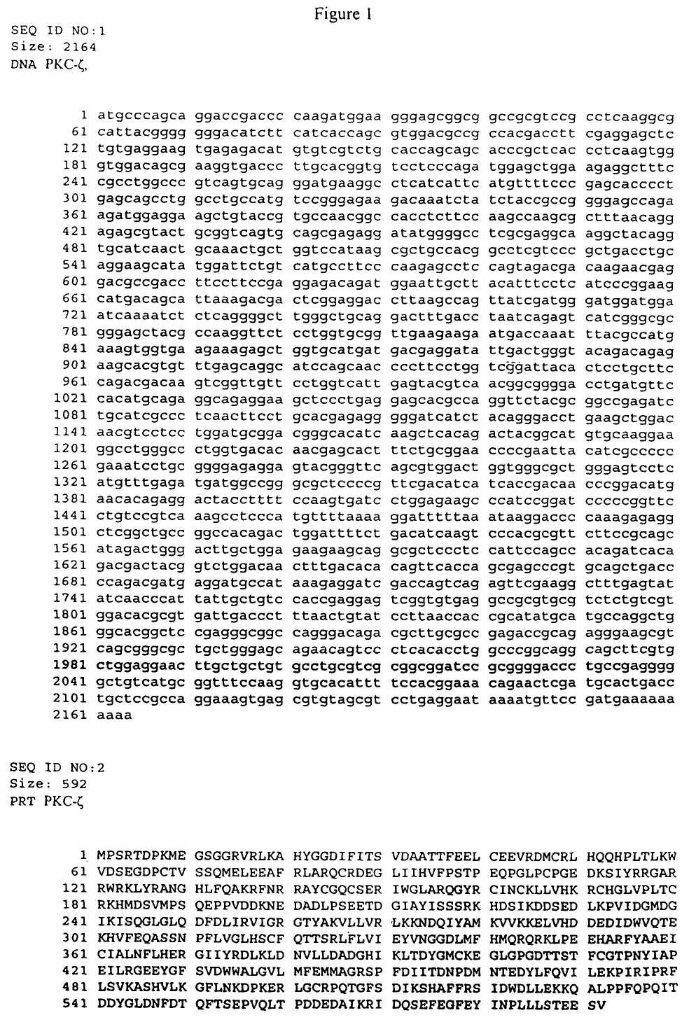

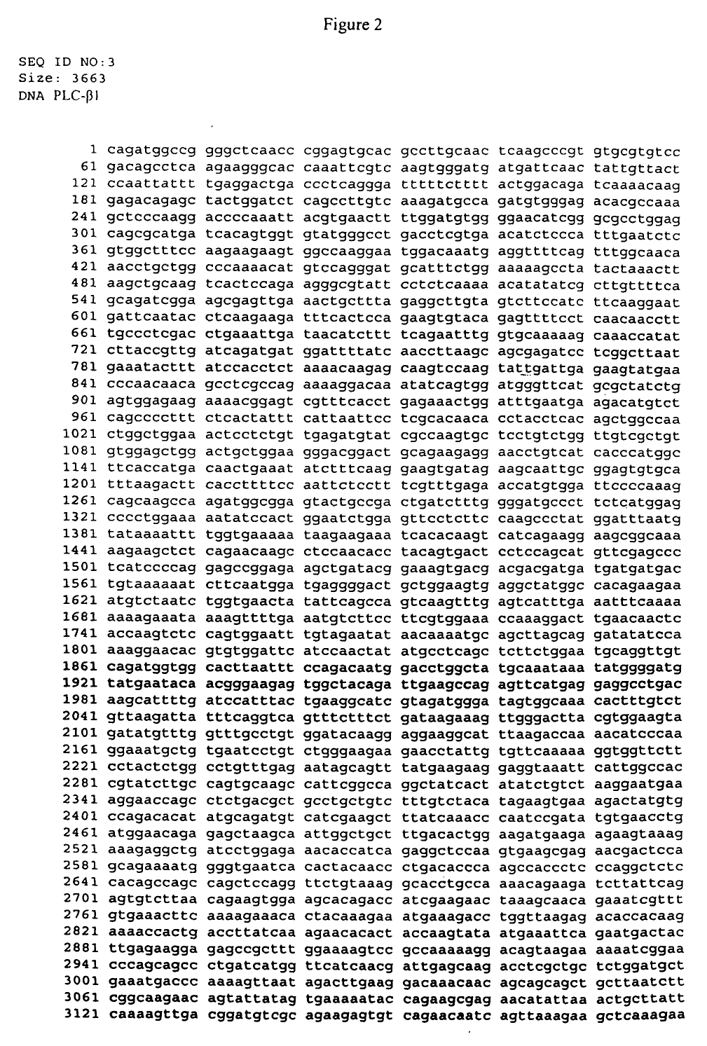

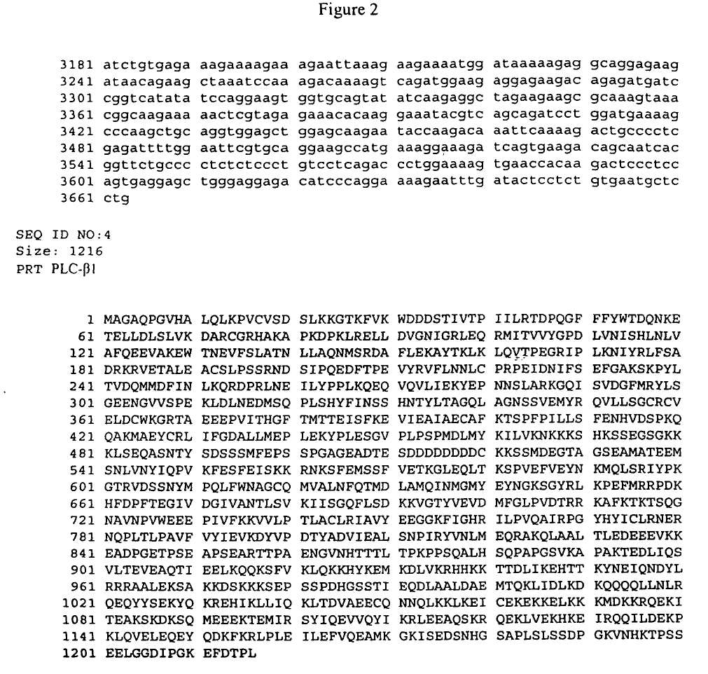

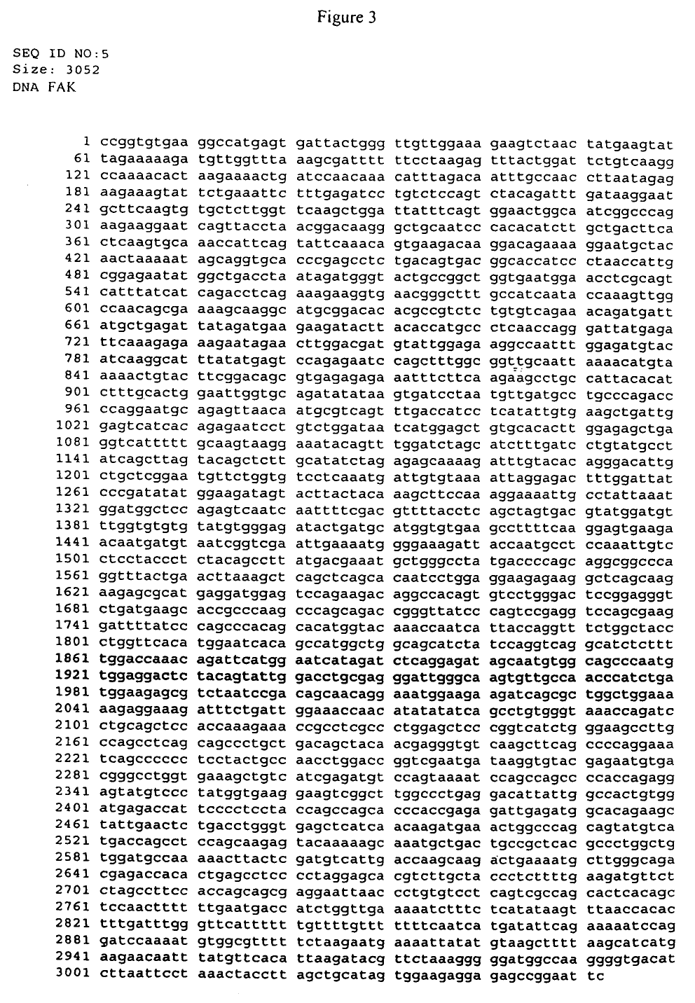

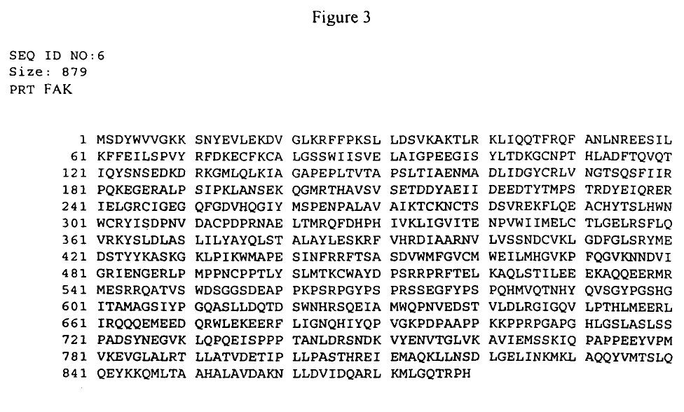

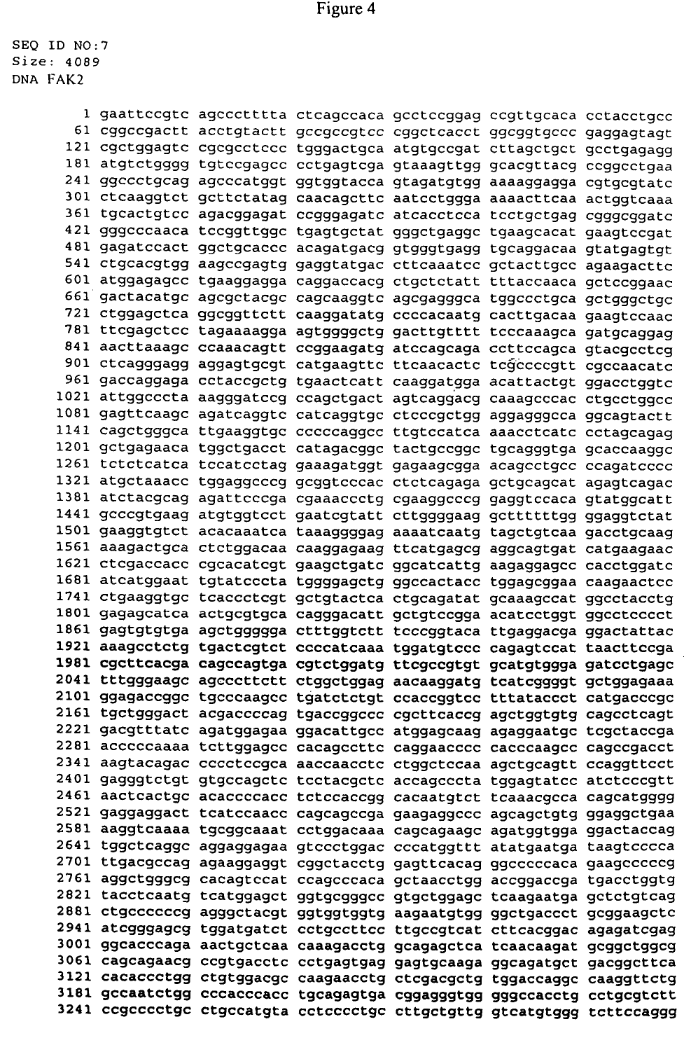

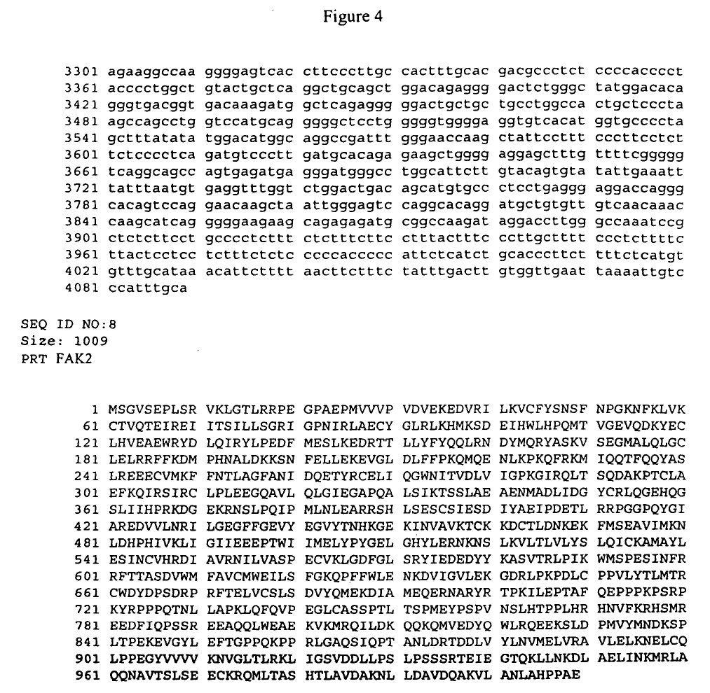

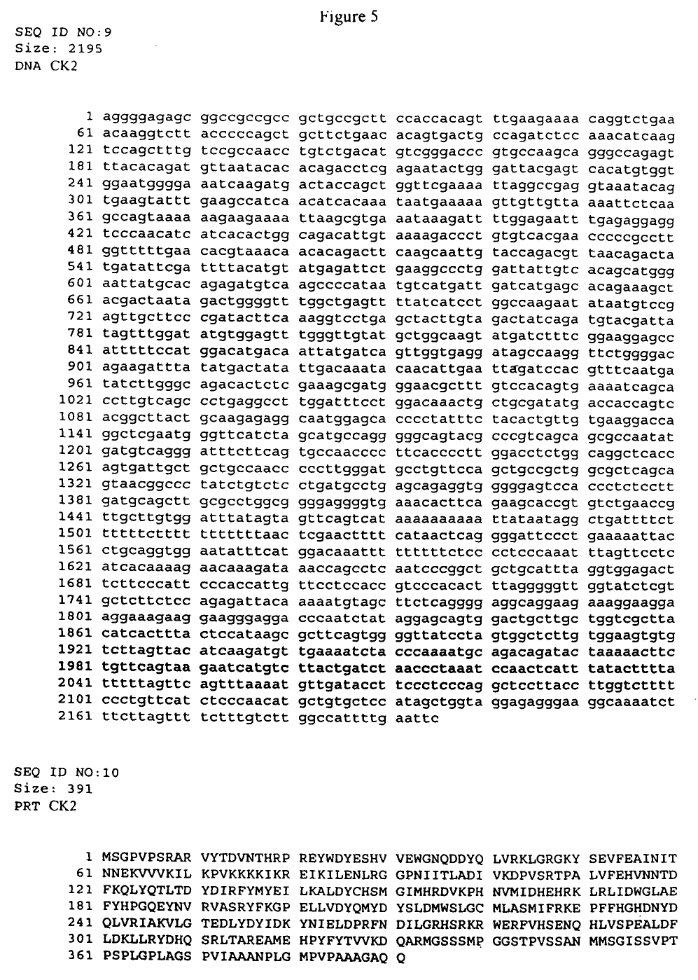

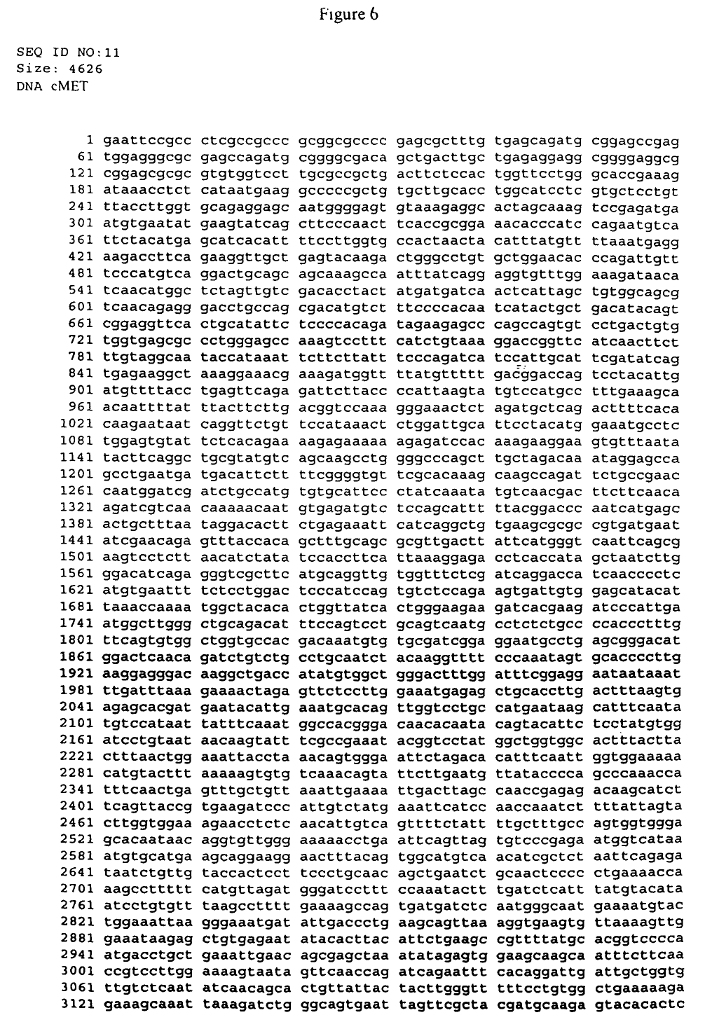

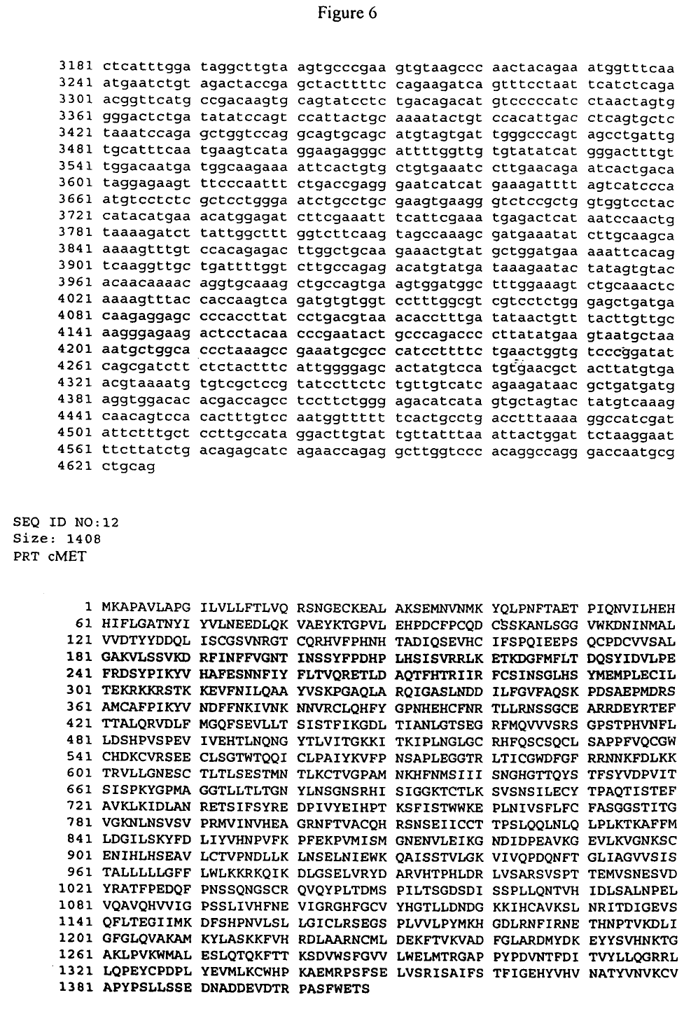

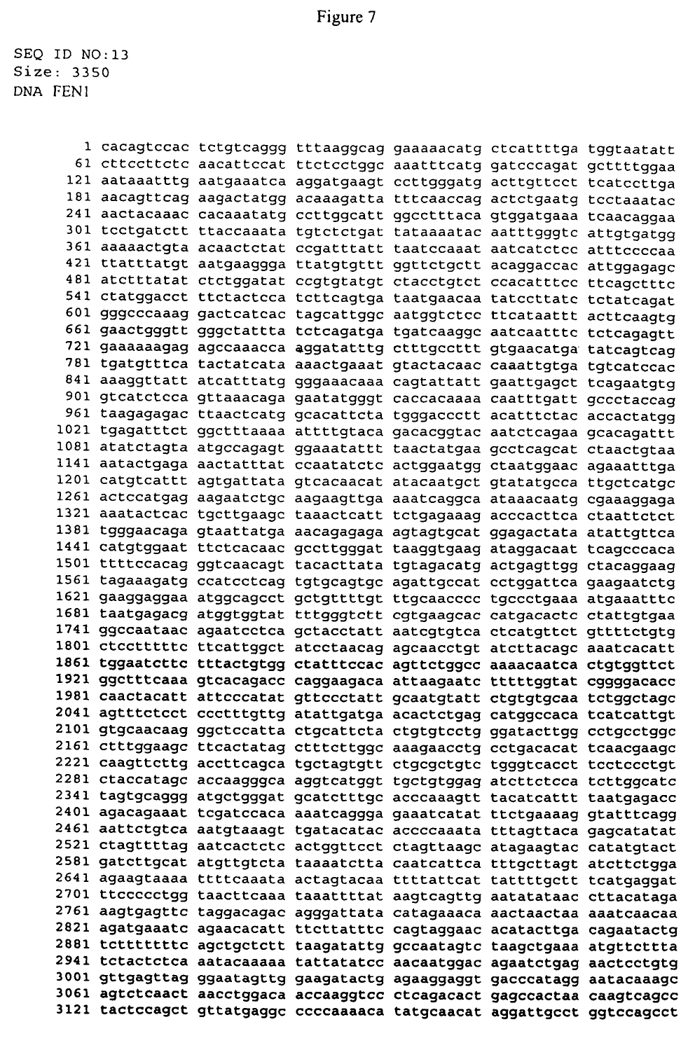

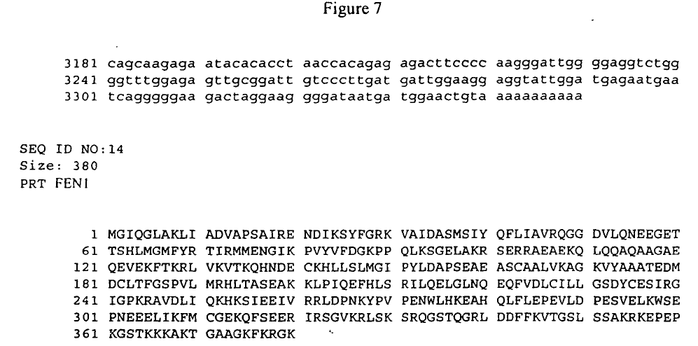

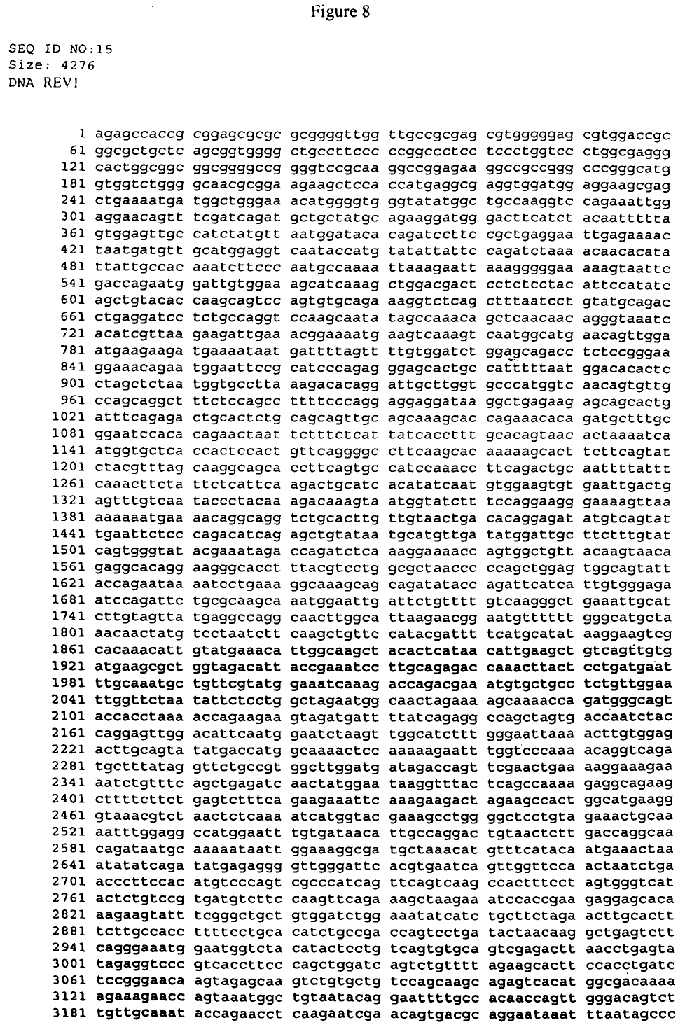

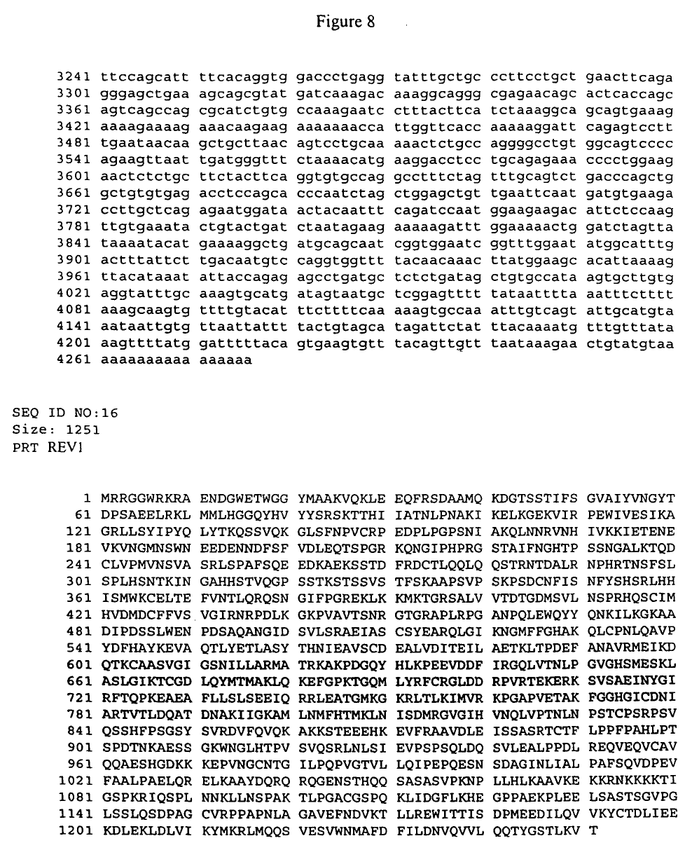

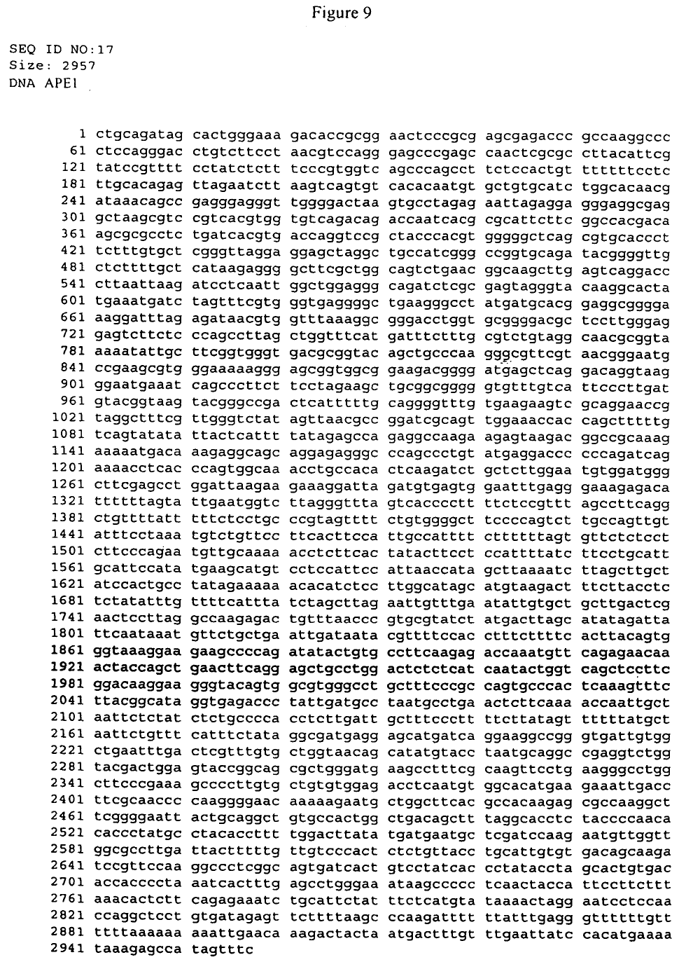

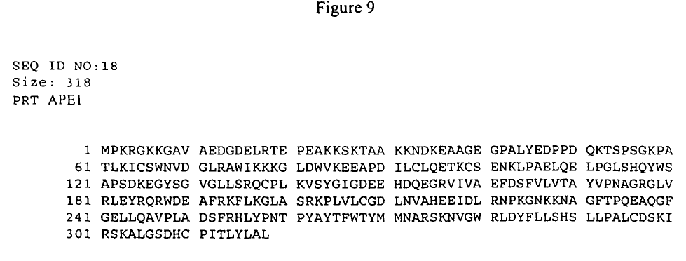

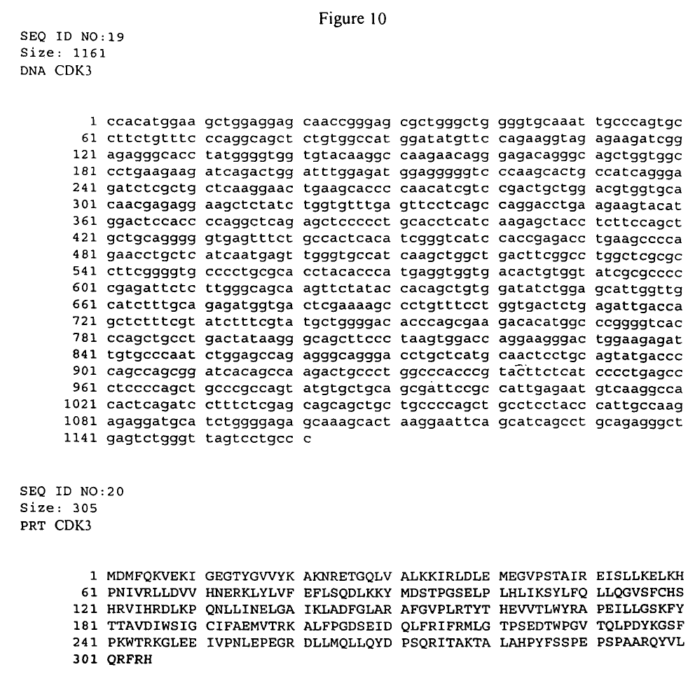

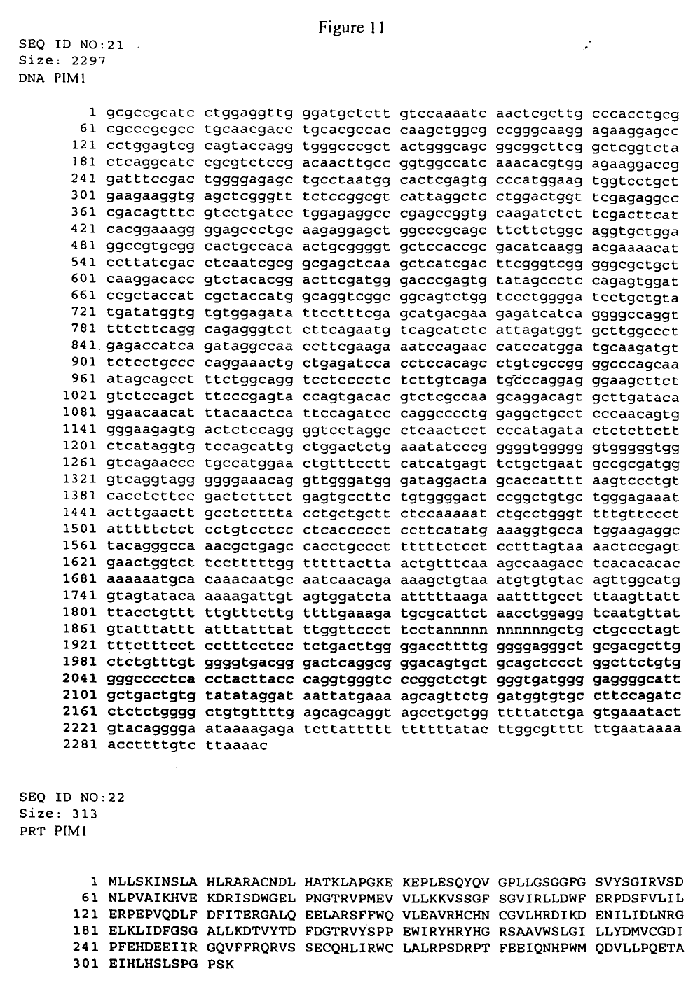

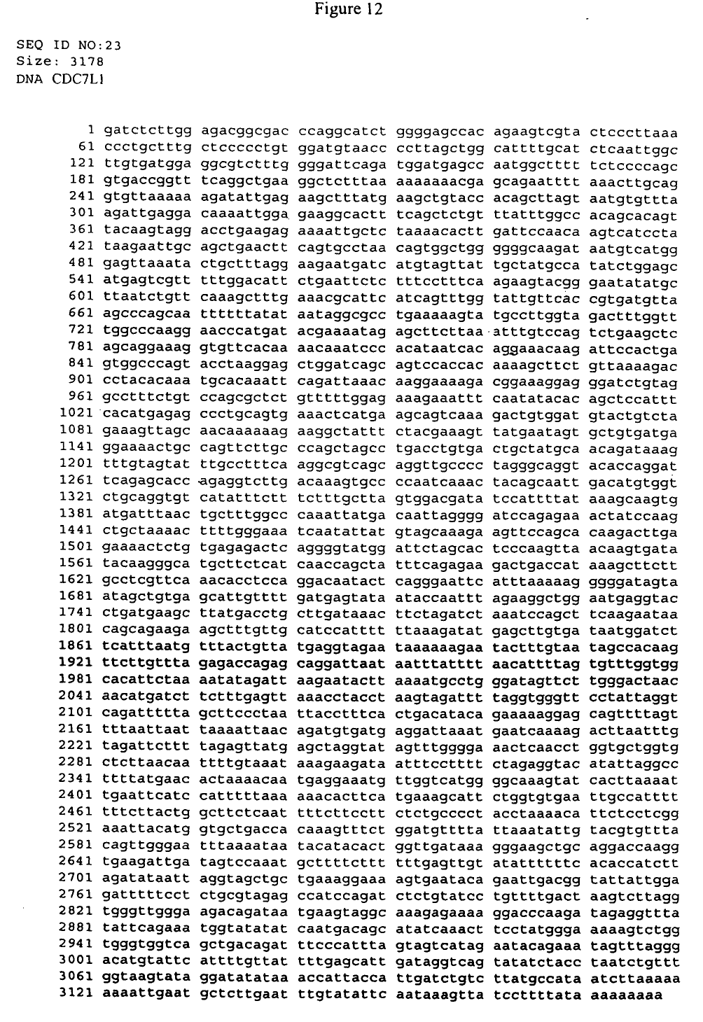

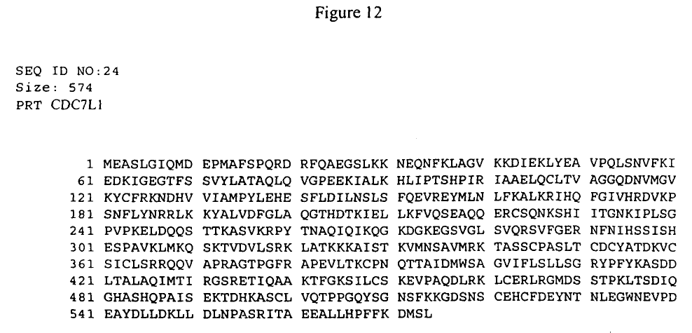

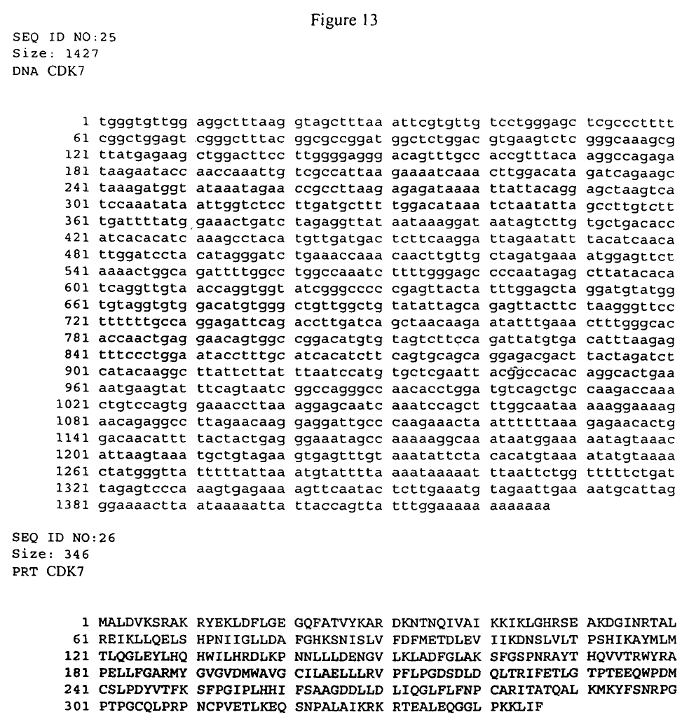

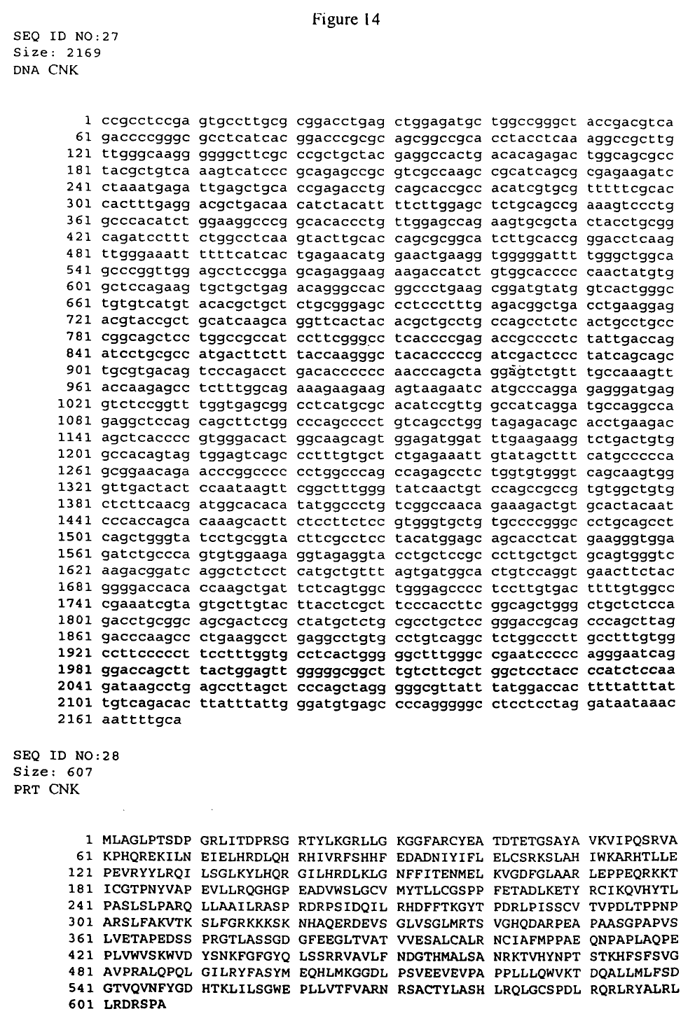

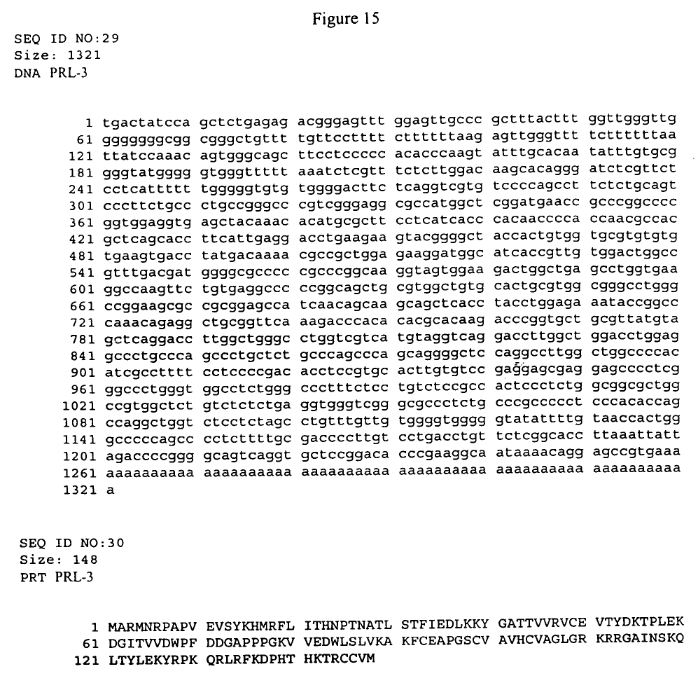

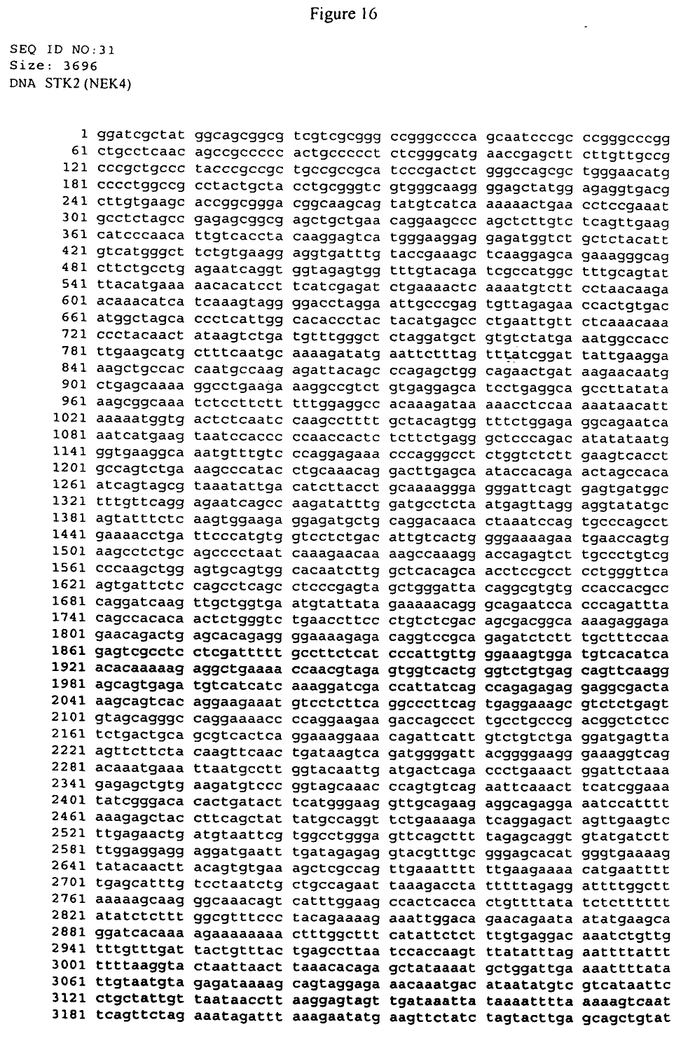

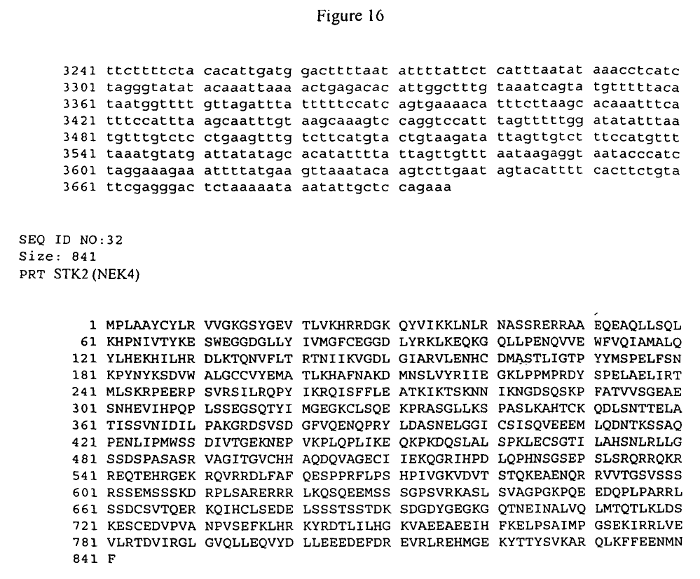

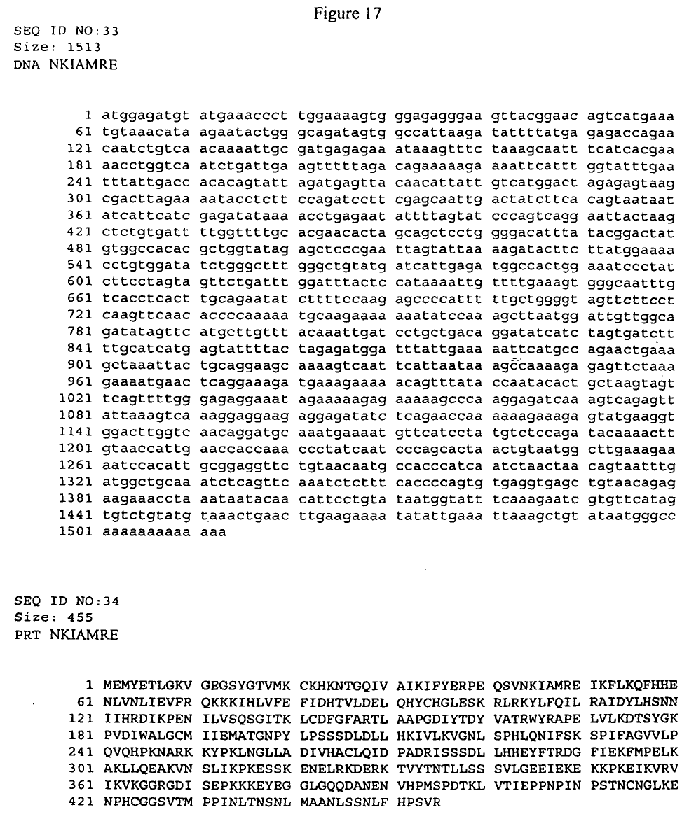

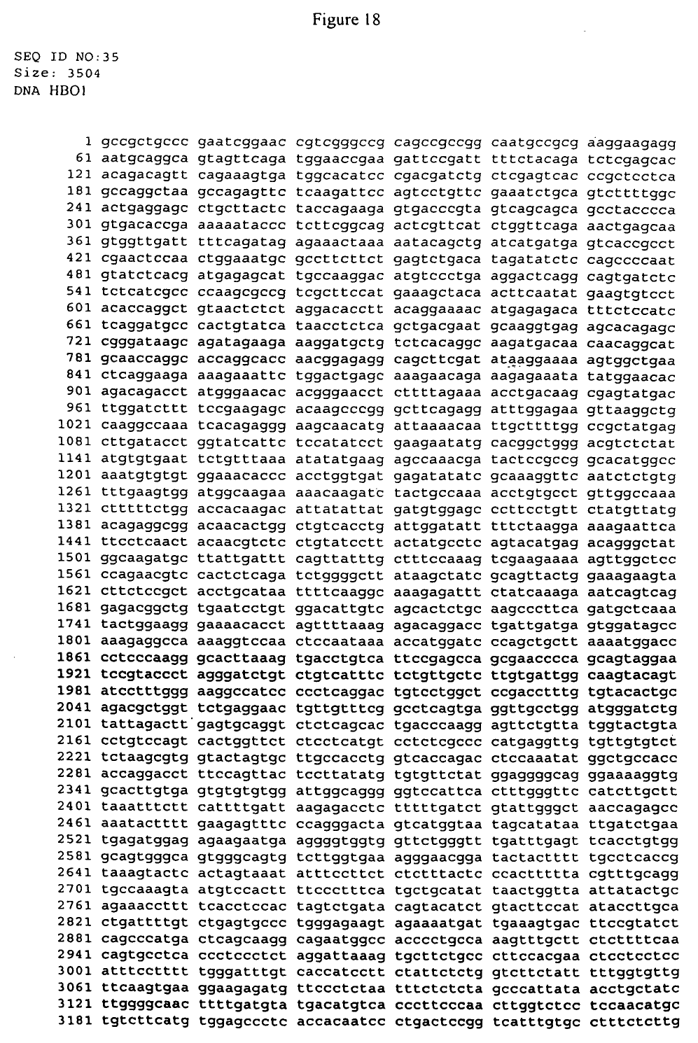

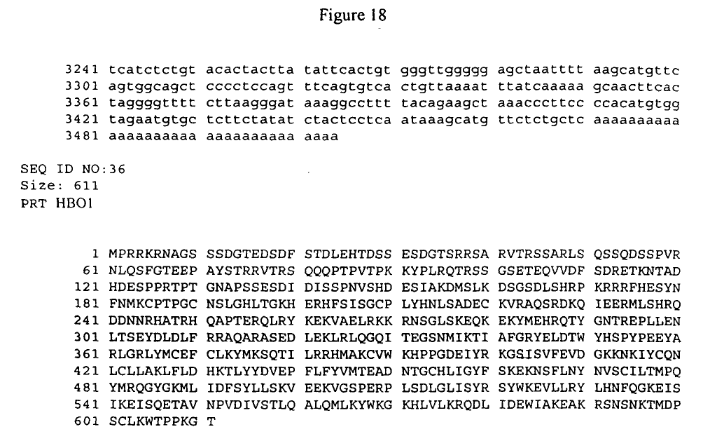

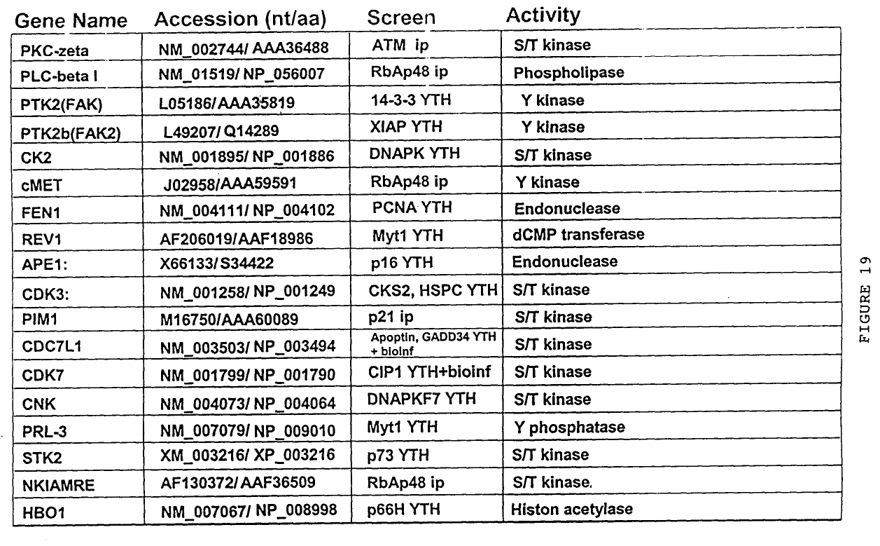

The terms “PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1” or a nucleic acid encoding “PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1” refer to nucleic acids and polypeptide polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 25, 50, 100, 200, 500, 1000, or more amino acids, to an amino acid sequence encoded by a PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 nucleic acid (for a human PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 nucleic acid sequence, see, e.g., FIGS. 1-18, SEQ ID NO:1, 3, 5, 7, 9, 11, 13, 15, 17, 19, 21, 23, 25, 27, 29, 31, 33, 35 or Accession number NM_{—}002744, NM_{—}015192, L05186, L49207, NM_{—}001895, J02958, NM_{—}004111, AF206019, X66133, NM_{—}001258, M16750, NM_{—}003503, NM_{—}001799, NM_{—}004073, NM_{—}007079, XM_{—}003216, AF130372, or NM_{—}007067 or amino acid sequence of a PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein (for a human PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein sequence, see, e.g., FIGS. 1-18, SEQ ID NO:2, 4, 6, 8, 10, 12, 14, 16, 18, 20, 22, 24, 26, 28, 30, 32, 34, 36 or Accession number AAA36488, NP_{—}056007, AAA35819, Q14289, NP_{—}001886, AAA59591, NP_{—}004102, AAF18986, S34422, NP_{—}001249, AAA60089, NP_{—}003494, NP_{—}001790, NP_{—}004064, NP_{—}009010, XP_{—}003216, AAF36509, and NP_{—}008998; (2) bind to antibodies, e.g., polyclonal antibodies, raised against an immunogen comprising an amino acid sequence of a PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein, and conservatively modified variants thereof; (3) specifically hybridize under stringent hybridization conditions to an anti-sense strand corresponding to a nucleic acid sequence encoding a PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein, and conservatively modified variants thereof; (4) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, to a PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 nucleic acid or a nucleic acid encoding the enzymatic domain. Preferably the enzymatic domain has greater than 96%, 97%, 98%, or 99% amino acid identity to the human PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 enzymatic domain of SEQ ID NO:2, 4, 6, 8, 10, 12, 14, 16, 18, 20, 22, 24, 26, 28, 30, 32, 35, or 36. A polynucleotide or polypeptide sequence is typically from a mammal including, but not limited to, primate, e.g., human; rodent, e.g., rat, mouse, hamster; cow, pig, horse, sheep, or any mammal. The nucleic acids and proteins of the invention include both naturally occurring or recombinant molecules.

The phrase “functional effects” in the context of assays for testing compounds that modulate activity of a PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein includes the determination of a parameter that is indirectly or directly under the influence of a PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1, e.g., a phenotypic or chemical effect, such as the ability to increase or decrease cellular proliferation, apoptosis, cell cycle arrest, or enzymatic activity, or e.g., a physical effect such as ligand binding or inhibition of ligand binding. A functional effect therefore includes ligand binding activity, the ability of cells to proliferate, apoptosis, and enzyme activity. “Functional effects” include in vitro, in vivo, and ex vivo activities.

By “determining the functional effect” is meant assaying for a compound that increases or decreases a parameter that is indirectly or directly under the influence of a PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein, e.g., measuring physical and chemical or phenotypic effects. Such functional effects can be measured by any means known to those skilled in the art, e.g., changes in spectroscopic characteristics (e.g., fluorescence, absorbance, refractive index); hydrodynamic (e.g., shape); chromatographic; or solubility properties for the protein; measuring inducible markers or transcriptional activation of the protein; measuring binding activity or binding assays, e.g. binding to antibodies; measuring changes in ligand or substrate binding activity; measuring cellular proliferation; measuring cell morphology, e.g., spindle formation or chromosome formation; measuring phosphorylated proteins such as histone H3 using antibodies; measuring apoptosis; measuring cell surface marker expression; measurement of changes in protein levels for PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1-associated sequences; measurement of RNA stability, identification of downstream or reporter gene expression (CAT, luciferase, β-gal, GFP and the like), e.g., via chemiluminescence, fluorescence, colorimetric reactions, antibody binding, and inducible markers.

“Inhibitors”, “activators”, and “modulators” of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 polynucleotide and polypeptide sequences are used to refer to activating, inhibitory, or modulating molecules identified using in vitro and in vivo assays of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 polynucleotide and polypeptide sequences. Inhibitors are compounds that, e.g., bind to, partially or totally block activity, decrease, prevent, delay activation, inactivate, desensitize, or down regulate the activity or expression of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 proteins, e.g., antagonists. “Activators” are compounds that increase, open, activate, facilitate, enhance activation, sensitize, agonize, or up regulate PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein activity, e.g., agonists. Inhibitors, activators, or modulators also include genetically modified versions of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 proteins, e.g., versions with altered activity, as well as naturally occurring and synthetic ligands, antagonists, agonists, antibodies, peptides, cyclic peptides, nucleic acids, siRNA molecules, antisense molecules, ribozymes, small chemical molecules and the like. Such assays for inhibitors and activators include, e.g., expressing PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein in vitro, in cells, or cell membranes, applying putative modulator compounds, and then determining the functional effects on activity, as described above.

Samples or assays comprising PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 proteins that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 is achieved when the activity value relative to the control is about 80%, preferably 50%, more preferably 25-0%. Activation of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 is achieved when the activity value relative to the control (untreated with activators) is 110%, more preferably 150%, more preferably 200-500% (i.e., two to five fold higher relative to the control), more preferably 1000-3000% higher.

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, oligonucleotide, etc., to be tested for the capacity to directly or indirectly modulation tumor cell proliferation. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

An “siRNA” refers to a nucleic acid that forms a double stranded RNA, which double stranded RNA has the ability to reduce or inhibit expression of a gene or target gene when the siRNA expressed in the same cell as the gene or target gene. “siRNA” thus refers to the double stranded RNA formed by the complementary strands. siRNA molecule and RNAi molecule are used interchangeably herein. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. In another embodiment, a “randomized siRNA” refers to a nucleic acid that forms a double stranded siRNA, wherein the sequence of the siRNA is randomized. The sequence of the siRNA can correspond to the full length target gene, or a subsequence thereof. Typically, the siRNA is at least about 15-50 nucleotides in length (e.g., each complementary sequence of the double stranded siRNA is 15-50 nucleotides in length, and the double stranded siRNA is about 15-50 base pairs in length, preferabley about 15-30 nucleotides in length, preferably about 20-30 nucleotides in length, preferably about 21-30 nucleotides in length, or about 20-25 or about 24-29 nucleotides in length, e.g., 20, 21, 22, 23, 24, 25, 26, 27, 28, 29, or 30 nucleotides in length.

“Biological sample” include sections of tissues such as biopsy and autopsy samples, and frozen sections taken for histologic purposes. Such samples include blood, sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region (e.g., nucleotide sequence SEQ ID NO:1, 3, 5, 7, 9, 11, 13, 15, 17, 19, 21, 23, 25, 27, 29, 31, 33, 35 or amino acid sequence SEQ ID NO:2, 4, 6, 8, 10, 12, 14, 16, 18, 20, 22, 24, 26, 28, 30, 32, 34, 36), when compared and aligned for maximum correspondence over a comparison window or designated region) as measured using a BLAST or BLAST 2.0 sequence comparison algorithms with default parameters described below, or by manual aligmnent and visual inspection. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher, et al., J. Biol. Chem. 273(52):35095-35101 (1998).

The terms “polypeptide,” “eptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing funictionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity, e.g., a kinase domain. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

In one embodiment, the antibody is conjugated to an “effector” moiety. The effector moiety can be any number of molecules, including labeling moieties such as radioactive labels or fluorescent labels, or can be a therapeutic moiety. In one aspect the antibody modulates the activity of the protein.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to a PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein, polymorphic variants, alleles, orthologs, and conservatively modified variants, or splice variants, or portions thereof, can be selected to obtain only those polyclonal antibodies that are specifically immunoreactive with PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 proteins and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

By “therapeutically effective dose” herein is meant a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

Assays for Proteins that Modulate Cellular Proliferation

High throughput functional genomics assays can be used to identify modulators of cellular proliferation. Such assays can monitor changes in cell surface marker expression, proliferation and differentiation, and apoptosis, using either cell lines or primary cells. Typically, the cells are contacted with a cDNA or a random peptide library (encoded by nucleic acids). In one embodiment, the peptides are cyclic or circular. The cDNA library can comprise sense, antisense, full length, and truncated cDNAs. The peptide library is encoded by nucleic acids. The effect of the cDNA or peptide library on the phenotype of cellular proliferation is then monitored, using an assay as described above. The effect of the cDNA or peptide can be validated and distinguished from somatic mutations, using, e.g., regulatable expression of the nucleic acid such as expression from a tetracycline promoter. cDNAs and nucleic acids encoding peptides can be rescued using techniques known to those of skill in the art, e.g., using a sequence tag.

Proteins interacting with the peptide or with the protein encoded by the cDNA (e.g., PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1) can be isolated using a yeast two-hybrid system, mammalian two hybrid system, immunoprecipitation or affinity chromatography of complexed proteins followed by mass spectrometry, or phage display screen, etc. Targets so identified can be further used as bait in these assays to identify additional members of the cellular proliferation pathway, which members are also targets for drug development (see, e.g., Fields et al., Nature 340:245 (1989); Vasavada et al., Proc. Nat 'l Acad. Sci. USA 88:10686 (1991); Fearon et al., Proc. Nat'l Acad. Sci. USA 89:7958 (1992); Dang et al., Mol. Cell. Biol. 11:954 (1991); Chien et al., Proc. Nat 'l Acad. Sci. USA 9578 (1991); and U.S. Pat. Nos. 5,283,173, 5,667,973, 5,468,614, 5,525,490, and 5,637,463).

Suitable cell lines include A549, HeLa, Colo205, H1299, MCF7, MDA-MB-231, PC3, HMEC, PrEC. Cell surface markers can be assayed using fluorescently labeled antibodies and FACS. Cell proliferation can be measured using ³H-thymidine incorporation, cell count by dye inclusion, MTT assay, BrdU incorporation, Cell Tracker assay. Apoptosis can be measured using dye inclusion, or by assaying for DNA laddering, increases in intracellular calcium, or caspase activation. Growth factor production can be measured using an immunoassay such as ELISA.

cDNA libraries are made from any suitable source. Libraries encoding random peptides are made according to techniques well known to those of skill in the art (see, e.g., U.S. Pat. Nos. 6,153,380, 6,114,111, and 6,180,343). Any suitable vector can be used for the cDNA and peptide libraries, including, e.g., retroviral vectors.

Isolation of Nucleic Acids Encoding PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 Family Members

This invention relies on routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 nucleic acids, polymorphic variants, orthologs, and alleles that are substantially identical to an amino acid sequence encoded by SEQ ID NO:2, 4, 6, 8, 10, 12, 14, 16, 18, 20, 22, 24, 26, 28, 30, 32, 34, or 36 can be isolated using PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 nucleic acid probes and oligonucleotides under stringent hybridization conditions, by screening libraries. Alternatively, expression libraries can be used to clone PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein, polymorphic variants, orthologs, and alleles by detecting expressed homologs immunologically with antisera or purified antibodies made against human PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 or portions thereof.

To make a cDNA library, one should choose a source that is rich in PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 RNA. The mRNA is then made into cDNA using reverse transcriptase, ligated into a recombinant vector, and transfected into a recombinant host for propagation, screening and cloning. Methods for making and screening cDNA libraries are well known (see, e.g., Gubler & Hoffman, Gene 25:263-269 (1983); Sambrook et al., supra; Ausubel et al., supra).

For a genomic library, the DNA is extracted from the tissue and either mechanically sheared or enzymatically digested to yield fragments of about 12-20 kb. The fragments are then separated by gradient centrifugation from undesired sizes and are constructed in bacteriophage lambda vectors. These vectors and phage are packaged in vitro. Recombinant phage are analyzed by plaque hybridization as described in Benton & Davis, Science 196:180-182 (1977). Colony hybridization is carried out as generally described in Grunstein et al., Proc. Natl. Acad. Sci. USA., 72:3961-3965 (1975).

An alternative method of isolating PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 nucleic acid and its orthologs, alleles, mutants, polymorphic variants, and conservatively modified variants combines the use of synthetic oligonucleotide primers and amplification of an RNA or DNA template (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify nucleic acid sequences of human PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 directly from mRNA, from cDNA, from genomic libraries or cDNA libraries. Degenerate oligonucleotides can be designed to amplify PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 homologs using the sequences provided herein. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 encoding mRNA in physiological samples, for nucleic acid sequencing, or for other purposes. Genes amplified by the PCR reaction can be purified from agarose gels and cloned into an appropriate vector.

Gene expression of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 can also be analyzed by techniques known in the art, e.g., reverse transcription and amplification of mRNA, isolation of total RNA or poly A⁺ RNA, northern blotting, dot blotting, in situ hybridization, RNase protection, high density polynucleotide array technology, e.g., and the like.

Nucleic acids encoding PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein can be used with high density oligonucleotide array technology (e.g., GeneChip™) to identify PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein, orthologs, alleles, conservatively modified variants, and polymorphic variants in this invention. In the case where the homologs being identified are linked to modulation of cellular proliferation, they can be used with GeneChip™ as a diagnostic tool in detecting the disease in a biological sample, see, e.g., Gunthand et al., AIDS Res. Hum. Retroviruses 14: 869-876 (1998); Kozal et al., Nat. Med. 2:753-759 (1996); Matson et al., Anal. Biochem. 224:110-106 (1995); Lockhart et al., Nat. Biotechnol. 14:1675-1680 (1996); Gingeras et al., Genome Res. 8:435-448 (1998); Hacia et al., Nucleic Acids Res. 26:3865-3866 (1998).

The gene for PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 is typically cloned into intermediate vectors before transformation into prokaryotic or eukaryotic cells for replication and/or expression. These intermediate vectors are typically prokaryote vectors, e.g., plasmids, or shuttle vectors.

Expression in Prokaryotes and Eukaryotes

To obtain high level expression of a cloned gene, such as those cDNAs encoding PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1, one typically subclones PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al., and Ausubel et al, supra. Bacterial expression systems for expressing the PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available. In one preferred embodiment, retroviral expression systems are used in the present invention.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 encoding nucleic acid in host cells. A typical expression cassette thus contains a promoter operably linked to the nucleic acid sequence encoding PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 and signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc. Sequence tags may be included in an expression cassette for nucleic acid rescue. Markers such as fluorescent proteins, green or red fluorescent protein, β-gal, CAT, and the like can be included in the vectors as markers for vector transduction.

Expression vectors containing regulatory elements from eukaryotic viruses are typically used in eukaryotic expression vectors, e.g., SV40 vectors, papilloma virus vectors, retroviral vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Expression of proteins from eukaryotic vectors can be also be regulated using inducible promoters. With inducible promoters, expression levels are tied to the concentration of inducing agents, such as tetracycline or ecdysone, by the incorporation of response elements for these agents into the promoter. Generally, high level expression is obtained from inducible promoters only in the presence of the inducing agent; basal expression levels are minimal.

In one embodiment, the vectors of the invention have a regulatable promoter, e.g., tet-regulated systems and the RU-486 system (see, e.g., Gossen & Bujard, Proc. Nat'l Acad. Sci. USA 89:5547 (1992); Oligino et al., Gene Ther. 5:491-496 (1998); Wang et al., Gene Ther. 4:432-441 (1997); Neering et al., Blood 88:1147-1155 (1996); and Rendahl et al., Nat. Biotechnol. 16:757-761 (1998)). These impart small molecule control on the expression of the candidate target nucleic acids. This beneficial feature can be used to determine that a desired phenotype is caused by a transfected cDNA rather than a somatic mutation.

Some expression systems have markers that provide gene amplification such as thymidine kinase and dihydrofolate reductase. Alternatively, high yield expression systems not involving gene amplification are also suitable, such as using a baculovirus vector in insect cells, with a PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 encoding sequence under the direction of the polyhedrin promoter or other strong baculovirus promoters.

The elements that are typically included in expression vectors also include a replicon that functions in E. coli , a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g. Morrison, J. Bact. 132:349-351 (1977); Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1.

After the expression vector is introduced into the cells, the transfected cells are cultured under conditions favoring expression of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1, which is recovered from the culture using standard techniques identified below.

Purification of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 Polypeptides

Either naturally occurring or recombinant PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 can be purified for use in functional assays. Naturally occurring PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 can be purified, e.g., from human tissue. Recombinant PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 can be purified from any suitable expression system.

The PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al.; supra).

A number of procedures can be employed when recombinant PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein is being purified. For example, proteins having established molecular adhesion properties can be reversible fused to the PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein. With the appropriate ligand or substrate, e.g., antiphospho S/T antibodies or anti-PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 antibodies, PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein can be selectively adsorbed to a purification column and then freed from the column in a relatively pure form. The fused protein is then removed by enzymatic activity. Finally, PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein could be purified using immunoaffinity columns. Recombinant PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein can be purified from any suitable source, include yeast, insect, bacterial, and mammalian cells.

A. Purification of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 from Recombinant Bacteria

Recombinant proteins are expressed by transformed bacteria in large amounts, typically after promoter induction; but expression can be constitutive. Promoter induction with IPTG is one example of an inducible promoter system. Bacteria are grown according to standard procedures in the art. Fresh or frozen bacteria cells are used for isolation of protein.

Proteins expressed in bacteria may form insoluble aggregates (“inclusion bodies”). Several protocols are suitable for purification of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein inclusion bodies. For example, purification of inclusion bodies typically involves the extraction, separation and/or purification of inclusion bodies by disruption of bacterial cells, e.g., by incubation in a buffer of 50 mM TRIS/HCL pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies may be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of immunologically and/or biologically active protein. Other suitable buffers are known to those skilled in the art. Human PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 proteins are separated from other bacterial proteins by standard separation techniques, e.g., with Ni—NTA agarose resin.

Alternatively, it is possible to purify PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein from bacteria periplasm. After lysis of the bacteria, when the PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein exported into the periplasm of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

B. Standard Protein Separation Techniques for Purifying PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 proteins

Solubility Fractionation

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

Size Differential Filtration

The molecular weight of the PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 proteins can be used to isolate it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

Column Chromatography

The PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 proteins can also be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

Assays for Modulators of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 Protein

A. Assays

Modulation of a PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein, and corresponding modulation of cellular, e.g., tumor cell, proliferation, can be assessed using a variety of in vitro and in vivo assays, including cell-based models. Such assays can be used to test for inhibitors and activators of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein, and, consequently, inhibitors and activators of cellular proliferation, including modulators of chemotherapeutic sensitivity and toxicity. Such modulators of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein are useful for treating disorders related to pathological cell proliferation, e.g., cancer. Modulators of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein are tested using either recombinant or naturally occurring PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1, preferably human PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1.

Preferably, the PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein will have the sequence as encoded by SEQ ID NO:2, 4, 6, 8, 10, 12, 14, 16, 18, 20, 22, 24, 26, 28, 30, 32, 34, 36 or a conservatively modified variant thereof. Alternatively, the PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein of the assay will be derived from a eukaryote and include an amino acid subsequence having substantial amino acid sequence identity to SEQ ID NO:2, 4, 6, 8, 10, 12, 14, 16, 18, 20, 22, 24, 26, 28, 30, 32, 34, 36. Generally, the amino acid sequence identity will be at least 60%, preferably at least 65%, 70%, 75%, 80%, 85%, or 90%, most preferably at least 95%.

Measurement of cellular proliferation modulation with PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein or a cell expressing PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein, either recombinant or naturally occurring, can be performed using a variety of assays, in vitro, in vivo, and ex vivo, as described herein. A suitable physical, chemical or phenotypic change that affects activity, e.g., enzymatic activity such as kinase activity, cell proliferation, or ligand binding can be used to assess the influence of a test compound on the polypeptide of this invention. When the functional effects are determined using intact cells or animals, one can also measure a variety of effects, such as, ligand binding, kinase activity, transcriptional changes to both known and uncharacterized genetic markers (e.g., northern blots), changes in cell metabolism, changes related to cellular proliferation, cell surface marker expression, DNA synthesis, marker and dye dilution assays (e.g., GFP and cell tracker assays), contact inhibition, tumor growth in nude mice, etc.

In Vitro Assays

Assays to identify compounds with PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 modulating activity can be performed in vitro. Such assays can use full length PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein or a variant thereof (see, e.g., SEQ ID NO:2, 4, 6, 8, 10, 12, 14, 16, 18, 20, 22, 24, 26, 28, 30, 32, 34, 36), or a mutant thereof, or a fragment of a PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein, such as a kinase domain. Purified recombinant or naturally occurring PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein can be used in the in vitro methods of the invention. In addition to purified PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein, the recombinant or naturally occurring PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein can be part of a cellular lysate or a cell membrane. As described below, the binding assay can be either solid state or soluble. Preferably, the protein or membrane is bound to a solid support, either covalently or non-covalently. Often, the in vitro assays of the invention are substrate or ligand binding or affinity assays, either non-competitive or competitive. Other in vitro assays include measuring changes in spectroscopic (e.g., fluorescence, absorbance, refractive index), hydrodynamic (e.g., shape), chromatographic, or solubility properties for the protein. Other in vitro assays include enzymatic activity assays, such as phosphorylation or autophosphorylation assays.

In one embodiment, a high throughput binding assay is performed in which the PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein or a fragment thereof is contacted with a potential modulator and incubated for a suitable amount of time. In one embodiment, the potential modulator is bound to a solid support, and the PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein is added. In another embodiment, the PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein is bound to a solid support. A wide variety of modulators can be used, as described below, including small organic molecules, peptides, antibodies, and PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 ligand analogs. A wide variety of assays can be used to identify PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1-modulator binding, including labeled protein-protein binding assays, electrophoretic mobility shifts, immunoassays, enzymatic assays such as kinase assays, and the like. In some cases, the binding of the candidate modulator is determined through the use of competitive binding assays, where interference with binding of a known ligand or substrate is measured in the presence of a potential modulator. Either the modulator or the known ligand or substrate is bound first, and then the competitor is added. After the PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein is washed, interference with binding, either of the potential modulator or of the known ligand or substrate, is determined. Often, either the potential modulator or the known ligand or substrate is labeled.

Cell-Based In Vivo Assays

In another embodiment, PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein is expressed in a cell, and functional, e.g., physical and chemical or phenotypic, changes are assayed to identify PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 and modulators of cellular proliferation, e.g., tumor cell proliferation. Cells expressing PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 proteins can also be used in binding assays and enzymatic assays. Any suitable functional effect can be measured, as described herein. For example, cellular morphology (e.g., cell volume, nuclear volume, cell perimeter, and nuclear perimeter), ligand binding, kinase activity, apoptosis, cell surface marker expression, cellular proliferation, GFP positivity and dye dilution assays (e.g., cell tracker assays with dyes that bind to cell membranes), DNA synthesis assays (e.g., ³H-thymidine and fluorescent DNA-binding dyes such as BrdU or Hoescht dye with FACS analysis), are all suitable assays to identify potential modulators using a cell based system. Suitable cells for such cell based assays include both primary cancer or tumor cells and cell lines, as described herein, e.g., A549 (lung), MCF7 (breast, p53 wild-type), H1299 (lung, p53 null), Hela (cervical), PC3 (prostate, p53 mutant), MDA-MB-231 (breast, p53 wild-type). Cancer cell lines can be p53 mutant, p53 null, or express wild type p53. The PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein can be naturally occurring or recombinant. Also, fragments of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 orchimeric PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 proteins with enzymatic activity can be used in cell based assays.

Cellular PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 polypeptide levels can be determined by measuring the level of protein or mRNA. The level of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein or proteins related to PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 are measured using immunoassays such as western blotting, ELISA and the like with an antibody that selectively binds to the PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 polypeptide or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 expression can be measured using a reporter gene system. Such a system can be devised using a PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein promoter operably linked to a reporter gene such as chloramphenicol acetyltransferase, firefly luciferase, bacterial luciferase, β-galactosidase and alkaline phosphatase. Furthermore, the protein of interest can be used as an indirect reporter via attachment to a second reporter such as red or green fluorescent protein (see, e.g., Mistili & Spector, Nature Biotechnology 15:961-964 (1997)). The reporter construct is typically transfected into a cell. After treatment with a potential modulator, the amount of reporter gene transcription, translation, or activity is measured according to standard techniques known to those of skill in the art.

Animal Models

Animal models of cellular proliferation also find use in screening for modulators of cellular proliferation. Similarly, transgenic animal technology including gene knockout technology, for example as a result of homologous recombination with an appropriate gene targeting vector, or gene overexpression, will result in the absence or increased expression of the PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1. The same technology can also be applied to make knock-out cells. When desired, tissue-specific expression or knockout of the PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein may be necessary. Transgenic animals generated by such methods find use as animal models of cellular proliferation and are additionally useful in screening for modulators of cellular proliferation.

Knock-out cells and transgenic mice can be made by insertion of a marker gene or other heterologous gene into an endogenous PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 gene site in the mouse genome via homologous recombination. Such mice can also be made by substituting an endogenous PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 with a mutated version of the PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 gene, or by mutating an endogenous PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1, e.g., by exposure to carcinogens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987).

Exemplary Assays

Enzymatic Activity Assays—In Vitro or Cell Based

In one embodiment, enzymatic assays using PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 can be used to identify modulators of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 activity, or to identify proteins that bind to PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1, e.g., PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 substrates. Full length wild type PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1, mutant PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1, or the PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 enzymatic domain can be used in these assays. Such assays can be performed in vitro, using recombinant PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 or cellular lysates comprising endogenous or recombinant PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1, or can be cell-based.

Soft Agar Growth or Colony Formation in Suspension

Normal cells require a solid substrate to attach and grow. When the cells are transformed, they lose this phenotype and grow detached from the substrate. For example, transformed cells can grow in stirred suspension culture or suspended in semi-solid media, such as semi-solid or soft agar. The transformed cells, when transfected with tumor suppressor genes, regenerate normal phenotype and require a solid substrate to attach and grow.

Soft agar growth or colony formation in suspension assays can be used to identify PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 modulators. Typically, transformed host cells (e.g., cells that grow on soft agar) are used in this assay. For example, RKO or HCT116 cell lines can be used. Techniques for soft agar growth or colony formation in suspension assays are described in Freshney, Culture of Animal Cells a Manual of Basic Technique, 3^{rd} ed., Wiley-Liss, New York (1994), herein incorporated by reference. See also, the methods section of Garkavtsev et al. (1996), supra, herein incorporated by reference.

Contact Inhibition and Density Limitation of Growth

Normal cells typically grow in a flat and organized pattern in a petri dish until they touch other cells. When the cells touch one another, they are contact inhibited and stop growing. When cells are transformed, however, the cells are not contact inhibited and continue to grow to high densities in disorganized foci. Thus, the transformed cells grow to a higher saturation density than normal cells. This can be detected morphologically by the formation of a disoriented monolayer of cells or rounded cells in foci within the regular pattern of normal surrounding cells. Alternatively, labeling index with [³H]-thymidine at saturation density can be used to measure density limitation of growth. See Freshney (1994), supra. The transformed cells, when contacted with cellular proliferation modulators, regenerate a normal phenotype and become contact inhibited and would grow to a lower density.

Contact inhibition and density limitation of growth assays can be used to identify PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 modulators which are capable of inhibiting abnormal proliferation and transformation in host cells. Typically, transformed host cells (e.g., cells that are not contact inhibited) are used in this assay. For example, RKO or HCT116 cell lines can be used. In this assay, labeling index with [³H]-thymidine at saturation density is a preferred method of measuring density limitation of growth. Transformed host cells are contacted with a potential PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 modulator and are grown for 24 hours at saturation density in non-limiting medium conditions. The percentage of cells labeling with [³H]-thymidine is determined autoradiographically. See, Freshney (1994), supra. The host cells contacted with a PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 modulator would give arise to a lower labeling index compared to control (e.g., transformed host cells transfected with a vector lacking an insert).

Growth Factor or Serum Dependence

Growth factor or serum dependence can be used as an assay to identify PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 modulators. Transformed cells have a lower serum dependence than their normal counterparts (see, e.g., Temin, J. Natl. Cancer Insti. 37:167-175 (1966); Eagle et al., J. Exp. Med. 131:836-879 (1970)); Freshney, supra. This is in part due to release of various growth factors by the transformed cells. When transformed cells are contacted with a PKC-ζ, PLC-β1, FAK, FAK2, CK2, cMET, FEN1, REV1, APE1, CDK3, PIM1, CDC7L1, CDK7, CNK, PRL3, STK2 (NEK4), NKIAMRE, or HBO1 modulator, the cells would reacquire serum dependence and would release growth factors at a lower level.

Tumor Specific Markers Levels

Tumor cells release an increased amount of certain factors (hereinafter “tumor specific markers”) than their normal counterparts. For example, plasminogen activator (PA) is released from human glioma at a higher level than from normal brain cells (see, e.g., Gullino, Angiogenesis, tumor vascularization, and potential interference with tumor growth. In Mihich (ed.): “Biological Responses in Cancer.” New York, Academic Press, pp. 178-184 (1985)). Similarly, tumor angiogenesis factor (TAF) is released at a higher level in tumor cells than their normal counterparts. See, e.g., Folkman, Angiogenesis and cancer, Sem Cancer Biol. (1992)).

Tumor specific markers can be assayed to identify PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 modulators which decrease the level of release of these markers from host cells. Typically, transformed or tumorigenic host cells are used. Various techniques which measure the release of these factors are described in Freshney (1994), supra. Also, see, Unkless et al., J. Biol. Chem. 249:4295-4305 (1974); Strickland & Beers, J. Biol. Chem. 251:5694-5702 (1976); Whur et al., Br. J. Cancer 42:305-312 (1980); Gulino, Angiogenesis, tumor vascularization, and potential interference with tumor growth. In Mihich, E. (ed): “Biological Responses in Cancer.” New York, Plenum (1985); Freshney Anticancer Res. 5:111-130 (1985).

Invasiveness into Matrigel

The degree of invasiveness into Matrigel or some other extracellular matrix constituent can be used as an assay to identify PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 modulators which are capable of inhibiting abnormal cell proliferation and tumor growth. Tumor cells exhibit a good correlation between malignancy and invasiveness of cells into Matrigel or some other extracellular matrix constituent. In this assay, tumorigenic cells are typically used as host cells. Therefore, PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 modulators can be identified by measuring changes in the level of invasiveness between the host cells before and after the introduction of potential modulators. If a compound modulates PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1, its expression in tumorigenic host cells would affect invasiveness.

Techniques described in Freshney (1994), supra, can be used. Briefly, the level of invasion of host cells can be measured by using filters coated with Matrigel or some other extracellular matrix constituent. Penetration into the gel, or through to the distal side of the filter, is rated as invasiveness, and rated histologically by number of cells and distance moved, or by prelabeling the cells with ¹²⁵I and counting the radioactivity on the distal side of the filter or bottom of the dish. See, e.g., Freshney (1984), supra.

Apoptosis Analysis

Apoptosis analysis can be used as an assay to identify PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 modulators. In this assay, cell lines, such as RKO or HCT116, can be used to screen PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 modulators. Cells are contacted with a putative PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 modulator. The cells can be co-transfected with a construct comprising a marker gene, such as a gene that encodes green fluorescent protein, or a cell tracker dye. The apoptotic change can be determined using methods known in the art, such as DAPI staining and TUNEL assay using a fluorescent microscope. For TUNEL assay, commercially available kit can be used (e.g., Fluorescein FragEL DNA Fragmentation Detection Kit (Oncogene Research Products, Cat.# QIA39)+Tetramethyl-rhodamine-5-dUTP (Roche, Cat. # 1534 378)). Cells contacted with PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 modulators would exhibit, e.g., an increased apoptosis compared to control.

Cell Cycle Arrest Analysis

Cell cycle arrest can be used as an assay to identify PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 modulators. In this assay, cell lines, such as RKO or HCT116, can be used to screen PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 modulators. The cells can be co-transfected with a construct comprising a marker gene, such as a gene that encodes green fluorescent protein, or a cell tracker dye. Methods known in the art can be used to measure the degree of cell cycle arrest. For example, a propidium iodide signal can be used as a measure for DNA content to determine cell cycle profiles on a flow cytometer. The percent of the cells in each cell cycle can be calculated. Cells contacted with a PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 modulator would exhibit, e.g., a higher number of cells that are arrested in G₁/G₀ phase, G₁/S phase, S/G₂ phase, G₂/M phase, or M/G₂ phase compared to control.

Tumor Growth In Vivo

Effects of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 modulators on cell growth can be tested in transgenic or immune-suppressed mice (e.g., xenograft models). Knock-out transgenic mice can be made, in which the endogenous PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 gene is disrupted. Such knock-out mice can be used to study effects of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1, e.g., as a cancer model, as a means of assaying in vivo for compounds that modulate PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1, and to test the effects of restoring a wild-type or mutant PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 to a knock-out mice.

Knock-out cells and transgenic mice can be made by insertion of a marker gene or other heterologous gene into the endogenous PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 gene site in the mouse genome via homologous recombination. Such mice can also be made by substituting the endogenous PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 with a mutated version of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1, or by mutating the endogenous PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1, e.g., by exposure to carcinogens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987). These knock-out mice can be used as hosts to test the effects of various PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 modulators on cell growth.

Alternatively, various immune-suppressed or immune-deficient host animals can be used. For example, genetically athymic “nude” mouse (see, e.g., Giovanella et al., J. Natl. Cancer Inst. 52:921 (1974)), a SCID mouse, a thymectomized mouse, or an irradiated mouse (see, e.g., Bradley et al., Br. J. Cancer 38:263 (1978); Selby et al, Br. J. Cancer 41:52 (1980)) can be used as a host for, e.g., xenografts. Transplantable tumor cells (typically about 10⁶ cells), such as, for example, human tumor cells, injected into isogenic hosts will produce invasive tumors in a high proportions of cases, while normal cells of similar origin will not. Hosts are treated with PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 modulators, e.g., by injection. After a suitable length of time, preferably 4-8 weeks, tumor growth is measured (e.g., by volume or by its two largest dimensions) and compared to the control. Tumors that have statistically significant reduction (using, e.g., Student's T test) are said to have inhibited growth. Using reduction of tumor size as an assay, PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 modulators which are capable, e.g., of inhibiting abnormal cell proliferation can be identified.

B. Modulators

The compounds tested as modulators of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein can be any small organic molecule, or a biological entity, such as a protein, e.g., an antibody or peptide, a sugar, a nucleic acid, e.g., an antisense oligonucleotide or a ribozyme, or a lipid. Alternatively, modulators can be genetically altered versions of a PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein. Typically, test compounds will be small organic molecules, peptides, circular peptides, RNAi, antisense molecules, ribozymes, and lipids.

Essentially any chemical compound can be used as a potential modulator or ligand in the assays of the invention, although most often compounds that can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (19933)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, Jan 18, page 33 (1993); isoprenoids, U.S. Pat. No. 5,569,588; thiazolidinones and metathiazanojies, U.S. Pat. No. 5,549,974; pyrrolidines, U.S. Pat. Nos. 5,525,735 and 5,519,134; morpholino compounds, U.S. Pat. No. 5,506,337; benzodiazepines, 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J.; Asinex, Moscow, RU; Tripos, Inc., St. Louis, Mo.; ChemStar, Ltd, Moscow, RU; 3D Pharmaceuticals, Exton, Pa.; Martek Biosciences, Columbia, Md., etc.).

C. Solid State and Soluble High Throughput Assays

In one embodiment the invention provides soluble assays using a PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein, or a cell or tissue expressing a PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein, either naturally occurring or recombinant. In another embodiment, the invention provides solid phase based in vitro assays in a high throughput format, where the PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein or PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 substrate is attached to a solid phase. Any one of the assays described herein can be adapted for high throughput screening.

In the high throughput assays of the invention, either soluble or solid state, it is possible to screen up to several thousand different modulators or ligands in a single day. This methodology can be used for PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 proteins in vitro, or for cell-based or membrane-based assays comprising a PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100- about 1500 different compounds. It is possible to assay many plates per day; assay screens for up to about 6,000, 20,000, 50,000, or more than 100,000 different compounds are possible using the integrated systems of the invention.

For a solid state reaction, the protein of interest or a fragment thereof, e.g., an extracellular domain, or a cell or membrane comprising the protein of interest or a fragment thereof as part of a fusion protein can be bound to the solid state component, directly or indirectly, via covalent or non covalent linkage. A tag for covalent or non-covalent binding can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.). Antibodies to molecules with natural binders such as biotin and appropriate tag binders are also widely available; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

Immunological Detection of PKC-R, PLC-β1, FAK, FAK2, CK2, cMET, FEN1, REV1, APE1, CDK3, PIM1, CDC7L1, CDK7, CNK, PR1;3, STK2 (NEK4), NKIAMRE, or HBO1 Polypeptides

In addition to the detection of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 gene and gene expression using nucleic acid hybridization technology, one can also use immunoassays to detect PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 proteins of the invention. Such assays are useful for screening for modulators of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1, as well as for therapeutic and diagnostic applications. Immunoassays can be used to qualitatively or quantitatively analyze PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein. A general overview of the applicable technology can be found in Harlow & Lane, Antibodies: A Laboratory Manual (1988).

A. Production of Antibodies

Methods of producing polyclonal and monoclonal antibodies that react specifically with the PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 proteins are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

A number of immunogens comprising portions of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein may be used to produce antibodies specifically reactive with PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein. For example, recombinant PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein or an antigenic fragment thereof, can be isolated as described herein. Recombinant protein can be expressed in eukaryotic or prokaryotic cells as described above, and purified as generally described above. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the beta subunits. When appropriately high titers of antibody to the immunogen are obtained, blood is collected from the animal and antisera are prepared. Further fractionation of the antisera to enrich for antibodies reactive to the protein can be done if desired (see, Harlow & Lane, supra).

Monoclonal antibodies may be obtained by various techniques familiar to those skilled in the art. Briefly, spleen cells from an animal immunized with a desired antigen are immortalized, commonly by fusion with a myeloma cell (see, Kohler & Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., Science 246:1275-1281 (1989).

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against nou-PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 proteins, using a competitive binding immunoassay. Specific polyclonal antisera and monoclonal antibodies will usually bind with a K_{d} of at least about 0.1 mM, more usually at least about 1 μM, preferably at least about 0.1 μM or better, and most preferably, 0.01 μM or better. Antibodies specific only for a particular PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 ortholog, such as human PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1, can also be made, by subtracting out other cross-reacting orthologs from a species such as a non-human mammal. In this manner, antibodies that bind only to PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein may be obtained.

Once the specific antibodies against PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein are available, the protein can be detected by a variety of immunoassay methods. In addition, the antibody can be used therapeutically as a PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 modulators. For a review of immunological and immunoassay procedures, see Basic and Clinical Immunology (Stites & Terr eds., 7^{th} ed. 1991). Moreover, the immunoassays of the present invention can be performed in any of several configurations, which are reviewed extensively in Enzyme Immunoassay (Maggio, ed., 1980); and Harlow & Lane, supra.

B. Immunological Binding Assays

PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein can be detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites & Terr, eds., 7th ed. 1991). Irmunological binding assays (or immunoassays) typically use an antibody that specifically binds to a protein or antigen of choice (in this case the PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein or antigenic subsequence thereof). The antibody (e.g., anti-PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1) may be produced by any of a number of means well known to those of skill in the art and as described above.

Immunoassays also often use a labeling agent to specifically bind to and label the complex formed by the antibody and antigen. The labeling agent may itself be one of the moieties comprising the antibody/antigen complex. Thus, the labeling agent may be a labeled PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 or a labeled anti-PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 antibody. Alternatively, the labeling agent may be a third moiety, such a secondary antibody, that specifically binds to the antibody/PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 complex (a secondary antibody is typically specific to antibodies of the species from which the first antibody is derived). Other proteins capable of specifically binding immunoglobulin constant regions, such as protein A or protein G may also be used as the label agent. These proteins exhibit a strong non-immunogenic reactivity with immunoglobulin constant regions from a variety of species (see, e.g., Kronval et al., J. Immunol. 111:1401-1406 (1973); Akerstrom et al., J. Immunol. 135:2589-2542 (1985)). The labeling agent can be modified with a detectable moiety, such as biotin, to which another molecule can specifically bind, such as streptavidin. A variety of detectable moieties are well known to those skilled in the art.

Throughout the assays, incubation and/or washing steps may be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

Non-Competitive Assay Formats

Immunoassays for detecting PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 in samples may be either competitive or noncompetitive. Noncompetitive immunoassays are assays in which the amount of antigen is directly measured. In one preferred “sandwich” assay, for example, the anti-PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 antibodies can be bound directly to a solid substrate on which they are immobilized. These immobilized antibodies then capture PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 present in the test sample. PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 proteins thus immobilized are then bound by a labeling agent, such as a second PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 antibody bearing a label. Alternatively, the second antibody may lack a label, but it may, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

Competitive Assay Formats

In competitive assays, the amount of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein present in the sample is measured indirectly by measuring the amount of a known, added (exogenous) PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein displaced (competed away) from an anti-PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 antibody by the unknown PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein present in a sample. In one competitive assay, a known amount of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein is added to a sample and the sample is then contacted with an antibody that specifically binds to PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein. The amount of exogenous PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein bound to the antibody is inversely proportional to the concentration of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein present in the sample. In a particularly preferred embodiment, the antibody is immobilized on a solid substrate. The amount of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein bound to the antibody may be determined either by measuring the amount of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 present in PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein/antibody complex, or alternatively by measuring the amount of remaining uncomplexed protein. The amount of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein may be detected by providing a labeled PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 molecule.

A hapten inhibition assay is another preferred competitive assay. In this assay the known PKC-ζ, PLC-β1, FAK, FAK2, CK2, cMET, FEN1, REV1, APE1, CDK3, PIM1, CDC7L1, CDK7, CNK., PRL-3, STK2 (NEK4), NKIAMRE, or HBO1 protein is immobilized on a solid substrate. A known amount of anti-PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 antibody is added to the sample, and the sample is then contacted with the immobilized PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1. The amount of anti-PKC-ζ, PLC-β61, FAK, FAK2, CK2, cMET, FEN1, REV1, APE1, CDK3, PIM1, CDC7L1, CDK7, CNK, PRL-3, STK2 (NEK4), NKIAMRE, or HBO1 antibody bound to the known immobilized PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 is inversely proportional to the amount of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

Cross-Reactivity Determinations

Immunoassays in the competitive binding format can also be used for crossreactivity determinations. For example, a PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 can be immobilized to a solid support. Proteins (e.g., PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 and homologs) are added to the assay that compete for binding of the antisera to the immobilized antigen. The ability of the added proteins to compete for binding of the antisera to the immobilized protein is compared to the ability of the PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein to compete with itself. The percent crossreactivity for the above proteins is calculated, using standard calculations. Those antisera with less than 10% crossreactivity with each of the added proteins listed above are selected and pooled. The cross-reacting antibodies are optionally removed from the pooled antisera by immunoabsorption with the added considered proteins, e.g., distantly related homologs.

The immunoabsorbed and pooled antisera are then used in a competitive binding immunoassay as described above to compare a second protein, thought to be perhaps an allele or polymorphic variant of a PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein, to the immunogen protein. In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein that is required to inhibit 50% of binding, then the second protein is said to specifically bind to the polyclonal antibodies generated to PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 immunogen.

Other Assay Formats

Western blot (immunoblot) analysis is used to detect and quantify the presence of PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1. The anti-PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 antibodies specifically bind to the PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 on the solid support. These antibodies may be directly labeled or alternatively may be subsequently detected using labeled antibodies (e.g., labeled sheep anti-mouse antibodies) that specifically bind to the anti-PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 antibodies.

Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev. 5:34-41 (1986)).

Reduction of Non-Specific Binding

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

Labels

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound. The ligands and their targets can be used in any suitable combination with antibodies that recognize PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein, or secondary antibodies that recognize anti-PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by the use of electronic detectors such as charge coupled devices (CCDs) or photomultipliers and the like. Similarly, enzymatic labels may be detected by providing the appropriate substrates for the enzyme and detecting the resulting reaction product. Colorimetric or chemiluminescent labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

Cellular Transfection and Gene Therapy

The present invention provides the nucleic acids of PKC-ζ, PLC-β1, FAK, FAK2, CK2, cMET, FEN1, REV1, APE1, CDK3, PIM1, CDC7L1, CDK7, CNK, PRL-3, STK2 (NEK43), NKIAMRE, or HBO1 protein for the transfection of cells in vitro and in vivo. These nucleic acids can be inserted into any of a number of well-known vectors for the transfection of target cells and organisms as described below. The nucleic acids are transfected into cells, ex vivo or in vivo, through the interaction of the vector and the target cell. The nucleic acid, under the control of a promoter, then expresses a PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein of the present invention, thereby mitigating the effects of absent, partial inactivation, or abnormal expression of a PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 gene, particularly as it relates to cellular proliferation. The compositions are administered to a patient in an amount sufficient to elicit a therapeutic response in the patient. An amount adequate to accomplish this is defined as “therapeutically effective dose or amount.”

Such gene therapy procedures have been used to correct acquired and inherited genetic defects, cancer, and other diseases in a number of contexts. The ability to express artificial genes in humans facilitates the prevention and/or cure of many important human diseases, including many diseases which are not amenable to treatment by other therapies (for a review of gene therapy procedures, see Anderson, Science 256:808-813 (1992); Nabel & Felgner, TIBTECH 11:211-217 (1993); Mitani & Caskey, TIBTECH 11:162-166 (1993); Mulligan, Science 926-932 (1993); Dillon, TIBTECH 11:167-175 (1993); Miller, Nature 357:455-460 (1992); Van Brunt, Biotechnology 6(10):1149-1154 (1998); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology (Doerfler & Böhn eds., 1995); and Yu et al., Gene Therapy 1:13-26 (1994)).

Pharmaceutical Compositions and Administration

Pharmaceutically acceptable carriers are determined in part by the particular composition being administered (e.g., nucleic acid, protein, modulatory compounds or transduced cell), as well as by the particular method used to administer the composition. Accordingly, there are a wide variety of suitable formulations of pharmaceutical compositions of the present invention (see, e.g., Remington's Pharmaceutical Sciences, 17^{th} ed., 1989). Administration can be in any convenient manner, e.g., by injection, oral administration, inhalation, transdermal application, or rectal administration.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration and intravenous administration are the preferred methods of administration. The formulations of commends can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The dose administered to a patient, in the context of the present invention should be sufficient to effect a beneficial therapeutic response in the patient over time. The dose will be determined by the efficacy of the particular vector employed and the condition of the patient, as well as the body weight or surface area of the patient to be treated. The size of the dose also will be determined by the existence, nature, and extent of any adverse side-effects that accompany the administration of a particular vector, or transduced cell type in a particular patient.

In determining the effective amount of the vector to be administered in the treatment or prophylaxis of conditions owing to diminished or aberrant expression of the PKC-ζ, PLC-β1, FAK, FAK2, CK2, cMET, FEN1, REV1, APE1, CDK3, PIM1, CDC7L1, CDK7, CNK, PRL-3, STK2 (NEK4), NKIAMRE, or HBO1 protein, the physician evaluates circulating plasma levels of the vector, vector toxicities, progression of the disease, and the production of anti-vector antibodies. In general, the dose equivalent of a naked nucleic acid from a vector is from about 1 μg to 100 μg for a typical 70 kilogram patient, and doses of vectors which include a retroviral particle are calculated to yield an equivalent amount of therapeutic nucleic acid.

For administration, compounds and transduced cells of the present invention can be administered at a rate determined by the LD-50 of the inhibitor, vector, or transduced cell type, and the side-effects of the inhibitor, vector or cell type at various concentrations, as applied to the mass and overall health of the patient. Administration can be accomplished via single or divided doses.

### EXAMPLES

The following examples are offered to illustrate, but not to limit the claimed invention.

### Example 1

### Identification of Genes That Modulate Cell Proliferation Using Immunoprecipitation Assays

PKCζ, PLCβ1, cMET, PIM1, and NKIAMRE were identified as modulators of cell proliferation using co-immunoprecipitation assays known to those of skill in the art (see, e.g., Harlow and Lane, supra). More specifically, PKCζ, PLCβ1, cMET, PIM1, and NKIAMRE co-immunoprecipitated with cell cycle modulating proteins previously bound to a monoclonal antibody and thus were identified as modulators of cell proliferation. In particular, PKCζ was identified using the monoclonal antibody ATM (specific for a nucleophosphoprotein involved in ataxia telangiectasia); PLCβ1 was identified using the monoclonal antibody p48 (specific for a subunit of the RB tumor suppressor gene); cMET was identified using the monoclonal antibody RbAp48 (specific for a fusion protein corresponding to amino acids 1-425 of human RbAp48); PIM1 was identified using the monoclonal antibody p21 (specific for the tumor suppressor gene p21); and NKIAMRE was identified using the monoclonal antibody RbAp48.

### Example 2

### Identification of Genes That Modulate Cell Proliferation Using Yeast Two Hybrid Assays

FAK, FAK2, CK2, FEN2, REV1, APE1, CDK3, CDC71, CDK7, CNK, PRL-3, STK2 (NEK4), and HBO1 were identified as modulators of cell proliferation using yeast two hybrid assays known to those of skill in the art (see, e.g., Fields and Song, Nature, 340(6230):245 (1989). Briefly, two different haploid yeast strains of opposite mating types (e.g., MATa and MATα) are generated. One strain contains a protein fused to the DNA binding domain (i.e., binds to UASG) of the Saccharomyces cerevisiae transcriptional activator factor GAL4. The GAL4 DNA binding domain is typically placed upstream of reporter genes. Another strain contains a protein fused to the activation domain of GALA. The strains are mated and transcription of the reporter gene is assayed. If the two proteins fused to the GAL4 domains interact to form a protein-protein complex, the DNA binding domain and the activation domain will reconstitute to form a functional transcriptional activator and reporter gene activity will be detected.

### Example 3

### Functional Characterization of Genes that Modulate the Cell Cycle Using Dominant Negative Mutants

Dominant negative mutants are used to study the effects of PKC-ζ, PLC-β1, FAK, FAK2, CK2, cMET, FEN1, REV1, APE1, CDK3, PIM1, CDC7L1, CDK7, CNK, PRL-3, STK2 or NEK4, NKIAMRE, or HBO1 on proliferation, the cell cycle, cell viability, and chemosensitization.

The anti-proliferative effects of dominant negative mutants are determined by GFP positivity assays. Briefly, Cell Tracker (CT) stained cells are infected with retroviruses engineered to express wild type and mutant PKC-ζ, PLC-β1, FAK, FAK2, CK2, cMET, FEN1, REV1, APE1, CDK3, PIM1, CDC7L1, CDK7, CNK, PRL-3, STK2 or NEK4, NKIAMRE, or HBO1. The CT intensity of the GFP expressing population will be compared to the intensity of the GFP negative, uninfected population. Cells that stain brightly with the CT are identified as cell cycle arrested cells. Cells that stain dimly with CT are identified as proliferating cells.

Effects of dominant negative mutants on the cell cycle is measured by DAPI staining of transfected cells.

Effects of dominant negative mutants on cell viability is determined by monitoring the percent of GFP positive cells in an infected population at set intervals following infection.

Effects of dominant negative mutants on chemosensitization is determined by first treating transfected cells with chemotherapeutic agents such as, for example, bleomycin, etoposide, and cisplatin. After treatment with the chemotherapeutic agent, CT assays, DAPI staining assays, and GFP-positivity assays are conducted to assess the effects of PKC-ζ, PLC-β1, FAK, FAK2, CK2, cMET, FEN1, REV1, APE1, CDK3, PIM1, CDC7L1, CDK7, CNK, PRL-3, STK2 or NEK4, NKIAMRE, or HBO1 on proliferation, the cell cycle, cell viability, and chemosensitization.

Dominant negative mutants are used to determine the effects of PKC-ζ, PLC-β1, FAK, FAK2, CK2, cMET, FEN1, REV1, APE1, CDK3, PIM1, CDC7L1, CDK7, CNK, PRL-3, STK2 or NEK4, NKIAMRE, or HBO1 in different tumor types such as, for example, lung, colon, cervical, liver, kidney, uterine, or breast. Exemplary tumor cells lines include, A549 cells (lung, p53 wt), H1299 (lung, p53 null), Hela (cervix, p53 deficient), Colo205 (colon, p53 mutant), and HCT116 (colon, p53 wt).

Dominant negative mutants are also used to determine the effects of PKC-ζ, PLC-β1, FAK, FAK2, CK2, cMET, FEN1, REV1, APE1, CDK3, PIM1, CDC7L1, CDK7, CNK, PRL-3, STK2 or NEK4, NKIAMRE, or HBO1 in tumor cells versus normal cells. Exemplary tissue types include mammary epithelial cells, prostate epithelial cells, lung cells, kidney cells, cervical cells and colon cells.

Dominant negative mutants were generated for CDC7L1, CNK, STK2, Hbo1, PIM1, APE1, CK2 or CK2α, NKIAMRE, FEN1, and CDK3. The results are described in examples below.

### Example 4

### Functional Characterization of Genes that Modulate the Cell Cycle Using siRNA

Short interfering RNAs (siRNAs) are used to study the effects of PKC-ζ, PLC-β1, FAK, FAK2, CK2, cMET, FEN1, REV1, APE1, CDK3, PIM1, CDC7L1, CDK7, CNK, PRL-3, STK2 or NEK4, NKIAMRE, or HBO1 on proliferation and chemosensitization.

Four siRNAs are designed for each gene and transfected into A549 cells and Hela cells. mRNA reduction is tested using Taqman. siRNAs that induce greater than 70% mRNA reduction are tested for anti-proliferative effects. Cy-3 labeled control siRNA, scrambled siRNAs, and the transfection reagent are used as controls.

siRNAs which show no independent anti-proliferative effects are analyzed for their ability to confer chemosensitization. 48 hours post transfection, cells are treated with chemotherapeutic agents, such as, for example, bleomycin, etoposide, and cisplatin. 48 hours post-treatment, the IC50 of each chemotherapeutic agent is determined using BrdU ELISA and/or Cellomics image analysis which counts colonies and measures colony size.

siRNAs were designed for CDC7L1, CNK, Hbo1, PIM1, CK2 or CK2α, and NKIAMRE. The results are discussed in examples below.

### Example 5

### Functional Characterization of Genes that Modulate the Cell Cycle Using Antisense Oligonucleotides

Antisense oligonucleotides are used to study the effects of PKC-ζ, PLC-β1, FAK, FAK2, CK2, cMET, FEN1, REV1, APE1, CDK3, PIM1, CDC7L1, CDK7, CNK, PRL-3, STK2 or NEK4, NKIAMRE, or HBO1 on proliferation and chemosensitization. Briefly, antisense oligonucleotides with a mixed phosphothiorate backbone are used to transfect A549 and Hela cells. Oligonucleotide concentrations of 50 nM or 100 nM are used to transfect the cells. Oligonucleotides which induce greater than 70% mRNA reduction in transfected cells will be tested for anti-proliferative effects. Cell proliferation and viability assays are performed 48 hours post transfection with a BrdU ELISA and/or Cellomics image analysis which counts colonies and measures colony size. Antisense oligonucleotides which show no independent anti-proliferative effects are analyzed for their ability to confer chemosensitization. 48 hours post transfection, cells are treated with chemotherapeutic agents, such as, for example, bleomycin, etoposide, and cisplatin. 48 hours post-treatment, the IC50 of each chemotherapeutic agent is determined using BrdU ELISA and/or Cellomics image analysis.

Antisense oligonucleotides are used to determine the effects of PKC-ζ, PLC-β1, FAK, FAK2, CK2, cMET, FEN1, REV1, APE1, CDK3, PIM1, CDC7L1, CDK7, CNK, PRL-3, STK2 or NEK4, NKIAMRE, or HBO1 in different tumor types such as, for example, lung, colon, cervical, liver, kidney, uterine, or breast. Exemplary tumor cells lines include, A549 cells (lung, p53 wt), H1299 (lung, p53 null), Hela (cervix, p53 deficient), Colo205 (colon, p53 mutant), and HCT115 (colon, p53 wt).

Antisense oligonucleotides are also used to determine the effects of PKC-ζ, PLC-β1, FAK, FAK2, CK2, cMET, FEN1, REV1, APE1, CDK3, PIM1, CDC7L1, CDK7, CNK, PRL-3, STK2 or NEK4, NKIAMRE, or HBO1 in tumor cells versus normal cells. Exemplary tissue types include mammary epithelial cells, prostate epithelial cells, lung cells, kidney cells, cervical cells and colon cells.

### Example 6

### Identification of Genes that Modulate the Cell Cycle Using Proteomics

Proteomics assays are used to identify proteins that bind to PKC-ζ, PLC-β1, FAK, FAK2, CK2, cMET, FEN1, REV1, APE1, CDK3, PIM1, CDC7L1, CDK7, CNK, PRL-3, STK2 or NEK4, NKIAMRE, or HBO1. Typically, the proteomics assays are performed after a functional screen to identify a gene of interest. Briefly, a potential binding partner is mixed with a PKC-ζ, PLC-β1, FAK, FAK2, CK2, cMET, FEN1, REV1, APE1, CDK3, PIM1, CDC7L1, CDK7, CNK, PRL-3, STK2 or NEK4, NKIAMRE, or HBO1 polypeptide bound to an affinity tag (i.e. a labeled monoclonal antibody). Complexes of the potential binding partner bound to the polypeptide are extracted, and analyzed, and the potential binding partner is identified.

### Example 7

### Assay for PLCβ1 Activity

PLCβ1 activity can be measured according to the method described in Nomoto et al., Jpn. J Canc. Res., 89:1257-1266 (1998). Briefly, cell extracts are prepared and an appropriate amount of cell extract is suspended in reaction buffer (50 mM HEPES, pH 7.0, 100 mM NaCl, 1 mM CaCl₂, 0.15 mg/ml bovine serum albumin, and 1 mg/ml sodium deoxycholate) mixed with micelles of a substrate mixture of 1-α-phosphatidyl inositol and 1-α-phosphatidyl [2-³H] inositol or a substrate mixture of 1-α-phosphatidyl inositol 4, 5-biphosphate and 1-α-phosphatidyl [2-³H] inositol 4,5-biphosphate at final concentrations of 100 μM and 10⁴ dpm, respectively. After an appropriate incubation, the reaction is stopped, lipids are extracted from the reaction mixture and radioactivity in the aqueous fraction is detected with a liquid scintillation counter. Percent degradation of the labeled substrate is indicative of enzymatic activity.

### Example 8

### Assay for FAK2 Activity

FAK2 protein-tyrosine kinase activity can be measured according to the method described in Sasaki et al., J. Bio. Chem., 270(6):21206 (1995). Briefly, clarified cell lysates are incubated in 20 μl of kinase assay buffer with 5 μg/20 μl of poly (Glu,Tyr), 5 μCi of [γ-³²P]ATP, 5 μM unlabeled ATP, and 5 M MgCl₂. After an appropriate incubation, the reaction is stopped, and labeled substrate is separated by SDS-PAGE. ³²P-phosphorylated poly (Glu,Tyr) is visualized and quantitated by bioimaging analysis.

### Example 9

### Assay for CK2 Activity

CK2 activity can be measured according to the method described in Messenger et al., J. Biol. Chem., 277(25):23054 (2002). Briefly, cell extracts are incubated in 1 mM of a synthetic peptide substrate, RRRDDDSDDD in 20 mM Tris-HCl pH 7.5, 60 mM NaCl, 10 mM MgCl₂, 1 mM DTT, and 100 μM γ-32P-ATP. After an appropriate incubation, the reactions are stopped, run on SDS-PAGE, and phosphorylated proteins are detected by bioimaging analysis.

### Example 10

### Assay for cMET Activity

cMET activity can be measured according to the method described in Jeffers et al., Proc. Nat 7. Acad. Sci. USA 94:11445 (1997). Briefly, cell lysates are prepared and immunoprecipitated using anti-Met SP260 (Santa Cruz Biotechnology) monoclonal antibody. Immunoprecipitates are assessed or tyrosine kinase activity toward the exogenous substrate gastrin using a tyrosine kinase assay kit from Boehringer Mannheim.

### Example 11

### Assay for FEN1 Activity

FEN1 activity can be measured according to the method described in Tom et al., J. Biol. Chem. 275(14):10498 (2000). Briefly, FEN1 is purified from cell extracts and incubated with appropriate amounts of oligonucleotide substrates and proliferating cell nuclear antigen in reaction buffer (30 mM HEPES pH 7.6, 5% glycerol, 40 mM KCL, 0.1 mg. ml bovine serum albumin, and 8 mM MgCl₂). After an appropriate incubation, the reactions are stopped, run on SDS-PAGE, and products are detected by bioimaging analysis.

### Example 12

### Assay for REV1 Activity

REV1 activity can be measured according to the method described in Zhang et al., Nuc. Acids Res. 30(7):1630 (2002)). Briefly, REV1 is purified from cell extracts and incubated in reaction buffer (25 mM KH₂PO₄ pH 7.0, 5 mM MgCl₂, 10% glycerol, and 50 μM of dNTPs (dATP, dCTP, dTTP, and dGTP) and 50 fmol of a DNA substrate containing a 5′-³²p labeled primer. After an appropriate incubation, the reactions are stopped, run on SDS-PAGE, and products are detected by bioimaging analysis.

### Example 13

### Assay for APE1 Activity

APE1 activity can be measured according to the method described in Tom et al., J. Biol. Chem., 276(52):48781 (2001). Briefly, APE1 is purified from cell extracts and incubated with appropriate amounts of oligonucleotide substrates in reaction buffer (30 mM HEPES pH 7.6, 5% glycerol, 40 mM KCL, 0.01% Nonidet P-40, 1 mg/ml bovine serum albumin, 8 mM MgCl₂, and 0.1 mM ATP). After an appropriate incubation, the reactions are stopped, run on SDS-PAGE, and products are detected by bioimaging analysis.

### Example 14

### Assay for CDC7 L1 Activity

CDC7L1 activity can be measured according to the method described in Masai, et al., J. Biol. Chem., 275(37):29042 (2000). Briefly CDC7L1-ASK complexes are purified, mixed with [γ-32P]ATP (1 μCi) and added to a reaction mixture containing MCM2-4-6-7-previously incubated with cdks and p27. After an appropriate incubation, the reactions are stopped, run on SDS-PAGE, and products are detected by bioimaging analysis.

### Example 15

### Assay for CNK Activity

CNK activity can be measured according to the method described in Ouyang et al., J. Biol. Chem. 274:28646 (1997). Briefly, CNK is purified and assayed for kinase activity using one or more of the following substrates: casein (15 μg/reaction), p53, GST-Cdc25A (5 μg/reaction), GST-Cdc25B (5 μg/reaction), His6-Cdc25c (5 μg/reaction), GST-Cdc25C (1 μg/reaction), or GST-Cdc25C^{S216A} (1 μg/reaction).

### Example 16

### Assay for STK2 (NEK4) Activity

STK2 (NEK4) activity can be measured according to the method described in Hayashi et al., Biochem. Biophys. Res. Comm., 264:449 (1999). Briefly, STK2 complexes are immunoprecipitated, resuspended in kinase buffer (50 mM Tris-HCl pH 7.2, 3 mM MnCl₂) containing 10 μCi [γ-32P]ATP and 5 μg of exogenous protein substrates. After an appropriate incubation, the reactions are stopped, the phosphorylated proteins are separated by SDS-PAGE, and detected by bioimaging analysis.

### Example 17

### Assay for HBO1 Activity

HBO1 can be measured according to the method described in Iiuzuka and Stilman, J. Bio. Chem., 274(33):23027 (1999). Briefly, HBO1 polypeptides are immunoprecipitated from cell extracts and combined with a mixture recombinant Xenopus histone H3₂.H4₂ tetramers (100 μg/ml), human histone H2A.H2B (100 μg/ml), and pmol of [³H]acetyl coenzyme A (11.2 Ci/mmol) in an appropriate volume of assay buffer (25 mM Tris-HCl, ph 8.5m 1 mM dithiothreitol, 0.5 mM EDTA, 5 mM sodium butyrate, 150 mM NaCl, 10% glycerol). After an appropriate incubation, the reactions are stopped, the phosphorylated proteins are separated by SDS-PAGE, and detected by Coomassie blue staining.

### Example 18

### Functional Characterization of CDC7 L1 Using Dominant Negative Mutants and siRNA Assays

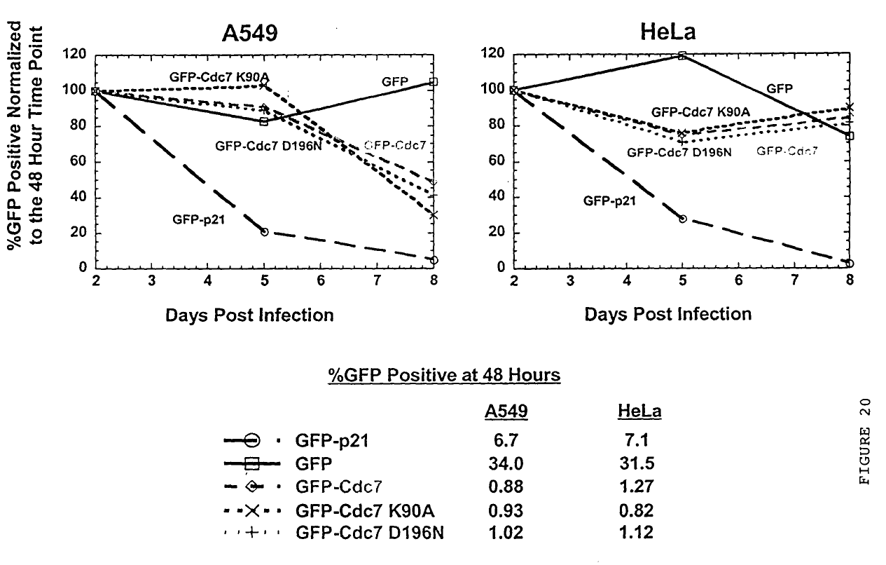

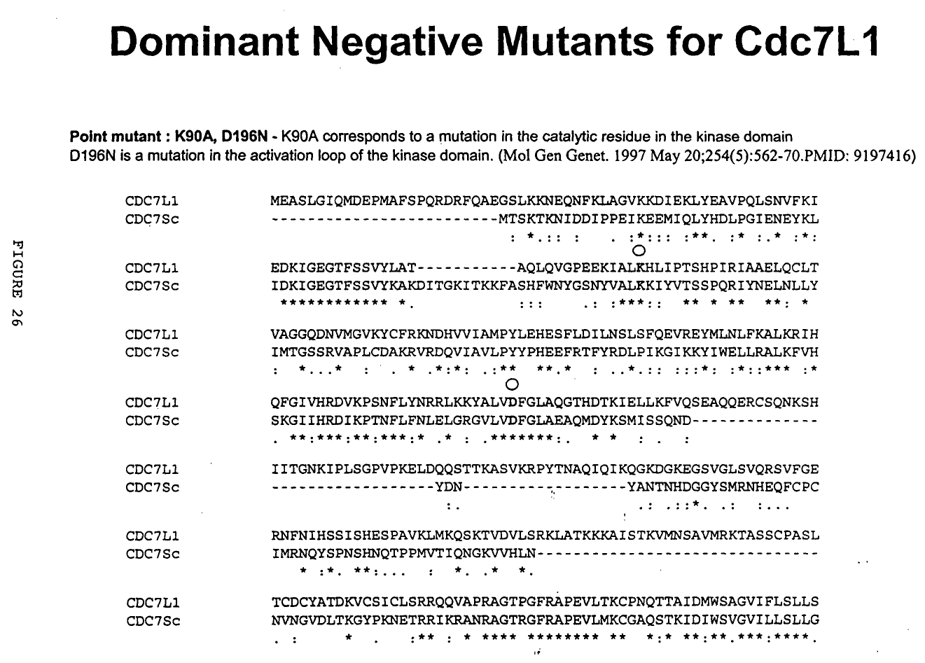

CDC7LI was identified as a modulator of cellular proliferation in a yeast two hybrid assay using apoptin and GADD45. Vectors for the expression of CDC7LI fused to the C-terminus of GFP with a tetOff inducible gene expression system were used to transfect A549 cells and Hela cells. Cell proliferation was measured using Cell Tracker assays, i.e., detecting GFP positivity. As shown in FIG. 20, expression of wild-type GFP-CDC7 LI and mutant GFP-CDC7LI inhibited proliferation of A549 cells. The amino acid sequence of CDC7L muntants is shown in FIG. 26.

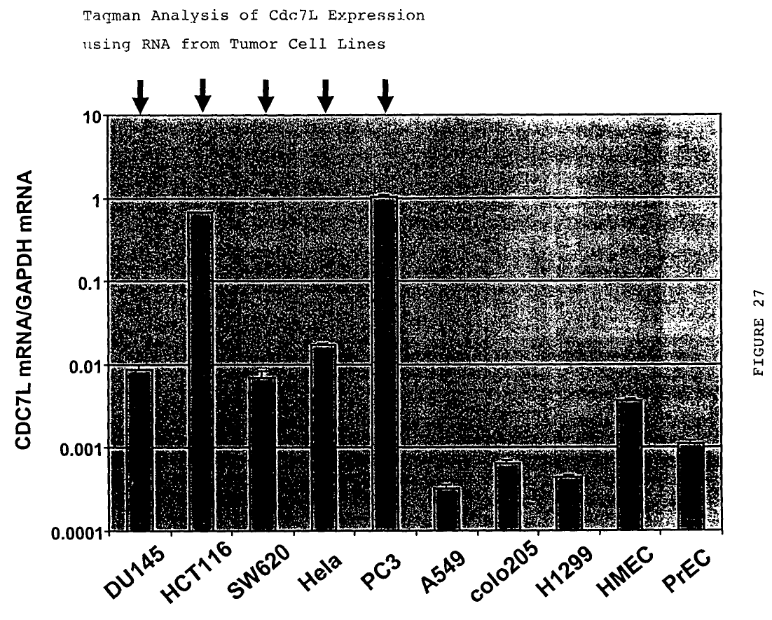

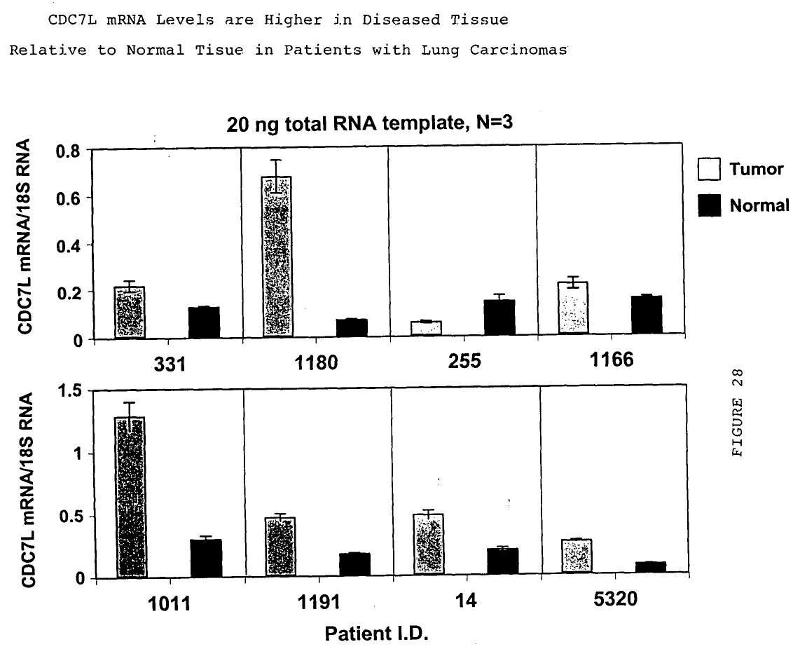

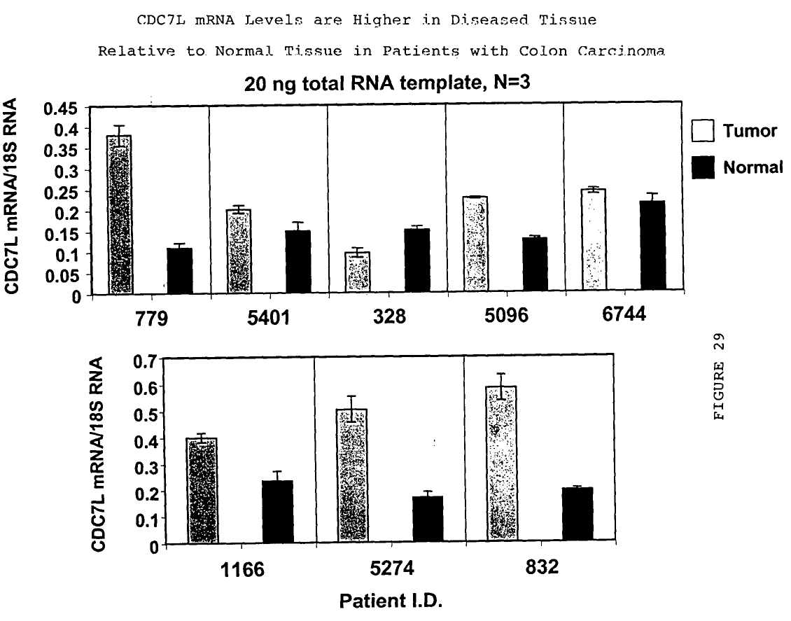

CDC7LI mRNA expression was analyzed in tumor cell lines and in lung carcinomas and colon carcinomas. CDC7LI mRNA was overexpressed in tumor cell lines (e.g., DU145, HCT116, SW620, Hela, and PC3) as compared to primary cell lines. See, e.g., FIG. 27. FIG. 28 demonstrates that CDC7LI mRNA is expressed at higher levels in some lung carcinomas compared to normal tissue from the same patient. FIG. 29 demonstrates that CDC7LI mRNA is expressed at higher levels in some colon carcinomas compared to normal tissue from the same patient.

Two siRNAs induced greater than 50% reduction in mRNA expression when transfected into Hela cells. One of these siRNAs induced greater than 70% reduction in mRNA expression. (Data not shown.)

### Example 19

### Functional Characterization of CNK Using Dominant Negative Mutants and siRNA Assays

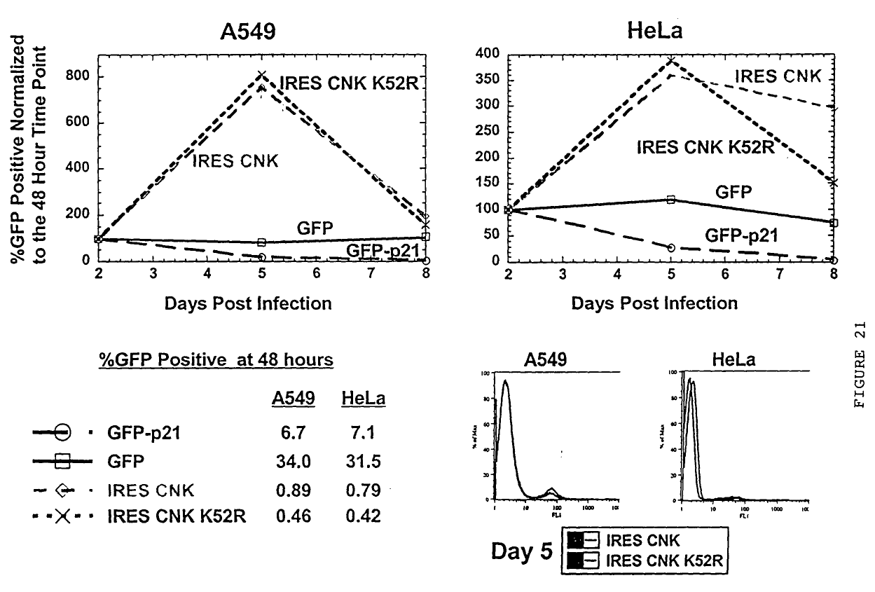

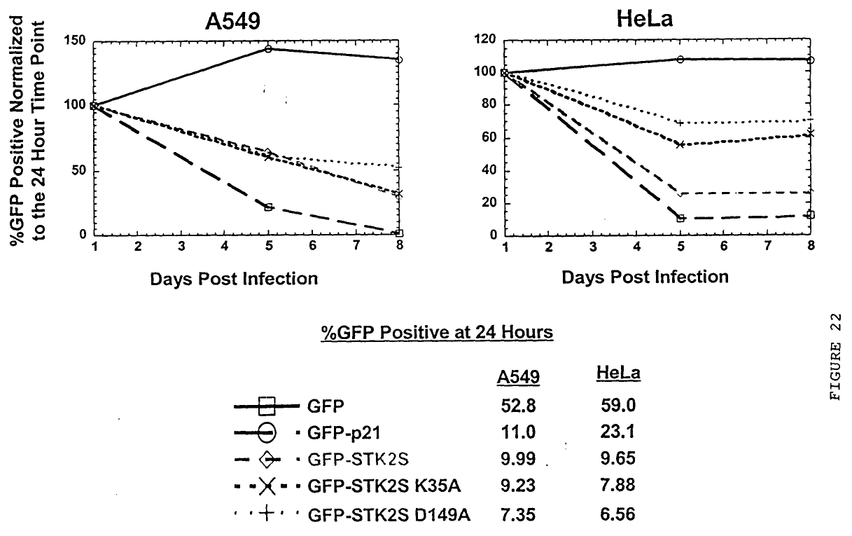

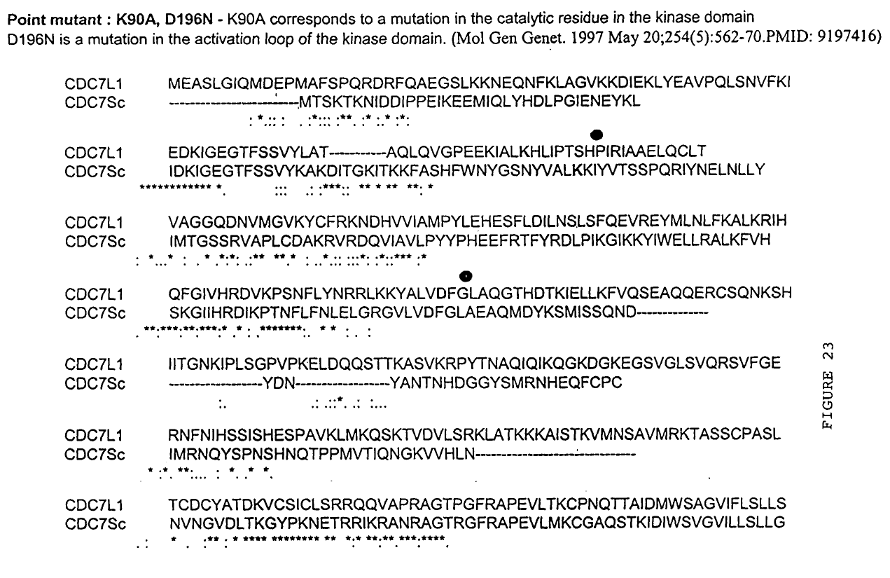

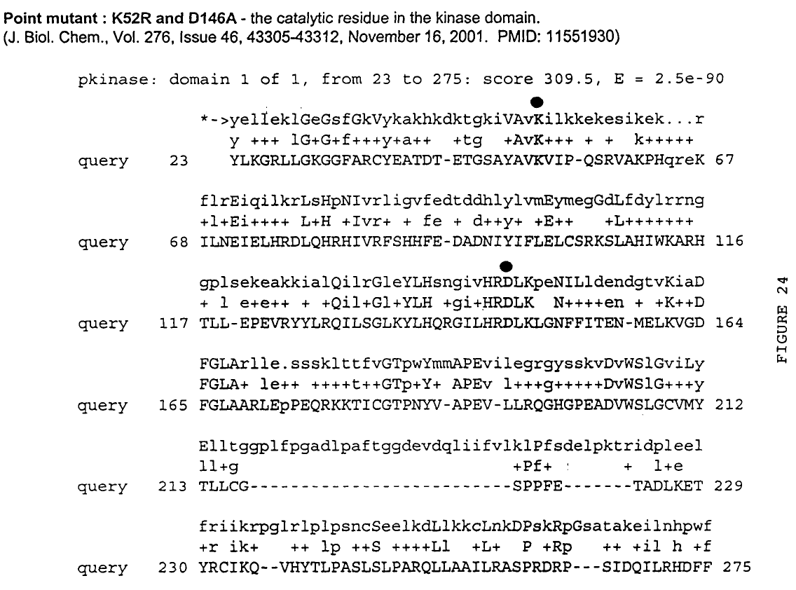

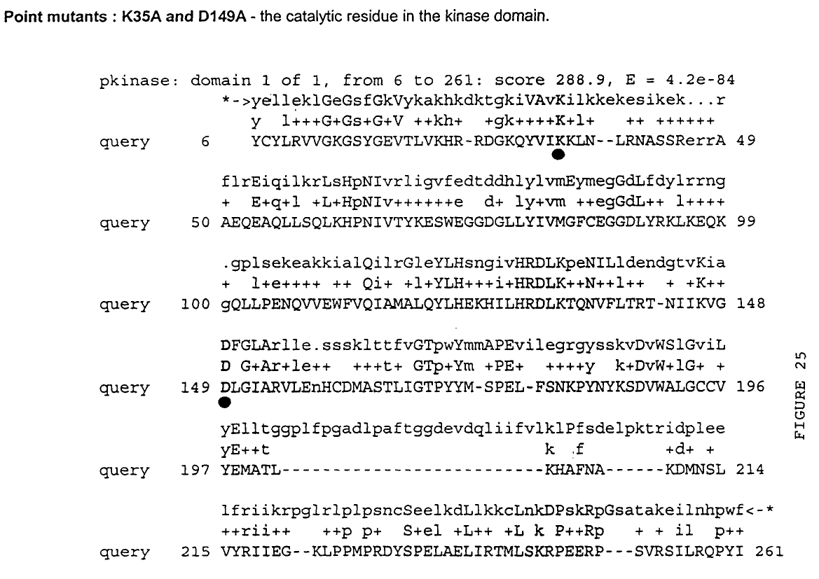

CNK was identified as a modulator of cellular proliferation in a yeast two hybrid assay using DNAPK and F10. Vectors for the expression of CNK fused to the C-terminus of GFP with a tetOff inducible gene expression system were used to transfect A549 cells and Hela cells. Cell proliferation was measured using Cell Tracker assays, i.e., detecting GFP positivity. As shown in FIG. 21, expression of wild-type CNK and mutant GFP-CNK inhibited proliferation of A549 cells. None of the siRNAs tested induced greater than 50% reduction in mRNA expression.

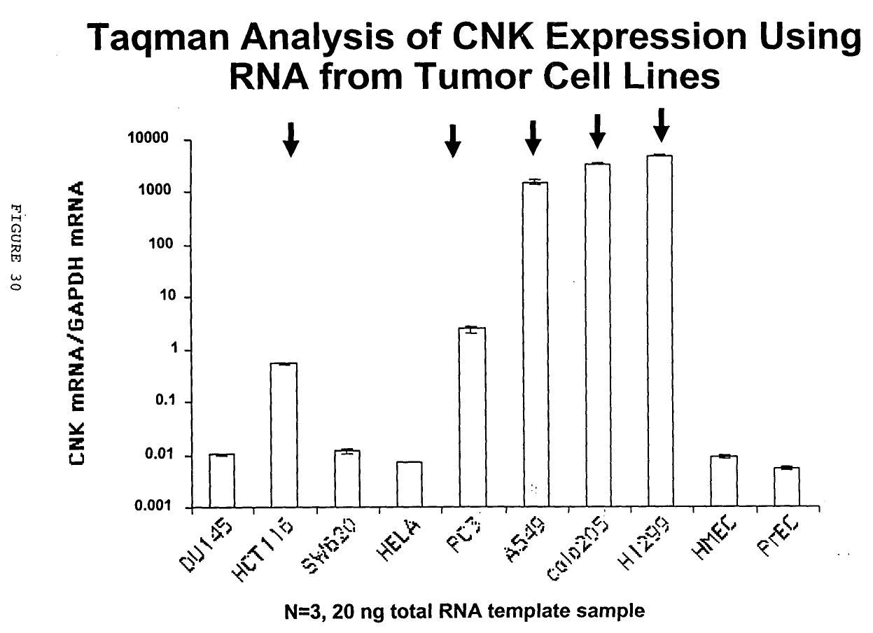

CNK mRNA expression was analyzed in tumor cell lines. CNK mRNA was overexpressed in tumor cell lines (e.g., HCT116, PC3, A549, colo205, and H1299) as compared to primary cell lines. See, e.g., FIG. 30.

Wild type CNK and the CNK D146A mutant were fused to GST and produced in E. coli. (Data not shown.) Briefly, BL21(DE3) cells were transformed with either pDEST15-CNK WT or CNK D146A and grown at 37° C. to an OD600 of 0.6. Cultures were induced with 1 mM IPTG and then transferred to a 16° C. shaking incubator for overnight incubation. After immobilization on glutathione-sepharose, proteins were eluted with 7.5 mM glutathione. The yield was approximately 0.5 mg/L for each protein.

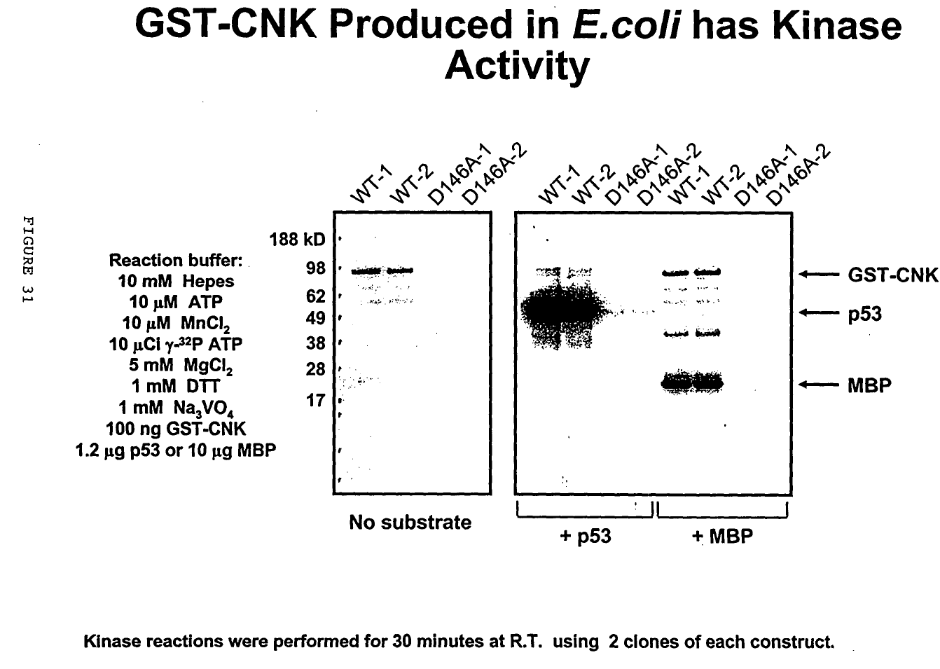

The GST CNK fusions were tested for kinase activity in duplicate assays. See, e.g., FIG. 31. The reaction buffer contained the following components: Reaction buffer: 10 mM Hepes, 10 μM ATP, 10 μM MnCl₂, 10 μCi γ-³²P ATP, 5 mM MgCl₂, 1 mM DTT, 1 mM Na₃VO₄, 100 ng GST-CNK, 1.2 μg p53 or 10 μg MBP. Kinase reactions were incubated for thirty minutes at room temperature. The GST-CNK D146A mutant did not exhibit kinase activity. Wild type GST-CNK phosphorylated p53, maltose binding protein (MBP) and also exhibited autophosphorylation activity.

### Example 20

### Functional Characterization of STK2 Using Dominant Negative Mutants

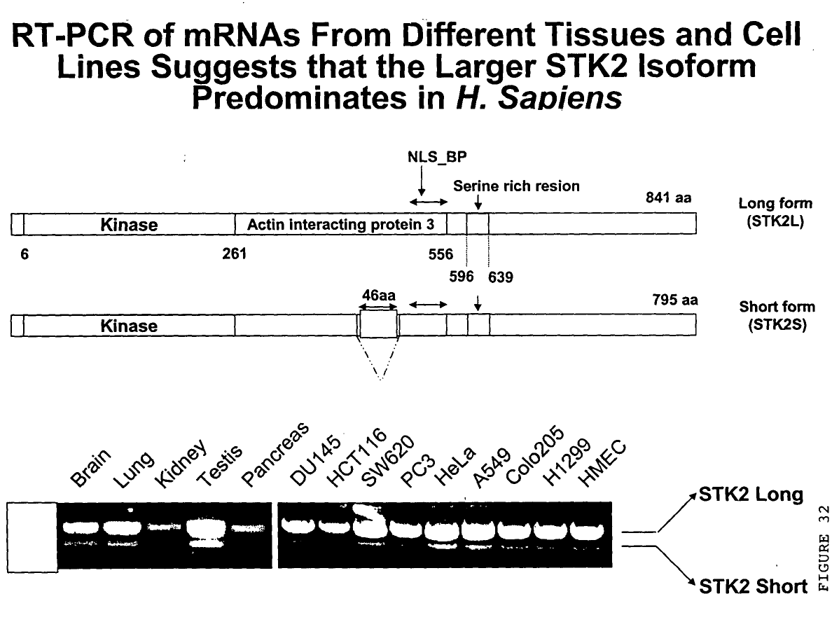

STK2 was identified as a modulator of cellular proliferation in a yeast two hybrid assay using p73. STK2 is expressed as long and short isoforms (STK2L and STK2S). STK2L appears to be more highly expressed than STK2S in humans. See, e.g., FIG. 32.

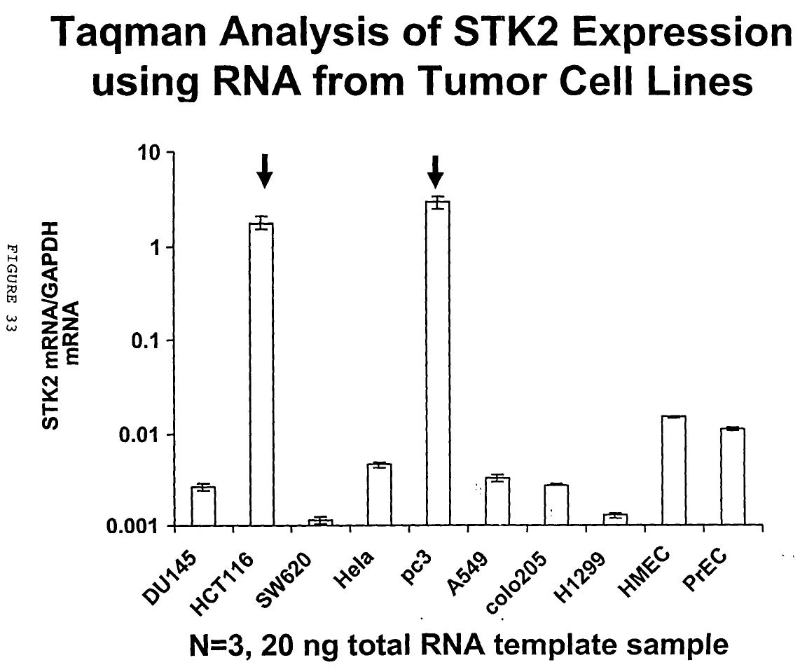

STK2 mRNA expression was analyzed in tumor cell lines. STK2 mRNA was overexpressed in tumor cell lines (e.g., HCT116 and PC3) as compared to primary cell lines. See, e.g., FIG. 33.

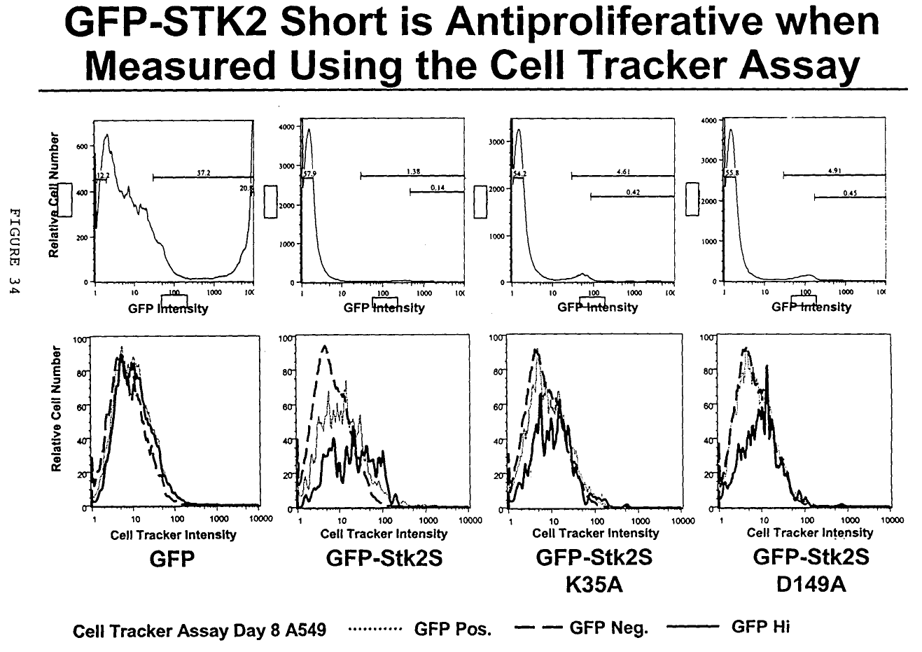

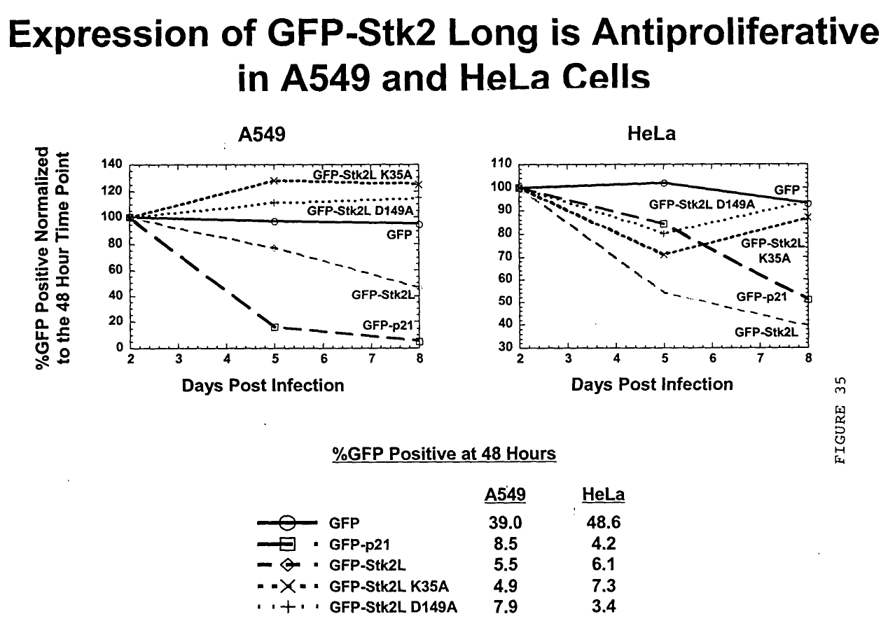

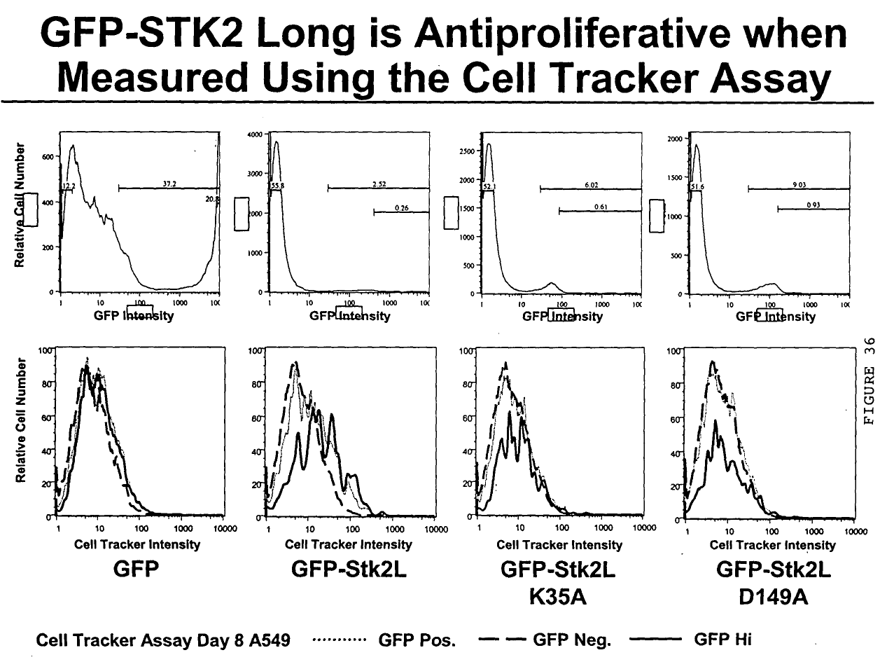

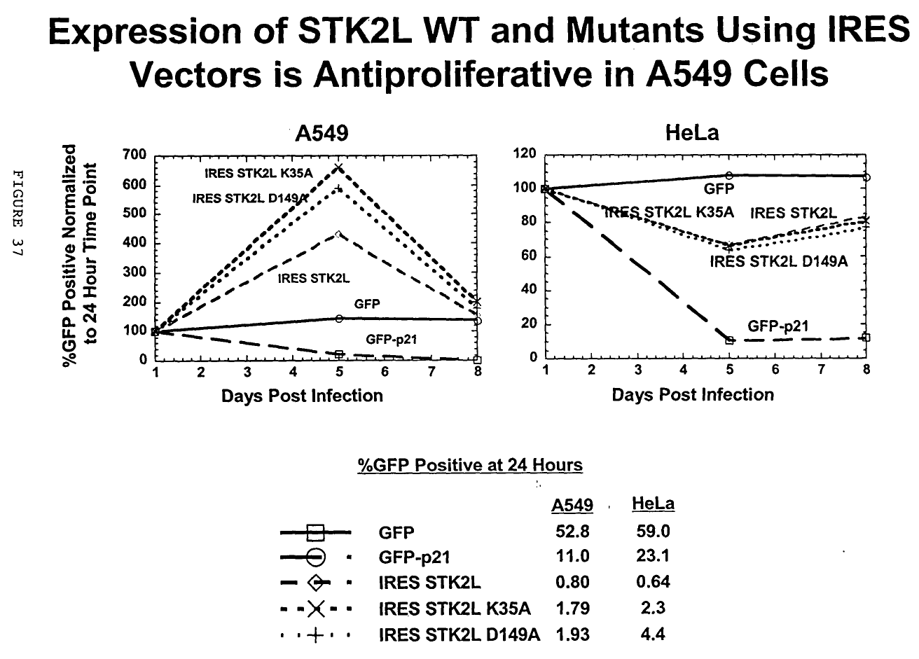

STK2 clones from a GFP C-terminal cDNA fusion library with a tetOff inducible gene expression system were used to transfect A549 cells and Hela cells. Cell proliferation was measured using Cell Tracker assays, i.e., detecting GFP positivity. As shown in FIG. 22, expression of wild-type STK2S inhibited proliferation of A549 cells and in Hela cells and expression of and mutant STK2S inhibited proliferation of A549 cells. Similar results are shown in FIG. 34. FIG. 35 shows that expression of GFP-STK2L inhibited proliferation of A549 and HeLa cells. Similar results were obtained for STK2L as shown in FIG. 36. Using IRES vectors, expression of STK2L wild type and mutant proteins inhibited proliferation in A549 cells. See, e.g., FIG. 37.

### Example 21

### Functional Characterization of HBO1

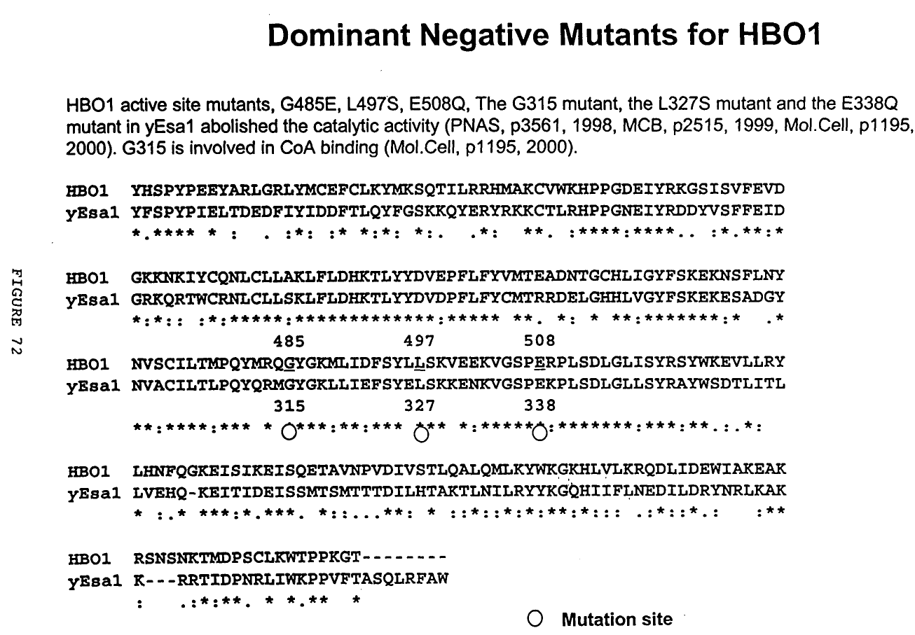

Hbo1 mutants were constructed with the following mutations: Hbo1 G484E, Hbo1 L497S, and Hbo1 E508Q. Hbo1 mutants are shown in FIG. 72. Both wild type and mutant Hbo1 proteins were localized to the cell nucleus. (Data not shown.)

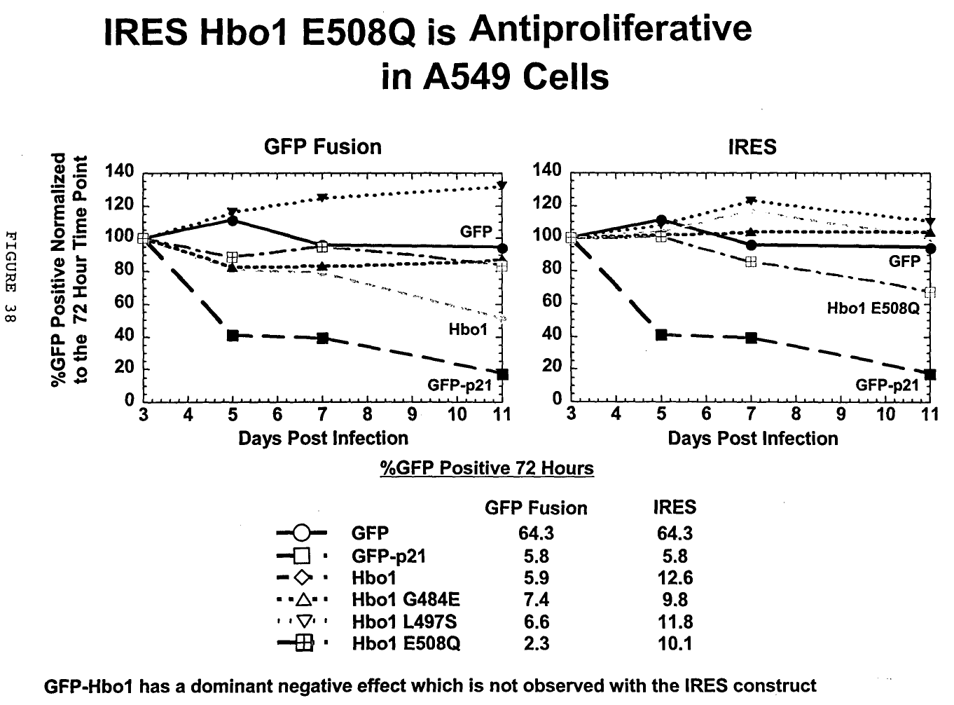

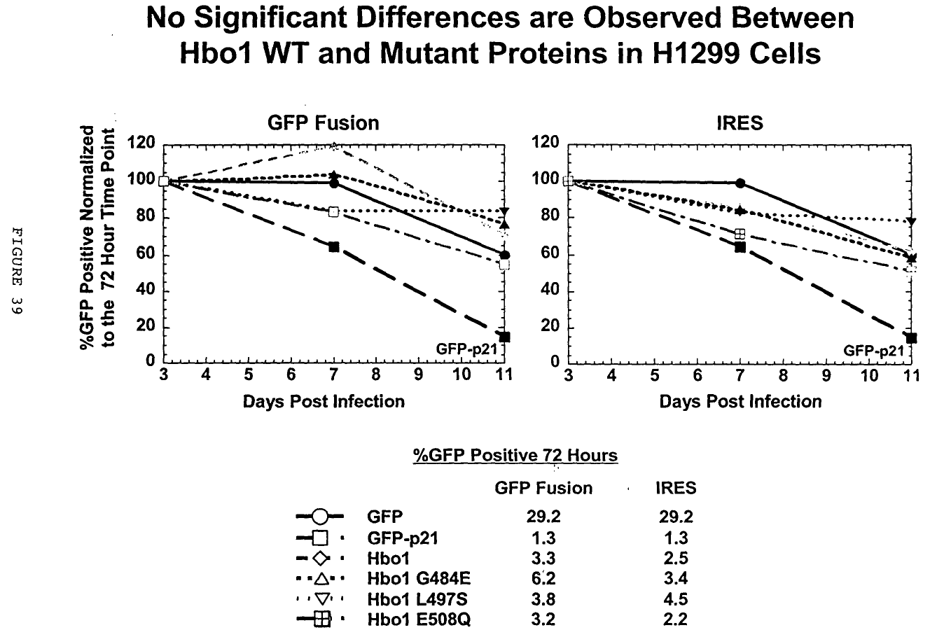

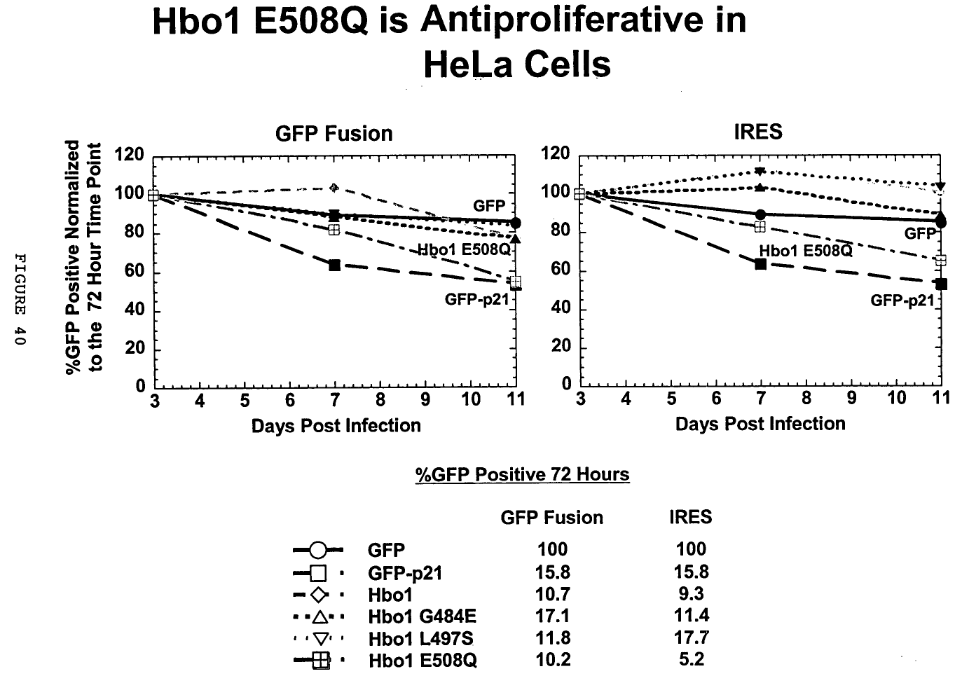

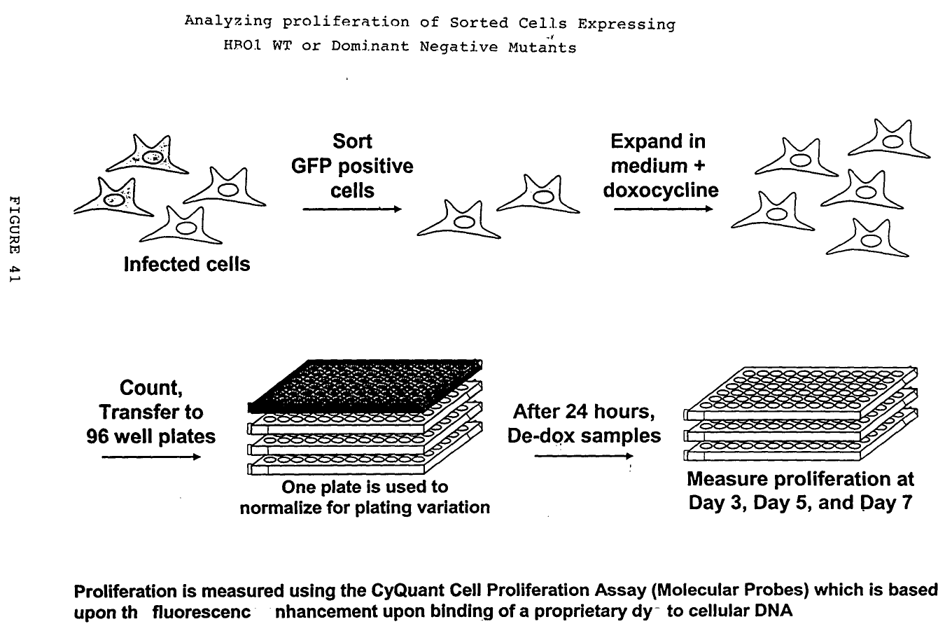

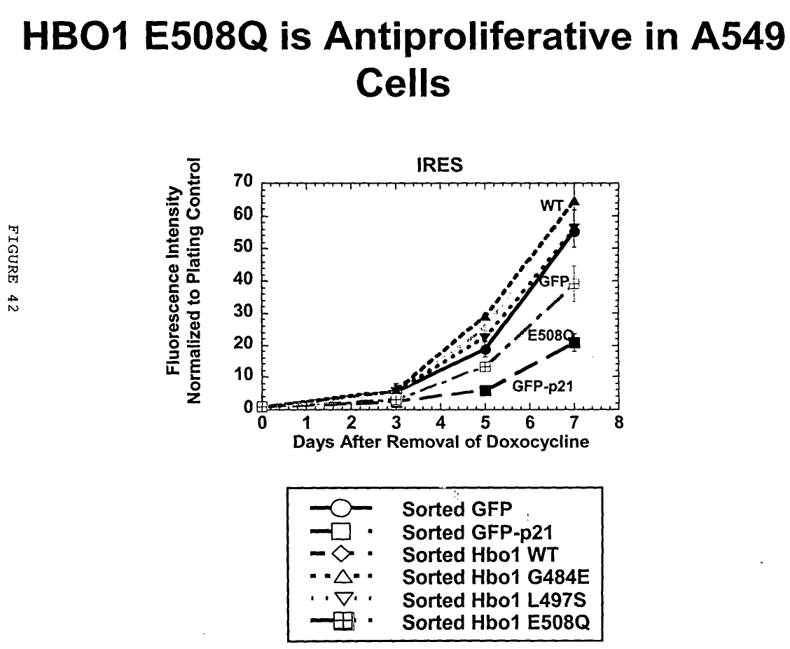

The effect of Hbo1 expression on tumor cell lines was determined using cells that had been infected with a retrovirus that expressed HBO1 wild type or mutant proteins. The Hbo1 E508Q mutant was antiproliferative in A549 cells (IRES only) and HeLa cells (GFP fusion and IRES construct) and had no effect in H1299 cells. Expression of the wild type Hbo1 protein and the other mutants had no effect on proliferation in this assay. See, e.g., FIGS. 38-40. Additional assays were performed using only sorted GFP positive cells as shown in FIG. 41. Proliferation was measured using the CyQuant Cell Proliferation Assay (Molecular Probes) which is based upon the fluorescence enhancement upon binding of a proprietary dye to cellular DNA. Using sorted cells, the Hbo1 E508Q mutant was strongly antiproliferative in A549 cells and HeLa cells. See, e.g., FIGS. 42-43.

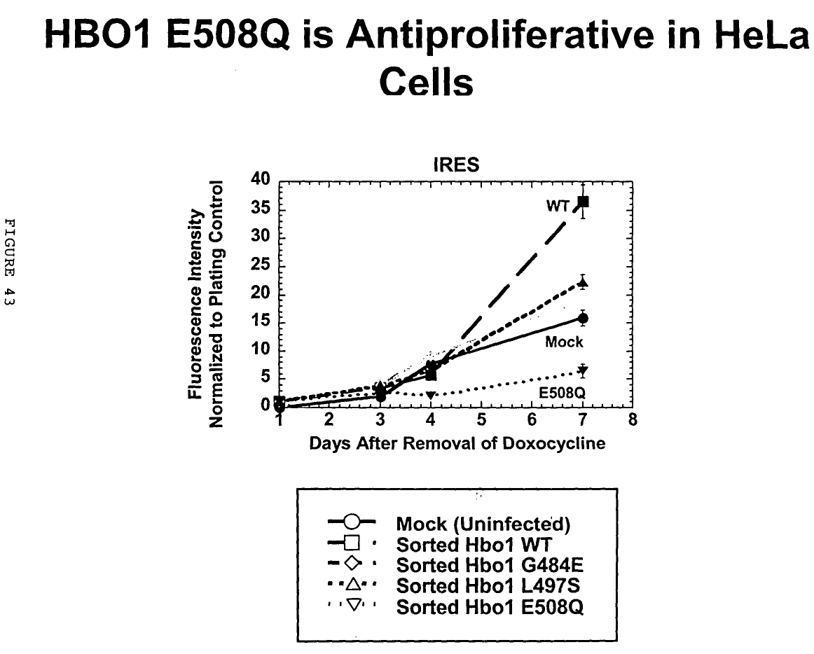

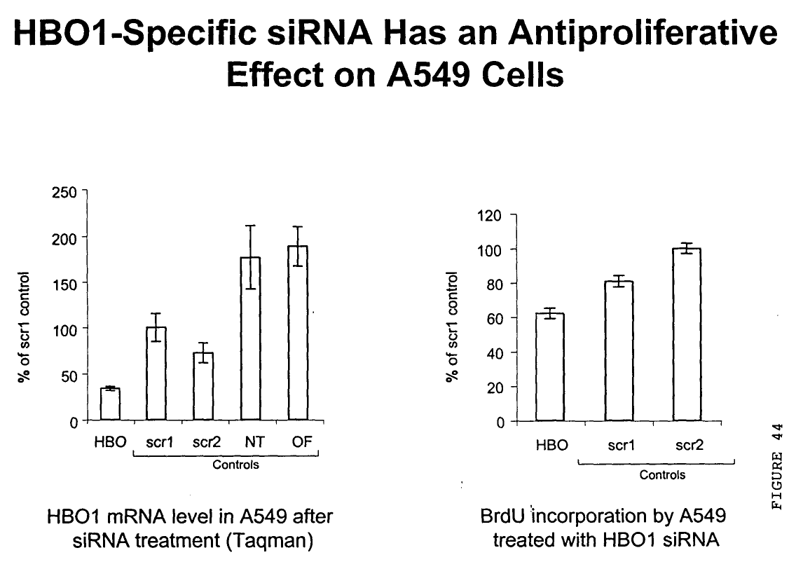

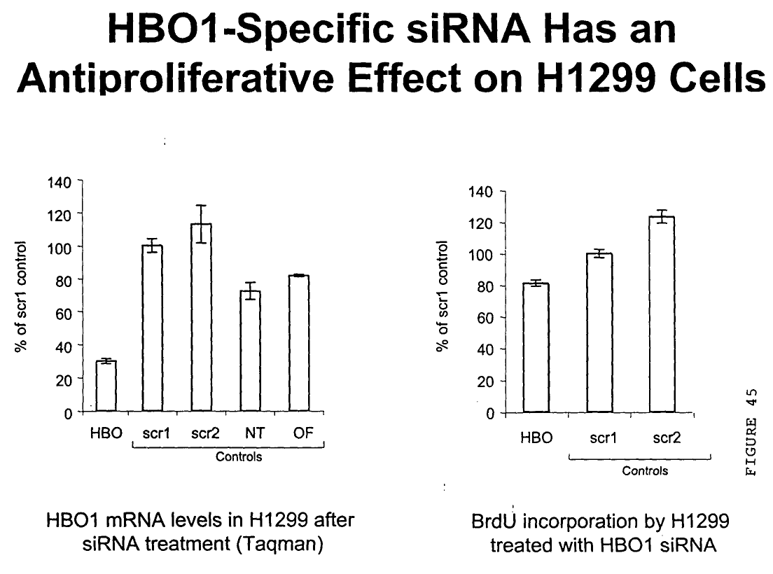

An Hbo1 siRNA caused greater than 50% reduction in mRNA expression when transfected into A549 cells or H1299 cells. The sequence of the Hbo1 siRNA is as follows: AACTGAGCAAGTGGTTGATTT. The Hbo1 siRNA had an antiproliferative effect when expressed in A549 or H1299 cells. See, e.g., FIGS. 44-45.

### Example 22

### Functional Characterization of PIM1

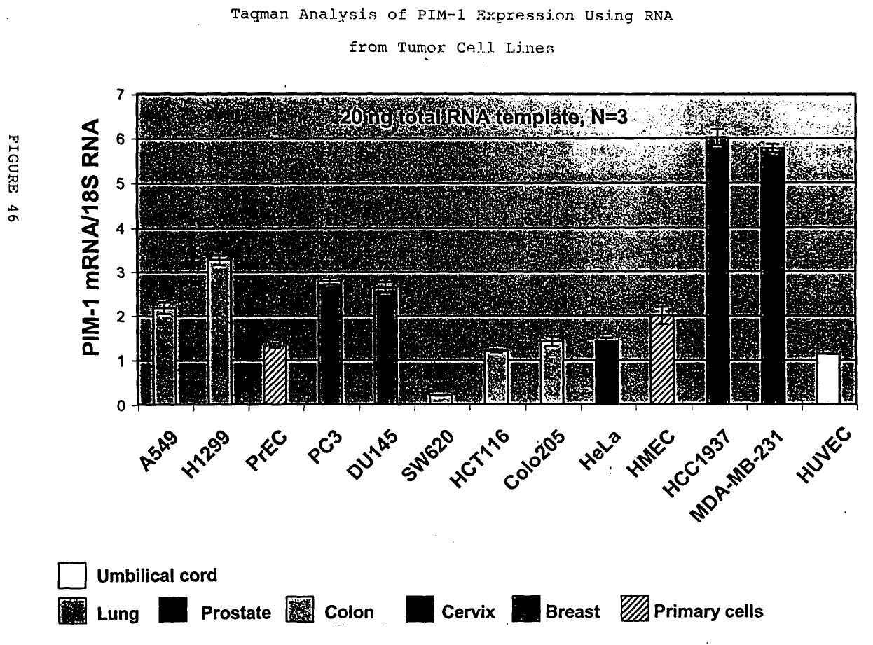

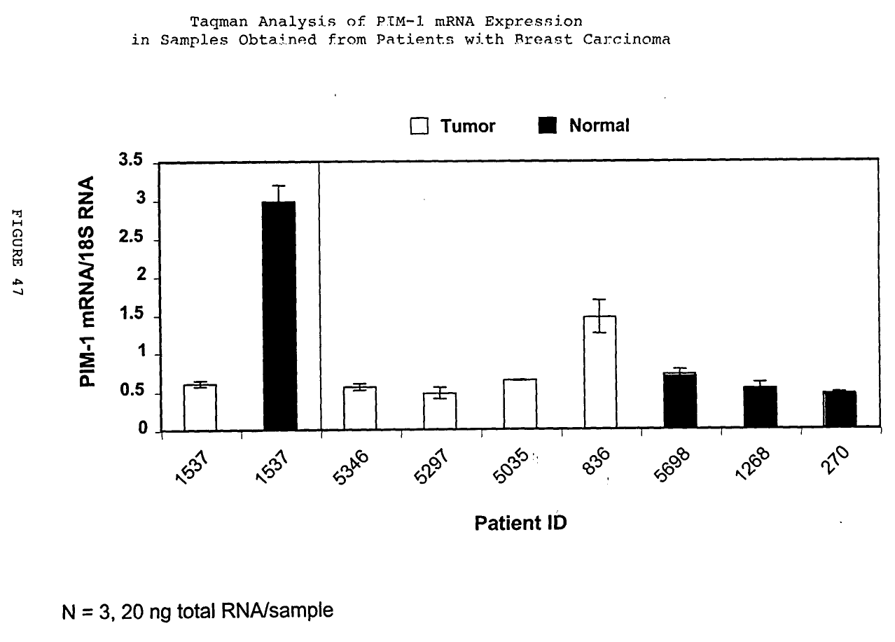

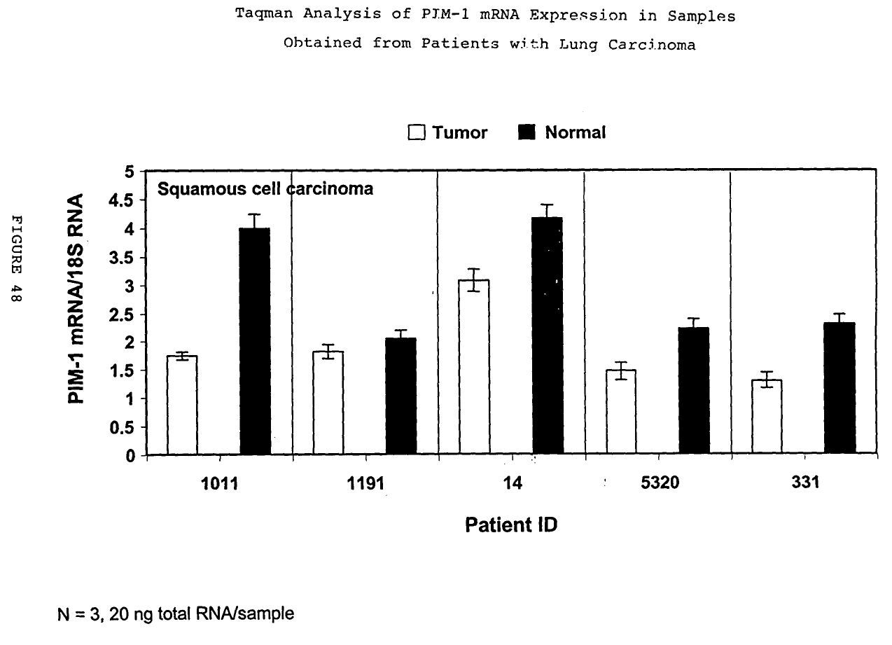

PIM1 ImRNA expression was analyzed in tumor cell lines and primary human tumors. PIM1 mRNA was overexpressed in tumor cell lines (e.g., H1299, PC3, DU145, HCC1937, and MDA-MB-231) as compared to primary cell lines. See, e.g., FIG. 46. PIM1 appeared to be expressed at lower levels in breast carcinomas as compared to normal tissue from the same patient. See, e.g., FIG. 47. PIM1 also appeared to be expressed at lower levels in lung carcinomas as compared to normal tissue from the same patient. See, e.g., FIG. 48.

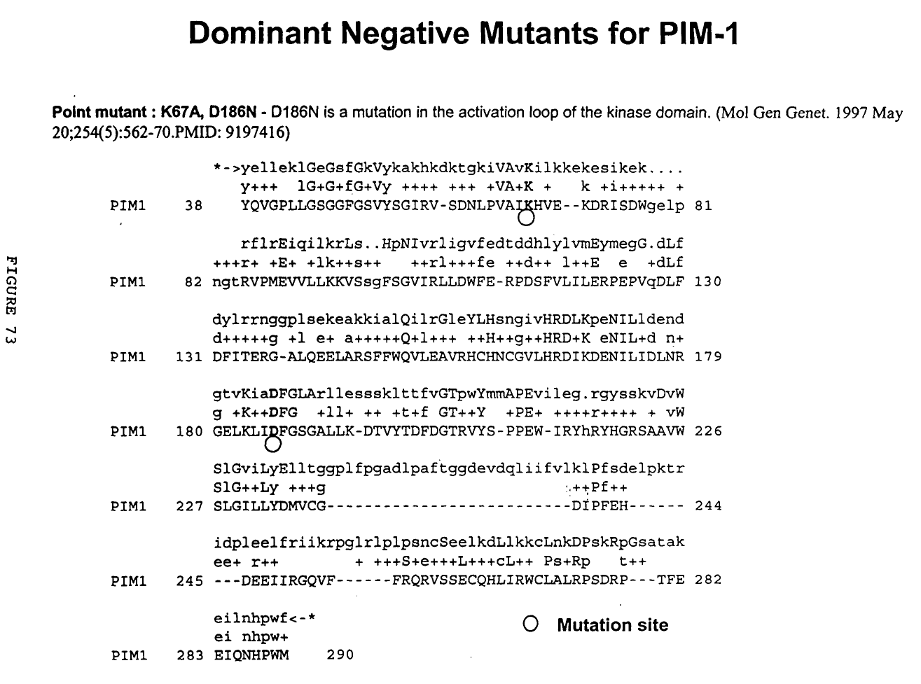

PIM1 mutants were constructed with the following mutations: Pim1 K67A and PIM1 D186N. PIM1 mutants are shown in FIG. 73.

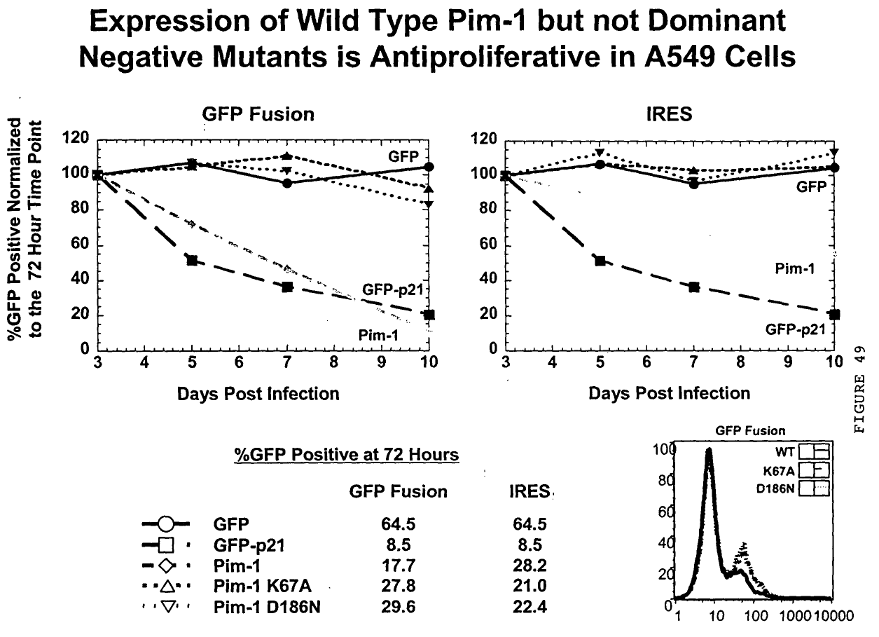

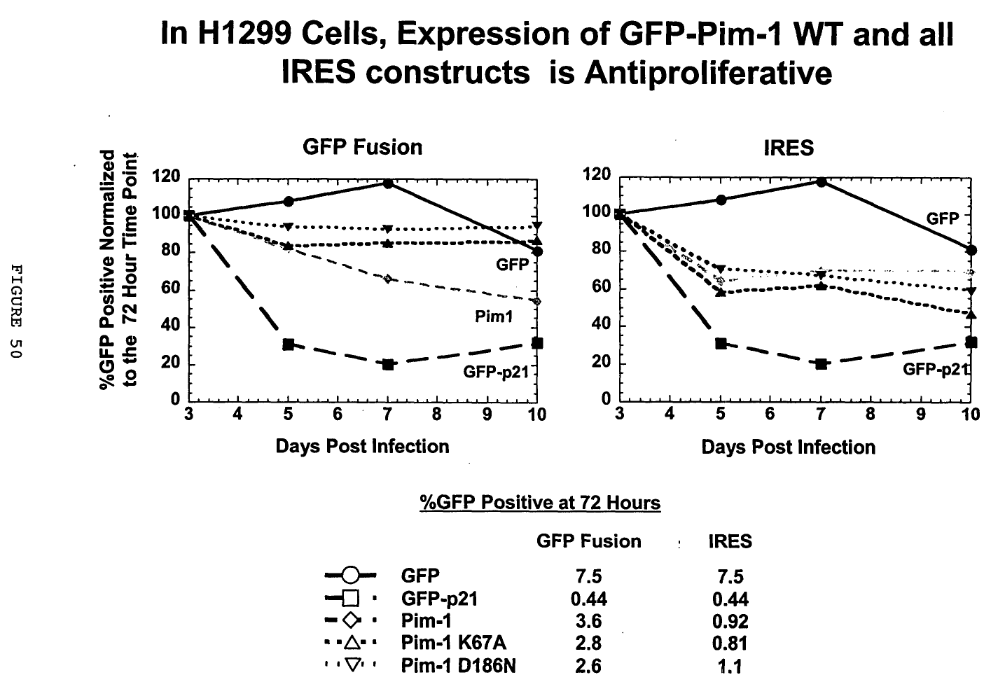

Vectors for the expression of PIM1 fused to the C-terminus of GFP with a tetOff inducible gene expression system were used to transfect A549 cells and H1299 cells. Similar experiments were done using an IRES vector. Cell proliferation was measured using Cell Tracker assays, i.e., detecting GFP positivity. FIG. 49 shows that in A549 cells, expression of wild type PIM1, but not the mutants, was antiproliferative. FIG. 50 shows that in H1299 cells GFP fused wild type PIM1 was antiproliferative. Using IRES constructs, expression of wild type PIM1 and the PIM1 mutants was antiproliferative in H1299 cells.

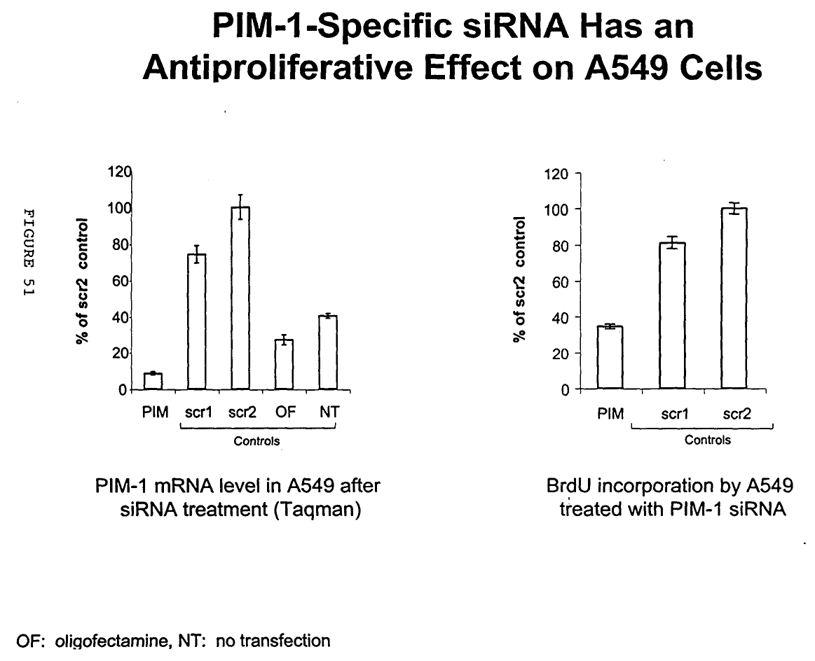

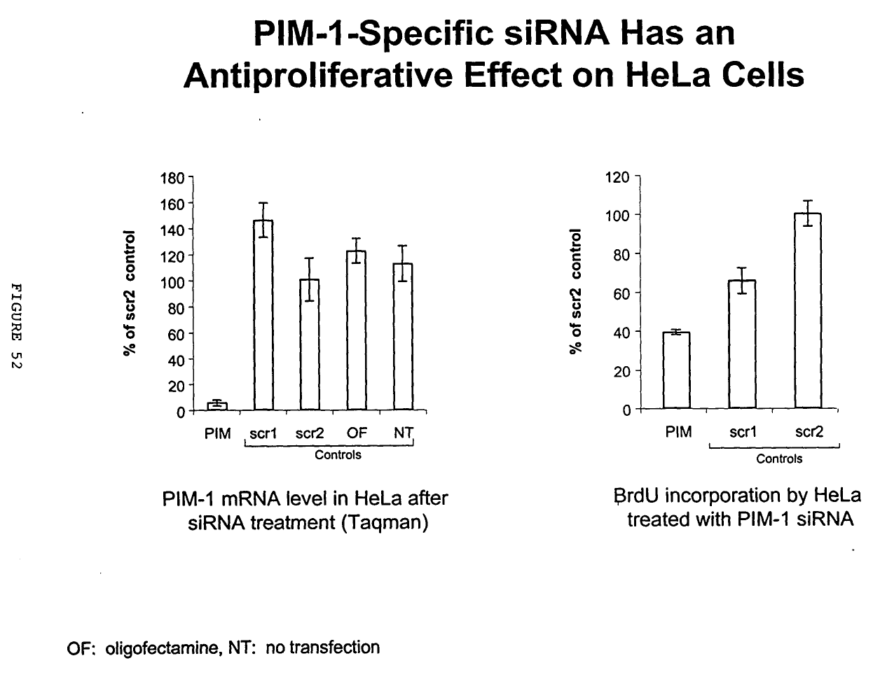

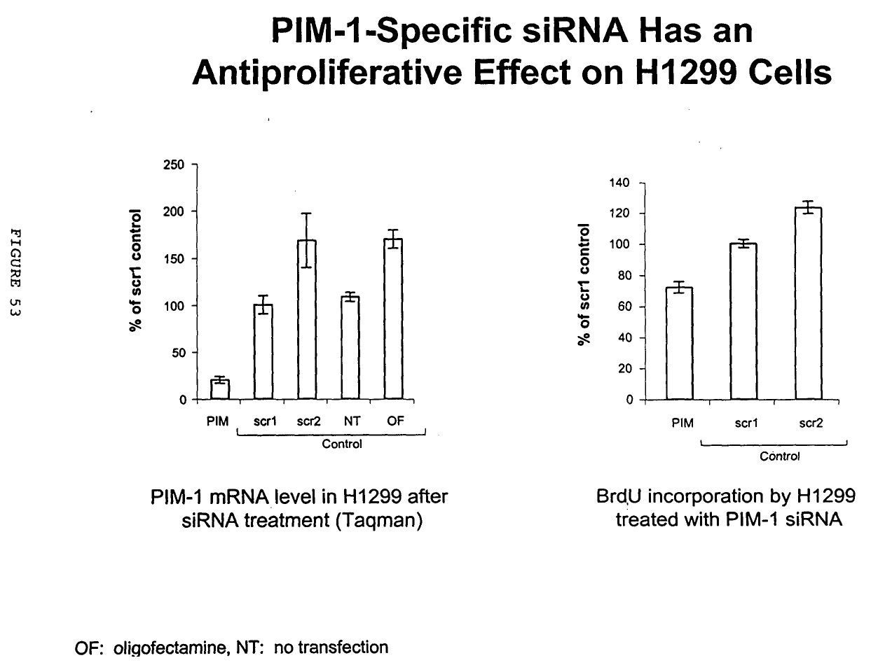

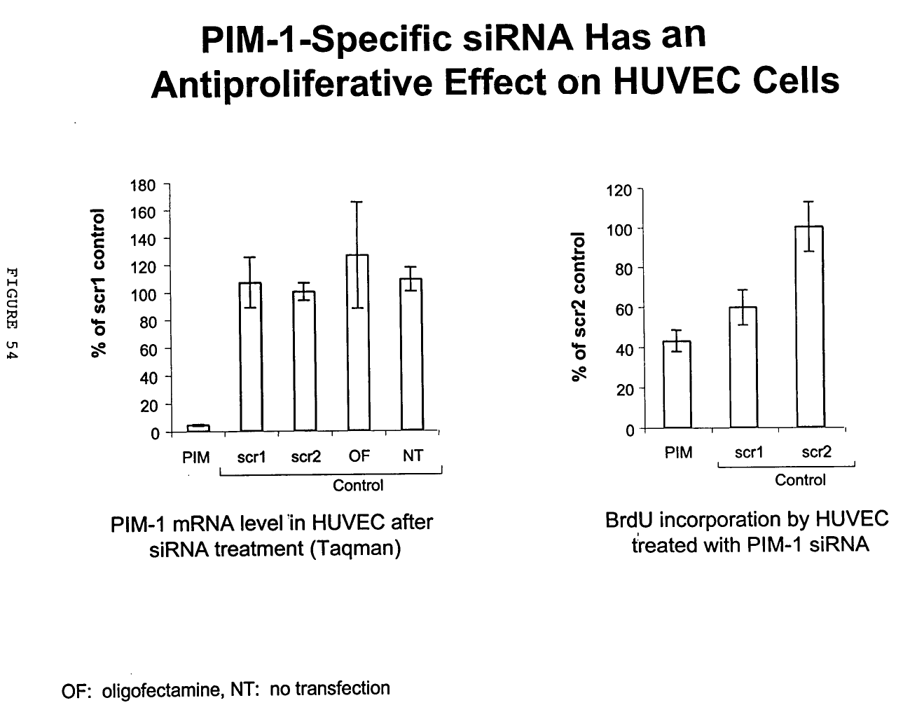

A PIM1-specific siRNA caused greater than 50% reduction in mRNA expression when transfected into A549 cells, HeLa cells, or H1299 cells. The sequence of the PIM1 siRNA is as follows: AAAACTCCGAGTGAACTGGTC. The PIM1 siRNA had an antiproliferative effect when expressed in A549, HeLa cells, or H1299 cells. See, e.g., FIGS. 51-53. In primary HUVEC cells the PIM1-specific siRNA caused greater than 50% reduction in mRNA expression and had an antiproliferative effect. See, e.g., FIG. 54.

Wild type and mutant PIM1 proteins were expressed in Phoenix cells and assayed for kinase activity using Histone H1 as a substrate. Wild type and mutant PIM1 proteins were fused to GFP and also had a myc tag. Wild type and mutant PIM1 proteins were immunoprecipitated using an anti-myc antibody and the immune complexes were assayed for kinase activity using 20 μl of kinase buffer+0.5 μL of γ-³²P ATP (3000 Ci/mmol). Kinase buffer contained 20 mM Tris, pH 7.5; 50 mM NaCl; 10 mM MgCl₂; 2 mM MnCl₂; 1 mM NaF; and 1 mM Na₃VO₄. Kinase reactions were incubated at room temperature for one hour and analyzed by SDS-PAGE and autoradiography. Wild type PIM1 exhibited kinase activity, while the mutant PIM1 proteins did not. (Data not shown.) Western blot analysis was used to show the equivalent amounts of wild type and mutant PIM1 proteins were assayed. (Data not shown.)

### Example 23

### Functional Characterization of APE1

APE1 mutants were constructed with the following mutations: APE1 E96A, APE1 D210A, and APE1 C65A.

Subcellular localization studies demonstrated that APE1 mutant and wild type proteins were localized to the cell nucleus in A549 cells. (Data not shown.)

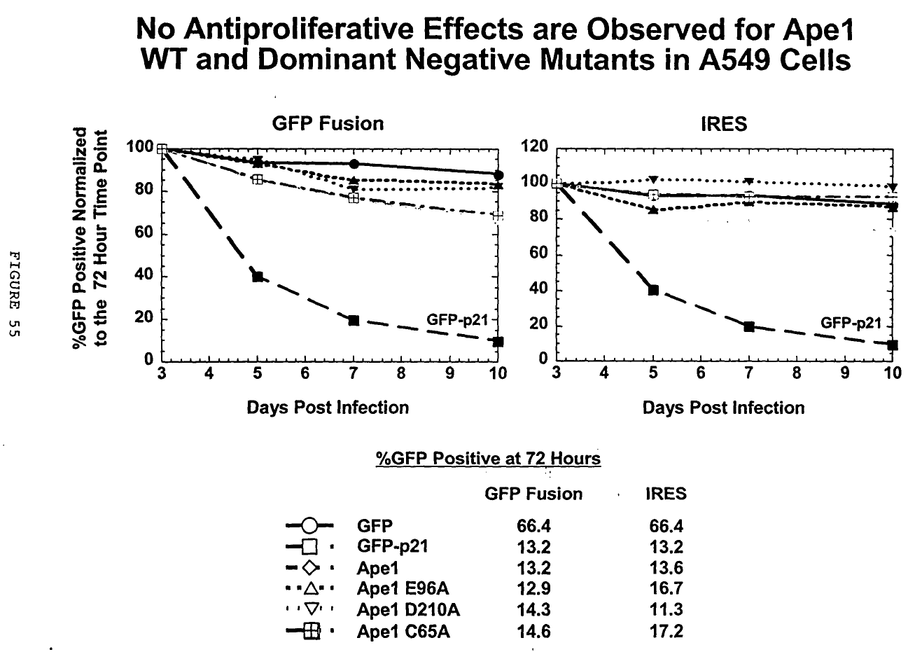

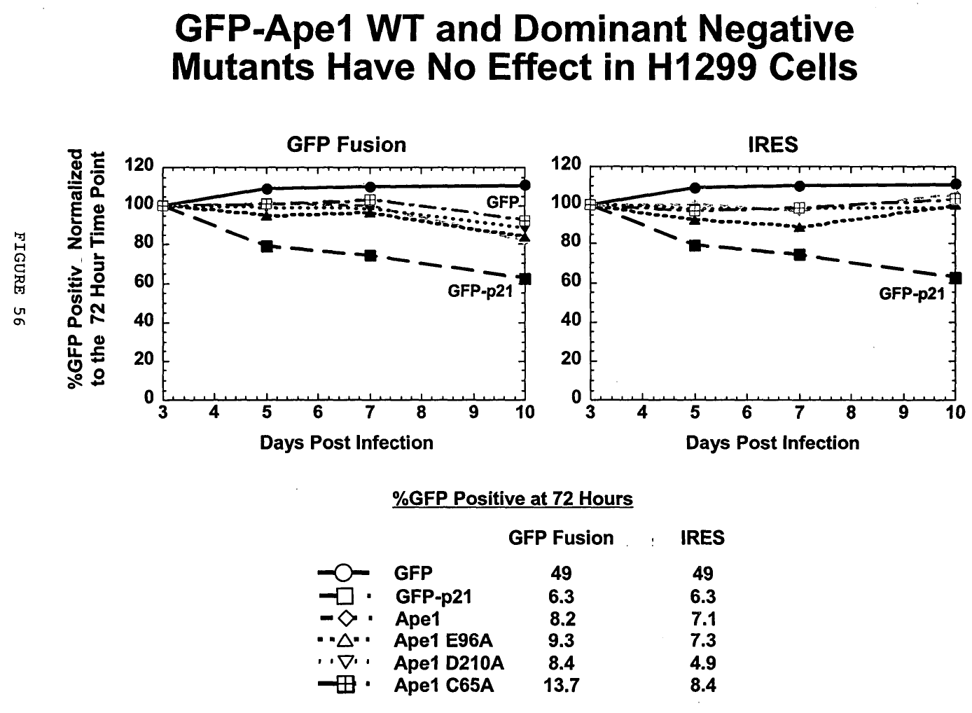

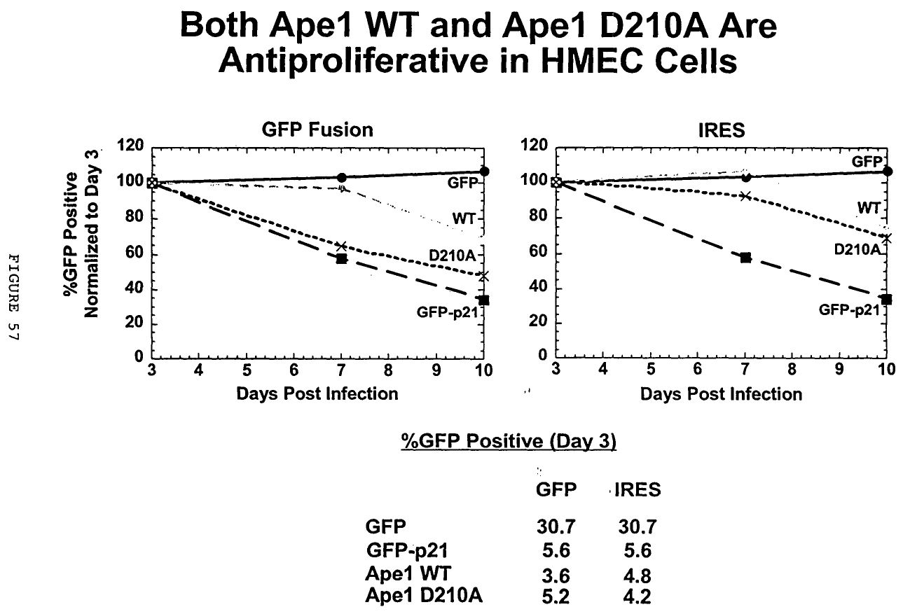

Vectors for the expression of APE1 fused to the C-terminus of GFP with a tetOff inducible gene expression system were used to transfect A549 cells and H1299 cells. APE1 mutants were also expressed. Similar experiments were done using an IRES vector. Cell proliferation was measured using Cell Tracker assays, i.e., detecting GFP positivity. In A549 cells, expression of wild type and mutant APE1 proteins had no apparent effect on proliferation. See, e.g., FIG. 55. Similar results were obtained in H1299 cells. See, e.g., FIG. 56. However, in primary HMEC cells, expression of both wild type APE1 and the APE1 D210A mutant was antiproliferative. See, e.g., FIG. 57.

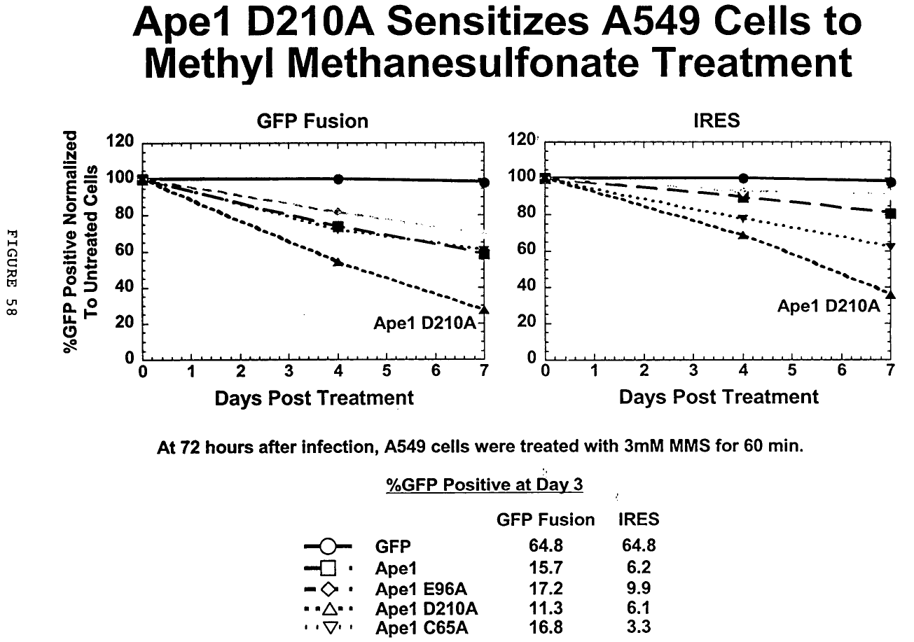

Expression of the APE1 D210A mutant in A549 cells sensitized the cells to methyl methanesulfonante (MMS) treatment. At 72 hours after infection, A549 cells were treated with 3 mM MMS for 60 min. Survival curves are shown in FIG. 58.

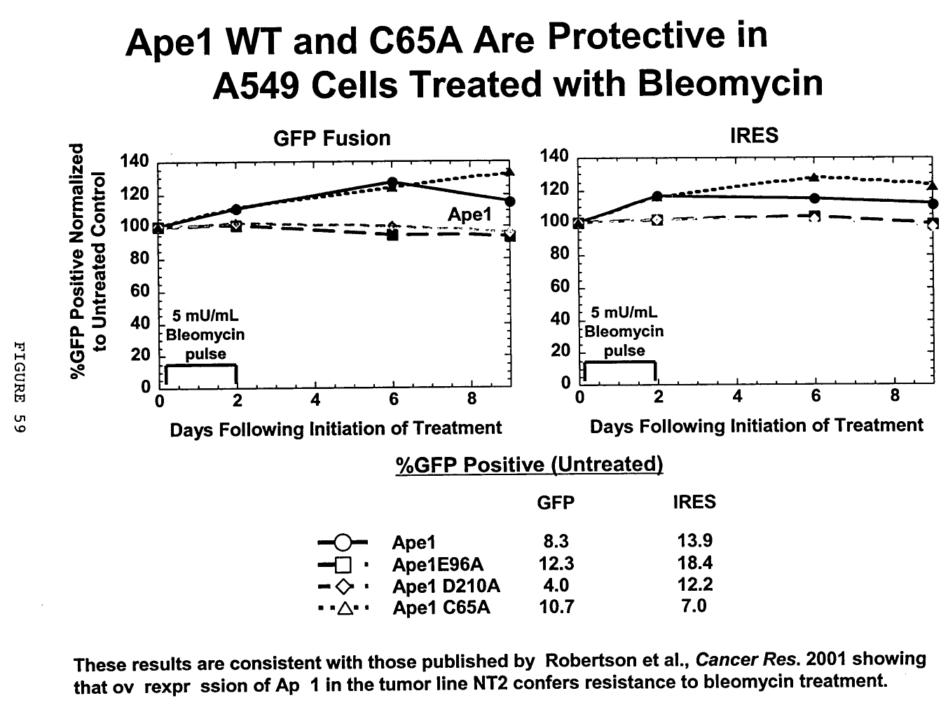

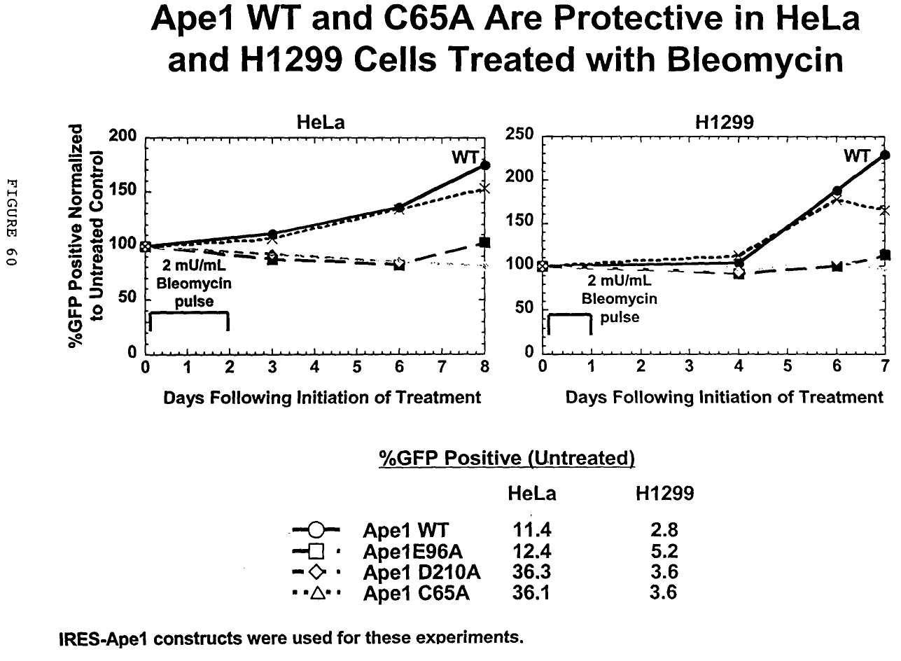

Expression of APE1 wildtype and the APE1 C65A mutant were protective in A549, HeLa, and H1299 cells treated with bleomycin. See, e.g., FIGS. 59-60. These results are consistent with those published by Robertson et al., Cancer Res. 61:2220-5 (2001), showing that overexpression of Ape1 in the tumor line NT2 confers resistance to bleomycin treatment.

### Example 24

### Functional Characterization of Casein kinase II alpha (CK2α or CK2)

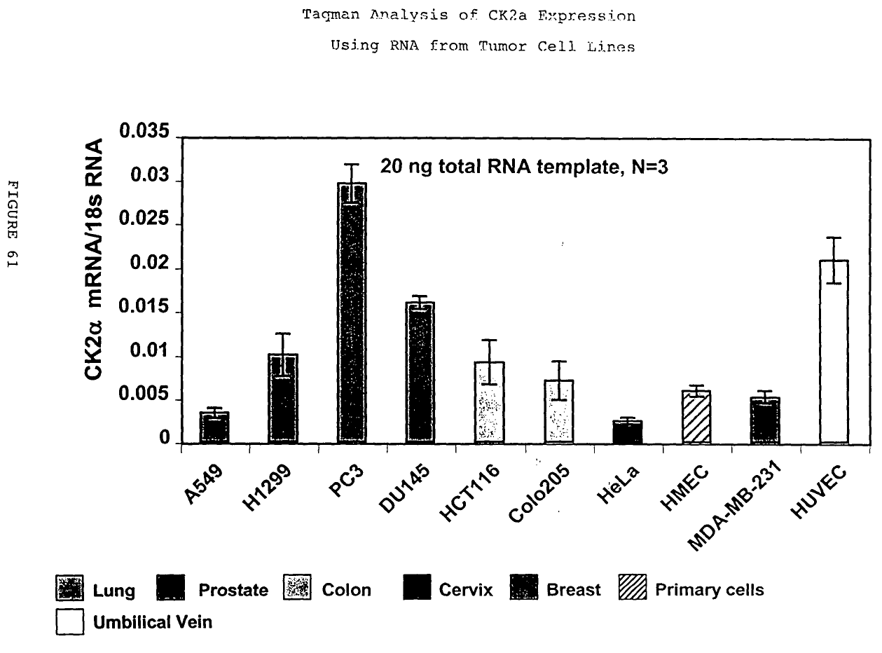

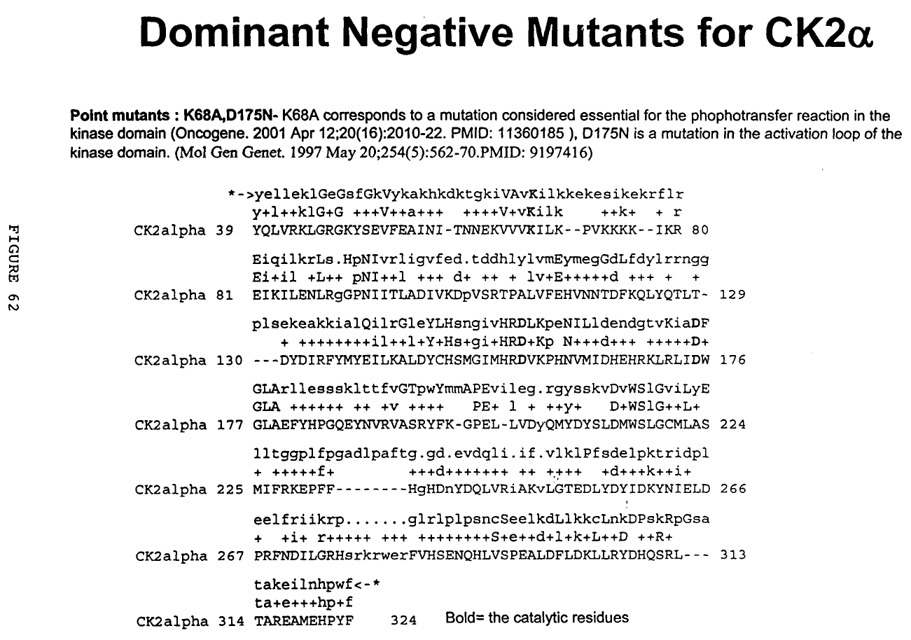

CK2α mRNA expression was analyzed in tumor cell lines and primary human cell lines and results are shown in FIG. 61. CK2α dominant negative mutants are shown in FIG. 62. Subcellular localization studies demonstrated that CK2α mutant and wild type proteins were localized to the cell nucleus and concentrated in punctuate areas outside the nucleus in A549 cells. (Data not shown.) Neither CK2α wild type or mutant protein expression was antiproliferative in A549 or H1299 cells. (Data not shown.)

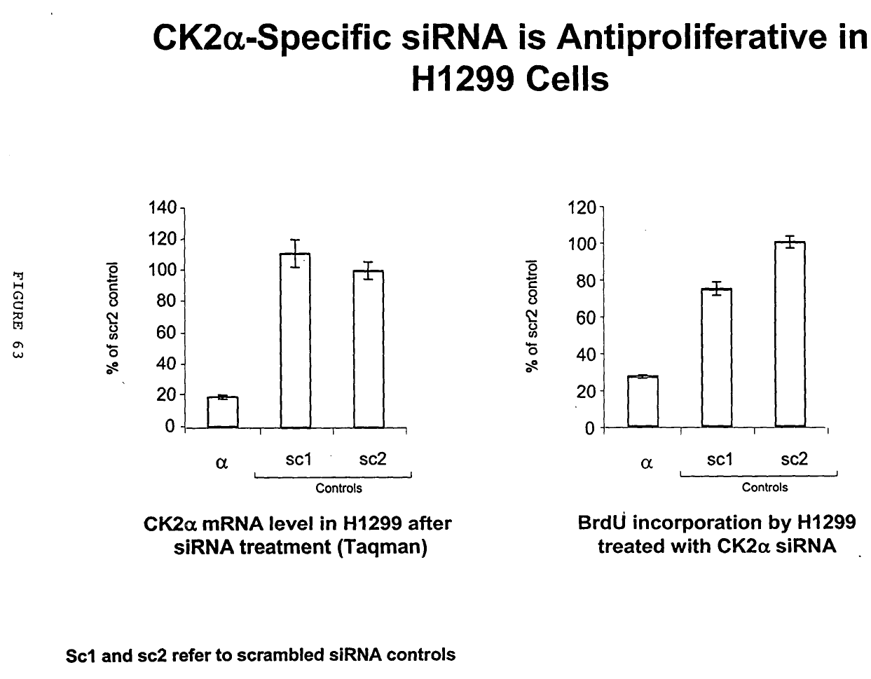

A CK2α-specific siRNA caused greater than 50% reduction in mRNA expression when transfected into H1299 cells. The sequence of the CK2α-specific siRNA (also know as CK2) is as follows: AACATTGAATTAGATCCACGT. The CK2α siRNA had an antiproliferative effect when expressed in H1299 cells. See, e.g., FIG. 63. The same CK2α siRNA reduced mRNA in HeLa cells but did not appear to effect cell proliferation. (Data not shown.)

### Example 25

### Functional Characterization of NKIAMRE

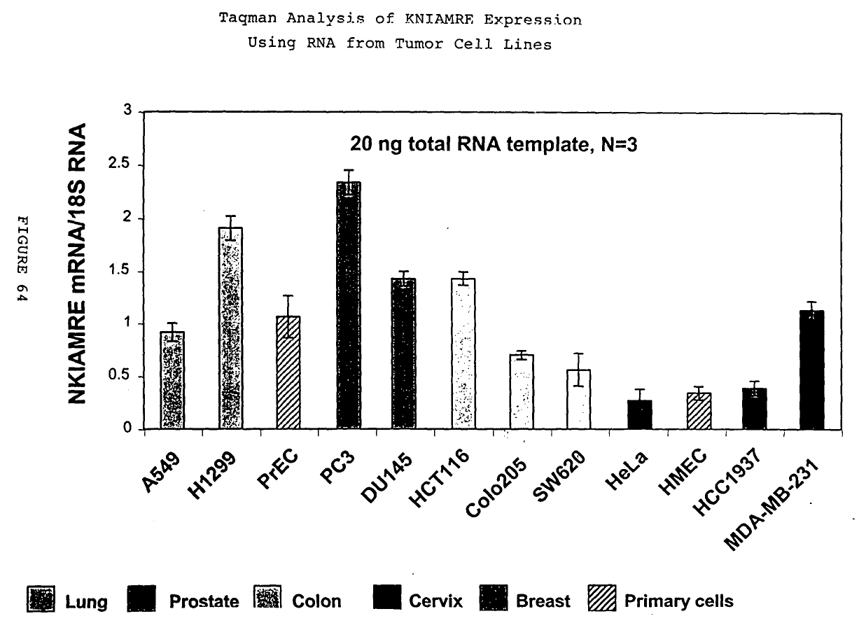

NKIAMRE mRNA expression was analyzed in tumor cell lines. NKIAMRE mRNA was overexpressed in tumor cell lines (e.g., H1299, PC3, DU145, HCT116, and MDA-MB-231) as compared to primary cell lines. See, e.g., FIG. 64. Dominant negative mutants of NKIAMRE were generated and are shown in FIG. 65. Subcellular localization studies demonstrated that NKIAMRE mutant and wild type proteins were localized to the cell cytoplasm in A549 cells. (Data not shown.)

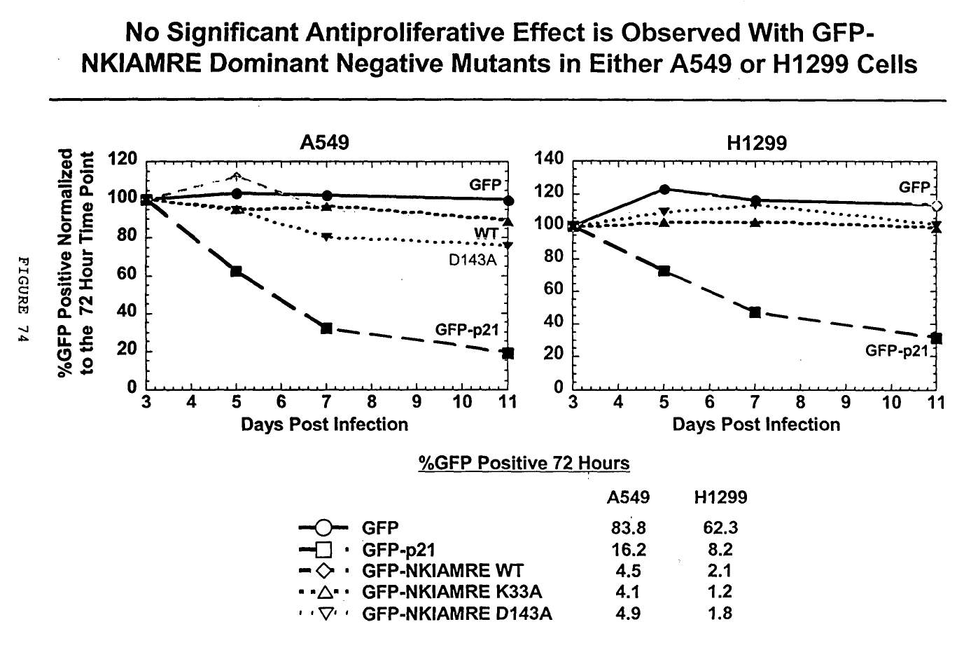

Vectors for the expression of NKIAMRE fused to the C-terminus of GFP with a tetOff inducible gene expression system were used to transfect A549 cells and H1299 cells. NKIAMRE mutants were also expressed. Cell proliferation was measured using Cell Tracker assays, i.e., detecting GFP positivity. In A549 cells and H1299 cells, expression of wild type and mutant NKIAMRE proteins had no apparent effect on proliferation. See, e.g., FIG. 74.

NKIAMRE-specific siRNA caused greater than 50% reduction in mRNA expression when transfected into H1299 cells or HeLa cells, but did not appear to affect proliferation in either cell line. Data not shown.

### Example 26

### Functional Characterization of FEN1

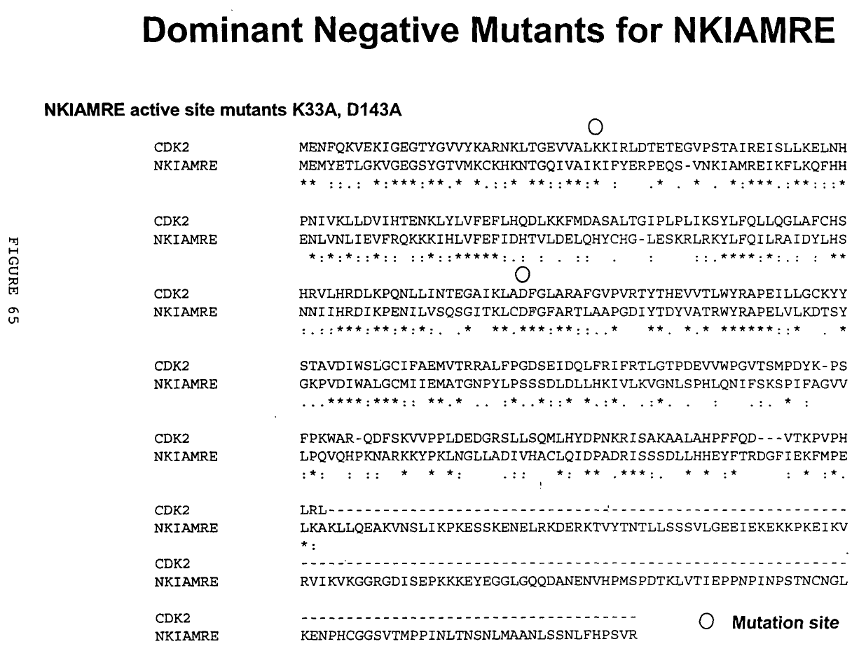

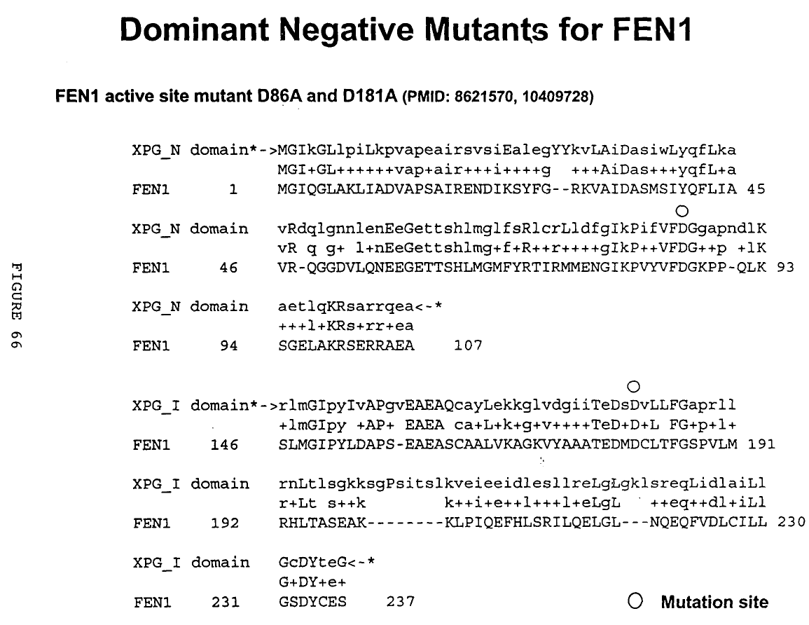

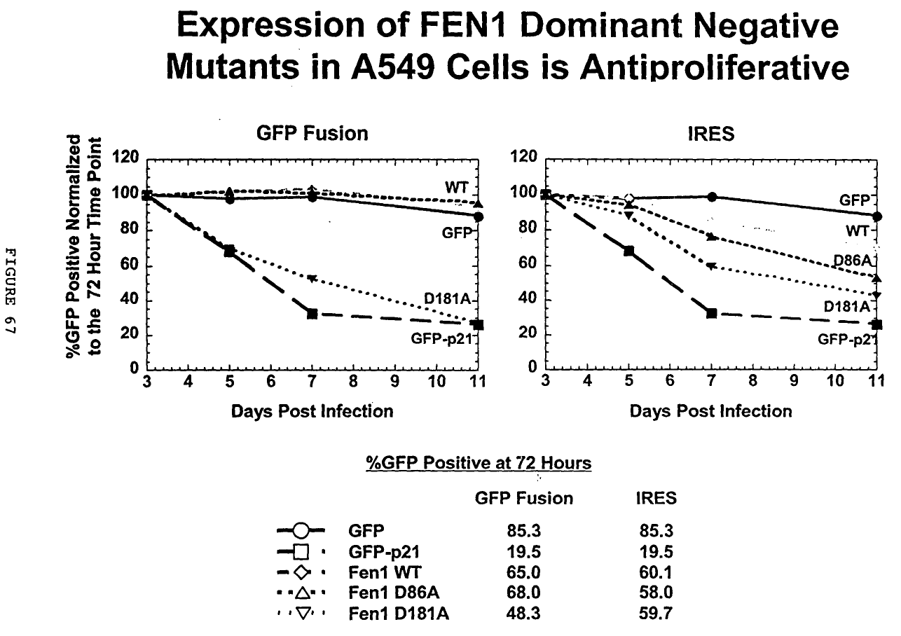

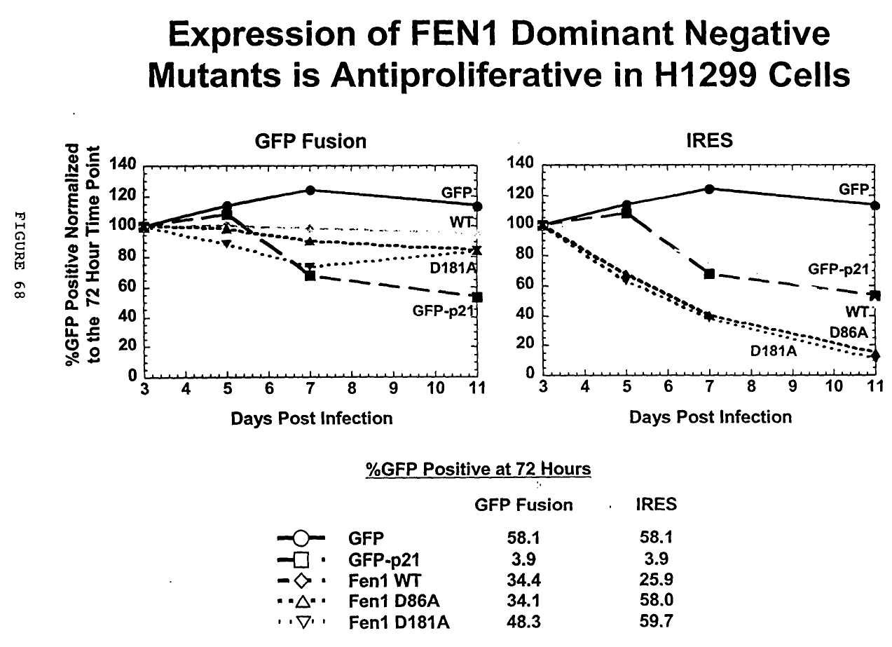

Dominant negative mutants of FEN1 were generated and are shown in FIG. 66. Vectors for the expression of FEN1 fused to the C-terminus of GFP with a tetOff inducible gene expression system were used to transfect A549 cells and H1299 cells. GFP fusions were also made using the FEN1 dominant negative mutants. Similar experiments were done using an IRES vector. Cell proliferation was measured using Cell Tracker assays, i.e., detecting GFP positivity. FIG. 67 shows that in A549 cells, expression of mutant FEN1, but not the wild type, was antiproliferative. FIG. 68 shows that in H1299 cells, expression of the FEN1 dominant negative mutants was also antiproliferative.

### Example 27

### Functional Characterization of CDK3

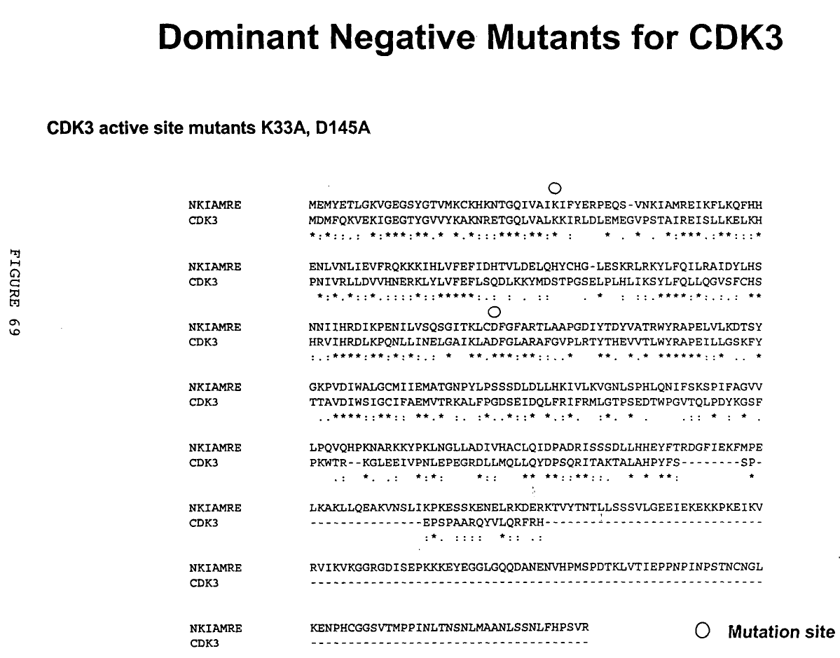

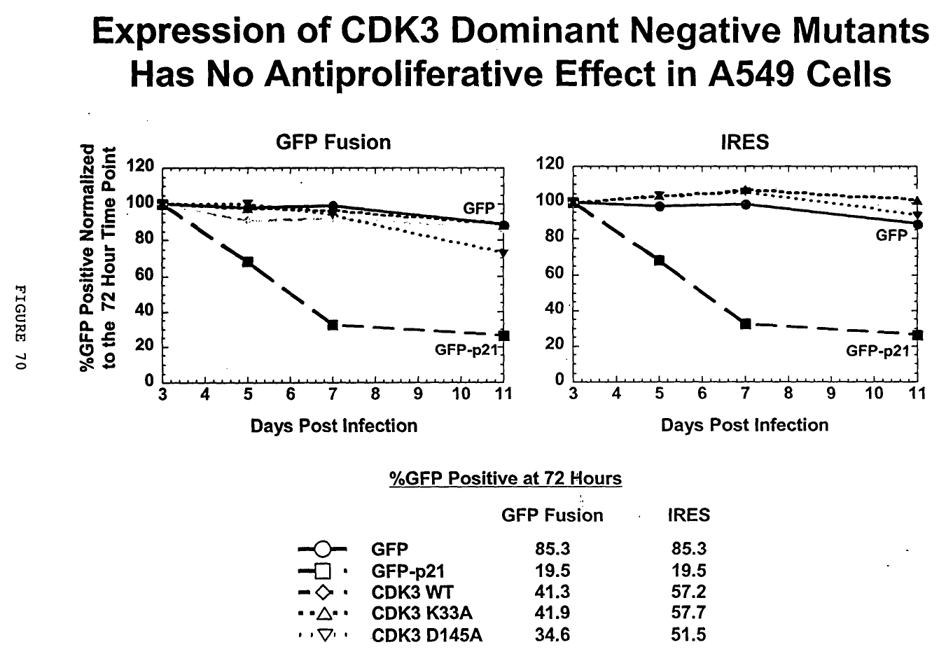

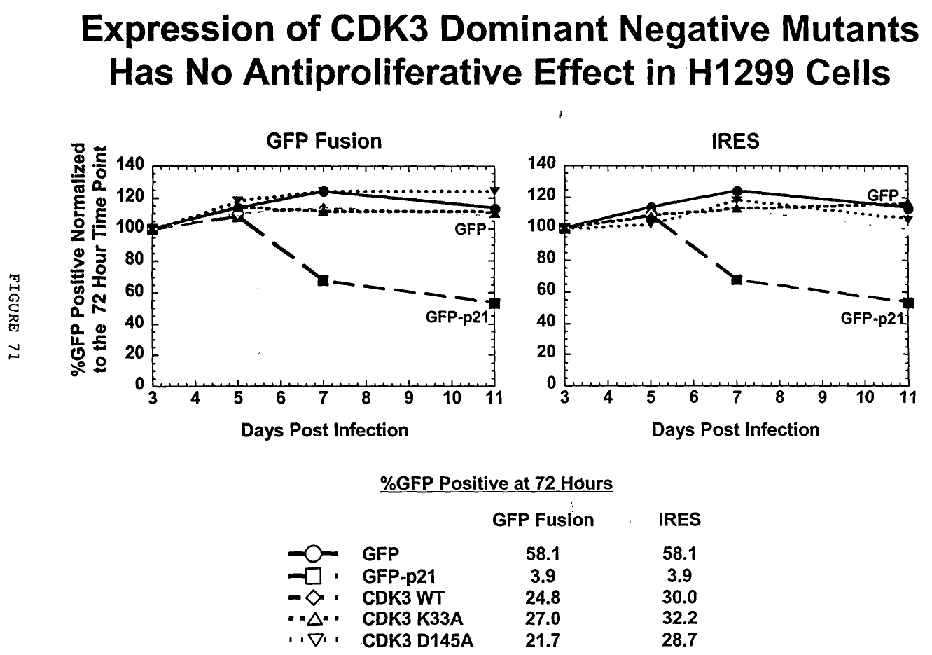

Dominant negative mutants of CDK3 were generated and are shown in FIG. 69. Vectors for the expression of CDK3 fused to the C-terminus of GFP with a tetOff inducible gene expression system were used to transfect A549 cells and H1299 cells. GFP fusions were also made using the CDK3 dominant negative mutants. Similar experiments were done using an IRES vector. Cell proliferation was measured using Cell Tracker assays, i.e., detecting GFP positivity. FIG. 70 shows that in A549 cells, expression of either wild type CDK3 or mutant CDK3 proteins had no apparent antiproliferative effect. FIG. 71 shows that in H1299 cells, expression of either wild type CDK3 or mutant CDK3 proteins had no apparent antiproliferative effect.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for identifying a compound that modulates cell cycle arrest, the method comprising the steps of:
(i) contacting a cell comprising a target polypeptide or fragment thereof or inactive variant thereof, selected from the group consisting of flap structure specific endonuclease 1 (FEN1), protein kinase C ζ (PKC-ζ), phospholipase C-β1 (PLC-β1), protein tyrosine kinase 2 (FAK), protein tyrosine kinase 2b (FAK2), casein kinase 2 (CK2), cMET tyrosine kinase (cMET), REV1 dCMP transferase (REV 1), apurinic/apyrimidinic nuclease 1 (APE1), cyclin dependent kinase 3 (CDK3), PIM1 kinase (PIM1), cell division cycle 7 kinase (CDC7L1), cyclin dependent kinase 7 (CDK7), cytokine inducible kinase (CNK), potentially prenylated protein tyrosine phosphatase (PRL-3), serine threonine kinase 2 (STK2) or (NEK4), cyclin dependent serine threonine kinase (NKIAMRE), or histone acetylase (HBO1), or fragment thereof with the compound, the target polypeptide encoded by the complement of a nucleic acid that hybridizes under stringent conditions to a nucleic acid encoding a polypeptide having an amino acid sequence selected from the group consisting of SEQ ID NO:14, 2, 4, 6, 8, 10, 12, 16, 18, 20, 22, 24, 26, 28, 30, 32, 34, and 36; and
(ii) determining the chemical or phenotypic effect of the compound upon the cell comprising the target polypeptide or fragment thereof or inactive variant thereof, thereby identifying a compound that modulates cell cycle arrest.

2. The method of claim 1, wherein the chemical or phenotypic effect is determined by measuring enzymatic activity selected from the group consisting of nuclease activity, kinase activity, lipase activity, transferase activity, phosphatase activity, and acetylase activity.

3. The method of claim 1, wherein the chemical or phenotypic effect is determined by measuring cellular proliferation.

4. The method of claim 3, wherein the cellular proliferation is measured by assaying fluorescent marker level or DNA synthesis.

5. The method of claim 4, wherein DNA synthesis is measured by ³H thymidine incorporation, BrdU incorporation, or Hoescht staining.

6. The method of claim 4, wherein the fluorescent marker is selected from the group consisting of a cell tracker dye or green fluorescent protein.

7. The method of claim 1, wherein modulation is activation of cell cycle arrest.

8. The method of claim 1, wherein modulation is activation of cancer cell cycle arrest.

9. The method of claim 1, wherein the host cell is a cancer cell.

10. The method of claim 9, wherein the cancer cell is a breast, prostate, colon, or lung cancer cell.

11. The method of claim 9, wherein the cancer cell is a transformed cell line.

12. The method of claim 11, wherein the transformed cell line is A549, PC3, H1299, MDA-MB-231, MCF7, or HeLa.

13. The method of claim 9, wherein the cancer cell is p53 null or mutant.

14. The method of claim 9, wherein the cancer cell is p53 wild-type.

15. The method of claim 1, wherein the polypeptide is recombinant.

16. The method of claim 1, wherein the polypeptide is encoded by a nucleic acid comprising a sequence of SEQ ID NO:13, 1, 3, 5, 7, 9, 11, 15, 17, 19, 21, 23, 25, 27, 29, 31, 33, or 35.

17. The method of claim 1, wherein the compound is an antibody.

18. The method of claim 1, wherein the compound is a small organic molecule.

19. The method of claim 1, wherein the compound is an antisense molecule.

20. The method of claim 1, wherein the compound is a peptide.

21. The method of claim 20, wherein the peptide is circular.

22. The method of claim 1, wherein the compound is an siRNA molecule.

23. A method for identifying a compound that modulates cell cycle arrest, the method comprising the steps of:
(i) contacting a cell comprising a target polypeptide or fragment thereof or inactive variant thereof, selected from the group consisting of flap structure specific endonuclease 1 (FEN1), protein kinase C ζ (PKC-ζ), phospholipase C-β1 (PLC-β1), protein tyrosine kinase 2 (FAK), protein tyrosine kinase 2b (FAK2), casein kinase 2 (CK2), cMET tyrosine kinase (cMET), REV1 dCMP transferase (REV 1), apurinic/apyrimidinic nuclease 1 (APE1), cyclin dependent kinase 3 (CDK3), PIM1 kinase (PIM1), cell division cycle 7 kinase (CDC7L1), cyclin dependent kinase 7 (CDK7), cytokine inducible kinase (CNK), potentially prenylated protein tyrosine phosphatase (PRL-3), serine threonine kinase 2 (STK2) or (NEK4), cyclin dependent serine threonine kinase (NKIAMRE), or histone acetylase (HBO1), or fragment thereof with the compound, the target polypeptide encoded by the complement of a nucleic acid that hybridizes under stringent conditions to a nucleic acid encoding a polypeptide having an amino acid sequence selected from the group consisting of SEQ ID NO:14, 2, 4, 6, 8, 10, 12, 16, 18, 20, 22, 24, 26, 28, 30, 32, 34, and 36; and
(ii) determining the physical effect of the compound upon the target polypeptide or fragment thereof or inactive variant thereof; and
(iii) determining the chemical or phenotypic effect of the compound upon a cell comprising the target polypeptide or or fragment thereof or inactive variant thereof, thereby identifying a compound that modulates cell cycle arrest.

24. A method of modulating cell cycle arrest in a subject, the method comprising the step of administering to the subject a therapeutically effective amount of a compound identified using the method of claim 1.

25. The method of claim 24, wherein the subject is a human.

26. The method of claim 25, wherein the subject has cancer.

27. The method of claim 24, wherein the compound is a small organic molecule.

28. The method of claim 24, wherein the compound is an antisense molecule.

29. The method of claim 24, wherein the compound is an antibody.

30. The method of claim 24, wherein the compound is a peptide.

31. The method of claim 30, wherein the peptide is circular.

32. The method of claim 24, wherein the compound is an siRNA molecule.

33. The method of claim 24, wherein the compound inhibits cancer cell proliferation.

34. A method of modulating cell cycle arrests in a subject, the method comprising the step of administering to the subject a therapeutically effective amount of a target polypeptide or fragment thereof or inactive variant thereof, selected from the group consisting of flap structure specific endonuclease 1 (FEN1), protein kinase C ζ (PKC-ζ), phospholipase C-β1 (PLC-β1), protein tyrosine kinase 2 (FAK), protein tyrosine kinase 2b (FAK2), casein kinase 2 (CK2), cMET tyrosine kinase (cMET), REV1 dCMP transferase (REV 1), apurinic/apyrimidinic nuclease 1 (APE1), cyclin dependent kinase 3 (CDK3), PIM1 kinase (PIM1), cell division cycle 7 kinase (CDC7L1), cyclin dependent kinase 7 (CDK7), cytokine inducible kinase (CNK), potentially prenylated protein tyrosine phosphatase (PRL-3), serine threonine kinase 2 (STK2) or (NEK4), cyclin dependent serine threonine kinase (NKIAMRE), or histone acetylase (HBO1), or fragment thereof with the compound, the target polypeptide encoded by the complement of a nucleic acid that hybridizes under stringent conditions to a nucleic acid encoding a polypeptide having an amino acid sequence selected from the group consisting of SEQ ID NO:14, 2, 4, 6, 8, 10, 12, 16, 18, 20, 22, 24, 26, 28, 30, 32, 34, and 36.

35. A method of modulating cell cycle arrest in a subject, the method comprising the step of administering to the subject a therapeutically effective amount of a nucleic acid encoding a target polypeptide or fragment thereof or inactive variant thereof, selected from the group consisting of flap structure specific endonuclease 1 (FEN1), protein kinase C ζ(PKC-ζ), phospholipase C-β1 (PLC-β1), protein tyrosine kinase 2 (FAK), protein tyrosine kinase 2b (FAK2), casein kinase 2 (CK2), cMET tyrosine kinase (cMET), REV1 dCMP transferase (REV1), apurinic/apyrimidinic nuclease 1 (APE1), cyclin dependent kinase 3 (CDK3), PIM1 kinase (PIM1), cell division cycle 7 kinase (CDC7L1), cyclin dependent kinase 7 (CDK7), cytokine inducible kinase (CNK), potentially prenylated protein tyrosine phosphatase (PRL-3), serine threonine kinase 2 (STK2) or (NEK4), cyclin dependent serine threonine kinase (NKIAMRE), or histone acetylase (HBO1), or fragment thereof with the compound, the target polypeptide encoded by the complement of a nucleic acid that hybridizes under stringent conditions to a nucleic acid encoding a polypeptide having an amino acid sequence selected from the group consisting of SEQ ID NO:14, 2, 4, 6, 8, 10, 12, 16, 18, 20, 22, 24, 26, 28, 30, 32, 34, and 36.

36. A CK2-specific siRNA molecule comprising the sequence AACATTGAATTAGATCCACGT, wherein the siRNA molecule is from 21 to 30 nucleotide base pairs in length.

37. The CK2-specific siRNA molecule of claim 36 consisting of the sequence AACATTGAATTAGATCCACGT and its complement as active portion.

38. A method of inhibiting expression of a CK2 gene in a cell, the method comprising contacting the cell with a CK2-specific siRNA molecule comprising the sequence AACATTGAATTAGATCCACGT, wherein the siRNA molecule is from 21 to 30 nucleotide base pairs in length.

39. A PIM1-specific siRNA molecule comprising the sequence AAAACTCCGAGTGAACTGGTC, wherein the siRNA molecule is from 21 to 30 nucleotide base pairs in length.

40. The PIM1-specific siRNA molecule of claim 39 consisting of the sequence AAAACTCCGAGTGAACTGGTC and its complement as active portion.

41. A method of inhibiting expression of a PIM1 gene in a cell, the method comprising contacting the cell with a PIM1-specific siRNA molecule comprising the sequence AAAACTCCGAGTGAACTGGTC, wherein the siRNA molecule is from 21 to 30 nucleotide base pairs in length.

42. An Hbo1-specific siRNA molecule comprising the sequence AACTGAGCAAGTGGTTGATTT, wherein the siRNA molecule is from 21 to 30 nucleotide base pairs in length.

43. The Hbo 1-specific siRNA molecule of claim 42 consisting of the sequence AACTGAGCAAGTGGTTGATTT and its complement as active portion.

44. A method of inhibiting expression of an Hbo 1 gene in a cell, the method comprising contacting the cell with an Hbo1-specific siRNA molecule comprising the sequence AACTGAGCAAGTGGTTGATTT, wherein the siRNA molecule is from 21 to 30 nucleotide base pairs in length.


SEQUENCE LISTING (78 sequences)

SEQ ID NO: 1 - DNA, 2164 bp, Homo sapiens
  protein kinase C, zeta (PKC-zeta), atypical protein kinase C isoform
atgcccagcaggaccgaccccaagatggaagggagcggcggccgcgtccgcctcaaggcgcattacgggggggacatcttcatcaccagcgtggacgccgccacgaccttcgaggagctctgtgaggaagtgagagacatgtgtcgtctgcaccagcagcacccgctcaccctcaagtgggtggacagcgaaggtgacccttgcacggtgtcctcccagatggagctggaagaggctttccgcctggcccgtcagtgcagggatgaaggcctcatcattcatgttttcccgagcacccctgagcagcctggcctgccatgtccgggagaagacaaatctatctaccgccggggagccagaagatggaggaagctgtaccgtgccaacggccacctcttccaagccaagcgctttaacaggagagcgtactgcggtcagtgcagcgagaggatatggggcctcgcgaggcaaggctacaggtgcatcaactgcaaactgctggtccataagcgctgccacggcctcgtcccgctgacctgcaggaagcatatggattctgtcatgccttcccaagagcctccagtagacgacaagaacgaggacgccgaccttccttccgaggagacagatggaattgcttacatttcctcatcccggaagcatgacagcattaaagacgactcggaggaccttaagccagttatcgatgggatggatggaatcaaaatctctcaggggcttgggctgcaggactttgacctaatcagagtcatcgggcgcgggagctacgccaaggttctcctggtgcggttgaagaagaatgaccaaatttacgccatgaaagtggtgaagaaagagctggtgcatgatgacgaggatattgactgggtacagacagagaagcacgtgtttgagcaggcatccagcaaccccttcctggtcggattacactcctgcttccagacgacaagtcggttgttcctggtcattgagtacgtcaacggcggggacctgatgttccacatgcagaggcagaggaagctccctgaggagcacgccaggttctacgcggccgagatctgcatcgccctcaacttcctgcacgagagggggatcatctacagggacctgaagctggacaacgtcctcctggatgcggacgggcacatcaagctcacagactacggcatgtgcaaggaaggcctgggccctggtgacacaacgagcactttctgcggaaccccgaattacatcgcccccgaaatcctgcggggagaggagtacgggttcagcgtggactggtgggcgctgggagtcctcatgtttgagatgatggccgggcgctccccgttcgacatcatcaccgacaacccggacatgaacacagaggactaccttttccaagtgatcctggagaagcccatccggatcccccggttcctgtccgtcaaagcctcccatgttttaaaaggatttttaaataaggaccccaaagagaggctcggctgccggccacagactggattttctgacatcaagtcccacgcgttcttccgcagcatagactgggacttgctggagaagaagcaggcgctccctccattccagccacagatcacagacgactacggtctggacaactttgacacacagttcaccagcgagcccgtgcagctgaccccagacgatgaggatgccataaagaggatcgaccagtcagagttcgaaggctttgagtatatcaacccattattgctgtccaccgaggagtcggtgtgaggccgcgtgcgtctctgtcgtggacacgcgtgattgaccctttaactgtatccttaaccaccgcatatgcatgccaggctgggcacggctccgagggcggccagggacagacgcttgcgccgagaccgcagagggaagcgtcagcgggcgctgctgggagcagaacagtccctcacacctggcccggcaggcagcttcgtgctggaggaacttgctgctgtgcctgcgtcgcggcggatccgcggggaccctgccgagggggctgtcatgcggtttccaaggtgcacattttccacggaaacagaactcgatgcactgacctgctccgccaggaaagtgagcgtgtagcgtcctgaggaataaaatgttccgatgaaaaaaaaaa

SEQ ID NO: 2 - protein, 592 aa, Homo sapiens [sequence omitted]

SEQ ID NO: 3 - DNA, 3663 bp, Homo sapiens
  phosphoinositide-specific phospholipase C beta 1, isoform a (PLC-beta1), transcript variant 1
cagatggccggggctcaacccggagtgcacgccttgcaactcaagcccgtgtgcgtgtccgacagcctcaagaagggcaccaaattcgtcaagtgggatgatgattcaactattgttactccaattattttgaggactgaccctcagggatttttcttttactggacagatcaaaacaaggagacagagctactggatctcagccttgtcaaagatgccagatgtgggagacacgccaaagctcccaaggaccccaaattacgtgaacttttggatgtggggaacatcgggcgcctggagcagcgcatgatcacagtggtgtatgggcctgacctcgtgaacatctcccatttgaatctcgtggctttccaagaagaagtggccaaggaatggacaaatgaggttttcagtttggcaacaaacctgctggcccaaaacatgtccagggatgcatttctggaaaaagcctatactaaacttaagctgcaagtcactccagaagggcgtattcctctcaaaaacatatatcgcttgttttcagcagatcggaagcgagttgaaactgctttagaggcttgtagtcttccatcttcaaggaatgattcaatacctcaagaagatttcactccagaagtgtacagagttttcctcaacaacctttgccctcgacctgaaattgataacatcttttcagaatttggtgcaaaaagcaaaccatatcttaccgttgatcagatgatggattttatcaaccttaagcagcgagatcctcggcttaatgaaatactttatccacctctaaaacaagagcaagtccaagtattgattgagaagtatgaacccaacaacagcctcgccagaaaaggacaaatatcagtggatgggttcatgcgctatctgagtggagaagaaaacggagtcgtttcacctgagaaactggatttgaatgaagacatgtctcagcccctttctcactatttcattaattcctcgcacaacacctacctcacagctggccaactggctggaaactcctctgttgagatgtatcgccaagtgctcctgtctggttgtcgctgtgtggagctggactgctggaagggacggactgcagaagaggaacctgtcatcacccatggcttcaccatgacaactgaaatatctttcaaggaagtgatagaagcaattgcggagtgtgcatttaagacttcaccttttccaattctcctttcgtttgagaaccatgtggattccccaaagcagcaagccaagatggcggagtactgccgactgatctttggggatgcccttctcatggagcccctggaaaaatatccactggaatctggagttcctcttccaagccctatggatttaatgtataaaattttggtgaaaaataagaagaaatcacacaagtcatcagaaggaagcggcaaaaagaagctctcagaacaagcctccaacacctacagtgactcctccagcatgttcgagccctcatccccaggagccggagaagctgatacggaaagtgacgacgacgatgatgatgatgactgtaaaaaatcttcaatggatgaggggactgctggaagtgaggctatggccacagaagaaatgtctaatctggtgaactatattcagccagtcaagtttgagtcatttgaaatttcaaaaaaaagaaataaaagttttgaaatgtcttccttcgtggaaaccaaaggacttgaacaactcaccaagtctccagtggaatttgtagaatataacaaaatgcagcttagcaggatatatccaaaaggaacacgtgtggattcatccaactatatgcctcagctcttctggaatgcaggttgtcagatggtggcacttaatttccagacaatggacctggctatgcaaataaatatggggatgtatgaatacaacgggaagagtggctacagattgaagccagagttcatgaggaggcctgacaagcattttgatccatttactgaaggcatcgtagatgggatagtggcaaacactttgtctgttaagattatttcaggtcagtttctttctgataagaaagttgggacttacgtggaagtagatatgtttggtttgcctgtggatacaaggaggaaggcatttaagaccaaaacatcccaaggaaatgctgtgaatcctgtctgggaagaagaacctattgtgttcaaaaaggtggttcttcctactctggcctgtttgagaatagcagtttatgaagaaggaggtaaattcattggccaccgtatcttgccagtgcaagccattcggccaggctatcactatatctgtctaaggaatgaaaggaaccagcctctgacgctgcctgctgtctttgtctacatagaagtgaaagactatgtgccagacacatatgcagatgtcatcgaagctttatcaaacccaatccgatatgtgaacctgatggaacagagagctaagcaattggctgctttgacactggaagatgaagaagaagtaaagaaagaggctgatcctggagaaacaccatcagaggctccaagtgaagcgagaacgactccagcagaaaatggggtgaatcacactacaaccctgacacccaagccaccctcccaggctctccacagccagccagctccaggttctgtaaaggcacctgccaaaacagaagatcttattcagagtgtcttaacagaagtggaagcacagaccatcgaagaactaaagcaacagaaatcgtttgtgaaacttcaaaagaaacactacaaagaaatgaaagacctggttaagagacaccacaagaaaaccactgaccttatcaaagaacacactaccaagtataatgaaattcagaatgactacttgagaaggagagccgctttggaaaagtccgccaaaaaggacagtaagaaaaaatcggaacccagcagccctgatcatggttcatcaacgattgagcaagacctcgctgctctggatgctgaaatgacccaaaagttaatagacttgaaggacaaacaacagcagcagctgcttaatcttcggcaagaacagtattatagtgaaaaataccagaagcgagaacatattaaactgcttattcaaaagttgacggatgtcgcagaagagtgtcagaacaatcagttaaagaagctcaaagaaatctgtgagaaagaaaagaaagaattaaagaagaaaatggataaaaagaggcaggagaagataacagaagctaaatccaaagacaaaagtcagatggaagaggagaagacagagatgatccggtcatatatccaggaagtggtgcagtatatcaagaggctagaagaagcgcaaagtaaacggcaagaaaaactcgtagagaaacacaaggaaatacgtcagcagatcctggatgaaaagcccaagctgcaggtggagctggagcaagaataccaagacaaattcaaaagactgcccctcgagattttggaattcgtgcaggaagccatgaaaggaaagatcagtgaagacagcaatcacggttctgcccctctctccctgtcctcagaccctggaaaagtgaaccacaagactccctccagtgaggagctgggaggagacatcccaggaaaagaatttgatactcctctgtgaatgctcctg

SEQ ID NO: 4 - protein, 1216 aa, Homo sapiens
  phosphoinositide-specific phospholipase C beta 1, isoform a (PLC-beta1), transcript variant 1
MAGAQPGVHALQLKPVCVSDSLKKGTKFVKWDDDSTIVTPIILRTDPQGFFFYWTDQNKETELLDLSLVKDARCGRHAKAPKDPKLRELLDVGNIGRLEQRMITVVYGPDLVNISHLNLVAFQEEVAKEWTNEVFSLATNLLAQNMSRDAFLEKAYTKLKLQVTPEGRIPLKNIYRLFSADRKRVETALEACSLPSSRNDSIPQEDFTPEVYRVFLNNLCPRPEIDNIFSEFGAKSKPYLTVDQMMDFINLKQRDPRLNEILYPPLKQEQVQVLIEKYEPNNSLARKGQISVDGFMRYLSGEENGVVSPEKLDLNEDMSQPLSHYFINSSHNTYLTAGQLAGNSSVEMYRQVLLSGCRCVELDCWKGRTAEEEPVITHGFTMTTEISFKEVIEAIAECAFKTSPFPILLSFENHVDSPKQQAKMAEYCRLIFGDALLMEPLEKYPLESGVPLPSPMDLMYKILVKNKKKSHKSSEGSGKKKLSEQASNTYSDSSSMFEPSSPGAGEADTESDDDDDDDDCKKSSMDEGTAGSEAMATEEMSNLVNYIQPVKFESFEISKKRNKSFEMSSFVETKGLEQLTKSPVEFVEYNKMQLSRIYPKGTRVDSSNYMPQLFWNAGCQMVALNFQTMDLAMQINMGMYEYNGKSGYRLKPEFMRRPDKHFDPFTEGIVDGIVANTLSVKIISGQFLSDKKVGTYVEVDMFGLPVDTRRKAFKTKTSQGNAVNPVWEEEPIVFKKVVLPTLACLRIAVYEEGGKFIGHRILPVQAIRPGYHYICLRNERNQPLTLPAVFVYIEVKDYVPDTYADVIEALSNPIRYVNLMEQRAKQLAALTLEDEEEVKKEADPGETPSEAPSEARTTPAENGVNHTTTLTPKPPSQALHSQPAPGSVKAPAKTEDLIQSVLTEVEAQTIEELKQQKSFVKLQKKHYKEMKDLVKRHHKKTTDLIKEHTTKYNEIQNDYLRRRAALEKSAKKDSKKKSEPSSPDHGSSTIEQDLAALDAEMTQKLIDLKDKQQQQLLNLRQEQYYSEKYQKREHIKLLIQKLTDVAEECQNNQLKKLKEICEKEKKELKKKMDKKRQEKITEAKSKDKSQMEEEKTEMIRSYIQEVVQYIKRLEEAQSKRQEKLVEKHKEIRQQILDEKPKLQVELEQEYQDKFKRLPLEILEFVQEAMKGKISEDSNHGSAPLSLSSDPGKVNHKTPSSEELGGDIPGKEFDTPL

SEQ ID NO: 5 - DNA, 3052 bp, Homo sapiens
  cytoplasmic tyrosine kinase focal adhesion kinase (FAK)
ccggtgtgaaggccatgagtgattactgggttgttggaaagaagtctaactatgaagtattagaaaaagatgttggtttaaagcgattttttcctaagagtttactggattctgtcaaggccaaaacactaagaaaactgatccaacaaacatttagacaatttgccaaccttaatagagaagaaagtattctgaaattctttgagatcctgtctccagtctacagatttgataaggaatgcttcaagtgtgctcttggttcaagctggattatttcagtggaactggcaatcggcccagaagaaggaatcagttacctaacggacaagggctgcaatcccacacatcttgctgacttcactcaagtgcaaaccattcagtattcaaacagtgaagacaaggacagaaaaggaatgctacaactaaaaatagcaggtgcacccgagcctctgacagtgacggcaccatccctaaccattgcggagaatatggctgacctaatagatgggtactgccggctggtgaatggaacctcgcagtcatttatcatcagacctcagaaagaaggtgaacgggctttgccatcaataccaaagttggccaacagcgaaaagcaaggcatgcggacacacgccgtctctgtgtcagaaacagatgattatgctgagattatagatgaagaagatacttacaccatgccctcaaccagggattatgagattcaaagagaaagaatagaacttggacgatgtattggagaaggccaatttggagatgtacatcaaggcatttatatgagtccagagaatccagctttggcggttgcaattaaaacatgtaaaaactgtacttcggacagcgtgagagagaaatttcttcaagaagcctgccattacacatctttgcactggaattggtgcagatatataagtgatcctaatgttgatgcctgcccagaccccaggaatgcagagttaacaatgcgtcagtttgaccatcctcatattgtgaagctgattggagtcatcacagagaatcctgtctggataatcatggagctgtgcacacttggagagctgaggtcatttttgcaagtaaggaaatacagtttggatctagcatctttgatcctgtatgcctatcagcttagtacagctcttgcatatctagagagcaaaagatttgtacacagggacattgctgctcggaatgttctggtgtcctcaaatgattgtgtaaaattaggagactttggattatcccgatatatggaagatagtacttactacaaagcttccaaaggaaaattgcctattaaatggatggctccagagtcaatcaattttcgacgttttacctcagctagtgacgtatggatgtttggtgtgtgtatgtgggagatactgatgcatggtgtgaagccttttcaaggagtgaagaacaatgatgtaatcggtcgaattgaaaatggggaaagattaccaatgcctccaaattgtcctcctaccctctacagccttatgacgaaatgctgggcctatgaccccagcaggcggcccaggtttactgaacttaaagctcagctcagcacaatcctggaggaagagaaggctcagcaagaagagcgcatgaggatggagtccagaagacaggccacagtgtcctgggactccggagggtctgatgaagcaccgcccaagcccagcagaccgggttatcccagtccgaggtccagcgaaggattttatcccagcccacagcacatggtacaaaccaatcattaccaggtttctggctaccctggttcacatggaatcacagccatggctggcagcatctatccaggtcaggcatctcttttggaccaaacagattcatggaatcatagatctcaggagatagcaatgtggcagcccaatgtggaggactctacagtattggacctgcgagggattgggcaagtgttgccaacccatctgatggaagagcgtctaatccgacagcaacaggaaatggaagaagatcagcgctggctggaaaaagaggaaagatttctgattggaaaccaacatatatatcagcctgtgggtaaaccagatcctgcagctccaccaaagaaaccgcctcgccctggagctcccggtcatctgggaagccttgccagcctcagcagccctgctgacagctacaacgagggtgtcaagcttcagccccaggaaatcagcccccctcctactgccaacctggaccggtcgaatgataaggtgtacgagaatgtgacgggcctggtgaaagctgtcatcgagatgtccagtaaaatccagccagccccaccagaggagtatgtccctatggtgaaggaagtcggcttggccctgaggacattattggccactgtggatgagaccattcccctcctaccagccagcacccaccgagagattgagatggcacagaagctattgaactctgacctgggtgagctcatcaacaagatgaaactggcccagcagtatgtcatgaccagcctccagcaagagtacaaaaagcaaatgctgactgccgctcacgccctggctgtggatgccaaaaacttactcgatgtcattgaccaagcaagactgaaaatgcttgggcagacgagaccacactgagcctcccctaggagcacgtcttgctaccctcttttgaagatgttctctagccttccaccagcagcgaggaattaaccctgtgtcctcagtcgccagcactcacagctccaacttttttgaatgaccatctggttgaaaaatctttctcatataagtttaaccacactttgatttgggttcattttttgttttgtttttttcaatcatgatattcagaaaaatccaggatccaaaatgtggcgtttttctaagaatgaaaattatatgtaagcttttaagcatcatgaagaacaatttatgttcacattaagatacgttctaaagggggatggccaaggggtgacatcttaattcctaaactaccttagctgcatagtggaagaggagagccggaattc

SEQ ID NO: 6 - protein, 879 aa, Homo sapiens
  cytoplasmic tyrosine kinase focal adhesion kinase (FAK)
MSDYWVVGKKSNYEVLEKDVGLKRFFPKSLLDSVKAKTLRKLIQQTFRQFANLNREESILKFFEILSPVYRFDKECFKCALGSSWIISVELAIGPEEGISYLTDKGCNPTHLADFTQVQTIQYSNSEDKDRKGMLQLKIAGAPEPLTVTAPSLTIAENMADLIDGYCRLVNGTSQSFIIRPQKEGERALPSIPKLANSEKQGMRTHAVSVSETDDYAEIIDEEDTYTMPSTRDYEIQRERIELGRCIGEGQFGDVHQGIYMSPENPALAVAIKTCKNCTSDSVREKFLQEACHYTSLHWNWCRYISDPNVDACPDPRNAELTMRQFDHPHIVKLIGVITENPVWIIMELCTLGELRSFLQVRKYSLDLASLILYAYQLSTALAYLESKRFVHRDIAARNVLVSSNDCVKLGDFGLSRYMEDSTYYKASKGKLPIKWMAPESINFRRFTSASDVWMFGVCMWEILMHGVKPFQGVKNNDVIGRIENGERLPMPPNCPPTLYSLMTKCWAYDPSRRPRFTELKAQLSTILEEEKAQQEERMRMESRRQATVSWDSGGSDEAPPKPSRPGYPSPRSSEGFYPSPQHMVQTNHYQVSGYPGSHGITAMAGSIYPGQASLLDQTDSWNHRSQEIAMWQPNVEDSTVLDLRGIGQVLPTHLMEERLIRQQQEMEEDQRWLEKEERFLIGNQHIYQPVGKPDPAAPPKKPPRPGAPGHLGSLASLSSPADSYNEGVKLQPQEISPPPTANLDRSNDKVYENVTGLVKAVIEMSSKIQPAPPEEYVPMVKEVGLALRTLLATVDETIPLLPASTHREIEMAQKLLNSDLGELINKMKLAQQYVMTSLQQEYKKQMLTAAHALAVDAKNLLDVIDQARLKMLGQTRPH

SEQ ID NO: 7 - DNA, 4089 bp, Homo sapiens
  calcium dependent tyrosine kinase focal adhesion kinase 2 (FAK2)
gaattccgtcagcccttttactcagccacagcctccggagccgttgcacacctacctgcccggccgacttacctgtacttgccgccgtcccggctcacctggcggtgcccgaggagtagtcgctggagtccgcgcctccctgggactgcaatgtgccgatcttagctgctgcctgagaggatgtctggggtgtccgagcccctgagtcgagtaaagttgggcacgttacgccggcctgaaggccctgcagagcccatggtggtggtaccagtagatgtggaaaaggaggacgtgcgtatcctcaaggtctgcttctatagcaacagcttcaatcctgggaaaaacttcaaactggtcaaatgcactgtccagacggagatccgggagatcatcacctccatcctgctgagcgggcggatcgggcccaacatccggttggctgagtgctatgggctgaggctgaagcacatgaagtccgatgagatccactggctgcacccacagatgacggtgggtgaggtgcaggacaagtatgagtgtctgcacgtggaagccgagtggaggtatgaccttcaaatccgctacttgccagaagacttcatggagagcctgaaggaggacaggaccacgctgctctatttttaccaacagctccggaacgactacatgcagcgctacgccagcaaggtcagcgagggcatggccctgcagctgggctgcctggagctcaggcggttcttcaaggatatgccccacaatgcacttgacaagaagtccaacttcgagctcctagaaaaggaagtggggctggacttgtttttcccaaagcagatgcaggagaacttaaagcccaaacagttccggaagatgatccagcagaccttccagcagtacgcctcgctcagggaggaggagtgcgtcatgaagttcttcaacactctcgccccgttcgccaacatcgaccaggagacctaccgctgtgaactcattcaaggatggaacattactgtggacctggtcattggccctaaagggatccgccagctgactagtcaggacgcaaagcccacctgcctggccgagttcaagcagatcaggtccatcaggtgcctcccgctggaggagggccaggcagtacttcagctgggcattgaaggtgccccccaggccttgtccatcaaaacctcatccctagcagaggctgagaacatggctgacctcatagacggctactgccggctgcagggtgagcaccaaggctctctcatcatccatcctaggaaagatggtgagaagcggaacagcctgccccagatccccatgctaaacctggaggcccggcggtcccacctctcagagagctgcagcatagagtcagacatctacgcagagattcccgacgaaaccctgcgaaggcccggaggtccacagtatggcattgcccgtgaagatgtggtcctgaatcgtattcttggggaaggcttttttggggaggtctatgaaggtgtctacacaaatcataaaggggagaaaatcaatgtagctgtcaagacctgcaagaaagactgcactctggacaacaaggagaagttcatgagcgaggcagtgatcatgaagaacctcgaccacccgcacatcgtgaagctgatcggcatcattgaagaggagcccacctggatcatcatggaattgtatccctatggggagctgggccactacctggagcggaacaagaactccctgaaggtgctcaccctcgtgctgtactcactgcagatatgcaaagccatggcctacctggagagcatcaactgcgtgcacagggacattgctgtccggaacatcctggtggcctcccctgagtgtgtgaagctgggggactttggtctttcccggtacattgaggacgaggactattacaaagcctctgtgactcgtctccccatcaaatggatgtccccagagtccattaacttccgacgcttcacgacagccagtgacgtctggatgttcgccgtgtgcatgtgggagatcctgagctttgggaagcagcccttcttctggctggagaacaaggatgtcatcggggtgctggagaaaggagaccggctgcccaagcctgatctctgtccaccggtcctttataccctcatgacccgctgctgggactacgaccccagtgaccggccccgcttcaccgagctggtgtgcagcctcagtgacgtttatcagatggagaaggacattgccatggagcaagagaggaatgctcgctaccgaacccccaaaatcttggagcccacagccttccaggaacccccacccaagcccagccgacctaagtacagaccccctccgcaaaccaacctcctggctccaaagctgcagttccaggttcctgagggtctgtgtgccagctctcctacgctcaccagccctatggagtatccatctcccgttaactcactgcacaccccacctctccaccggcacaatgtcttcaaacgccacagcatgggggaggaggacttcatccaacccagcagccgagaagaggcccagcagctgtgggaggctgaaaaggtcaaaatgcggcaaatcctggacaaacagcagaagcagatggtggaggactaccagtggctcaggcaggaggagaagtccctggaccccatggtttatatgaatgataagtccccattgacgccagagaaggaggtcggctacctggagttcacagggcccccacagaagcccccgaggctgggcgcacagtccatccagcccacagctaacctggaccggaccgatgacctggtgtacctcaatgtcatggagctggtgcgggccgtgctggagctcaagaatgagctctgtcagctgccccccgagggctacgtggtggtggtgaagaatgtggggctgaccctgcggaagctcatcgggagcgtggatgatctcctgccttccttgccgtcatcttcacggacagagatcgagggcacccagaaactgctcaacaaagacctggcagagctcatcaacaagatgcggctggcgcagcagaacgccgtgacctccctgagtgaggagtgcaagaggcagatgctgacggcttcacacaccctggctgtggacgccaagaacctgctcgacgctgtggaccaggccaaggttctggccaatctggcccacccacctgcagagtgacggagggtgggggccacctgcctgcgtcttccgcccctgcctgccatgtacctcccctgccttgctgttggtcatgtgggtcttccagggagaaggccaaggggagtcaccttcccttgccactttgcacgacgccctctccccacccctacccctggctgtactgctcaggctgcagctggacagaggggactctgggctatggacacagggtgacggtgacaaagatggctcagagggggactgctgctgcctggccactgctccctaagccagcctggtccatgcagggggctcctgggggtggggaggtgtcacatggtgcccctagctttatatatggacatggcaggccgatttgggaaccaagctattcctttcccttcctcttctcccctcagatgtcccttgatgcacagagaagctggggaggagctttgttttcgggggtcaggcagccagtgagatgagggatgggcctggcattcttgtacagtgtatattgaaatttatttaatgtgaggtttggtctggactgacagcatgtgccctcctgagggaggaccagggcacagtccaggaacaagctaattgggagtccaggcacaggatgctgtgttgtcaacaaaccaagcatcagggggaagaagcagagagatgcggccaagataggaccttgggccaaatccgctctcttcctgcccctctttctctttcttcctttactttcccttgcttttccctcttttcttactcctcctctttctctcccccacccccattctcatctgcacccttcttttctcatgtgtttgcataaacattcttttaacttctttctatttgacttgtggttgaattaaaattgtcccatttgca

SEQ ID NO: 8 - protein, 1009 aa, Homo sapiens
  calcium dependent tyrosine kinase focal adhesion kinase 2 (FAK2)
MSGVSEPLSRVKLGTLRRPEGPAEPMVVVPVDVEKEDVRILKVCFYSNSFNPGKNFKLVKCTVQTEIREIITSILLSGRIGPNIRLAECYGLRLKHMKSDEIHWLHPQMTVGEVQDKYECLHVEAEWRYDLQIRYLPEDFMESLKEDRTTLLYFYQQLRNDYMQRYASKVSEGMALQLGCLELRRFFKDMPHNALDKKSNFELLEKEVGLDLFFPKQMQENLKPKQFRKMIQQTFQQYASLREEECVMKFFNTLAGFANIDQETYRCELIQGWNITVDLVIGPKGIRQLTSQDAKPTCLAEFKQIRSIRCLPLEEGQAVLQLGIEGAPQALSIKTSSLAEAENMADLIDGYCRLQGEHQGSLIIHPRKDGEKRNSLPQIPMLNLEARRSHLSESCSIESDIYAEIPDETLRRPGGPQYGIAREDVVLNRILGEGFFGEVYEGVYTNHKGEKINVAVKTCKKDCTLDNKEKFMSEAVIMKNLDHPHIVKLIGIIEEEPTWIIMELYPYGELGHYLERNKNSLKVLTLVLYSLQICKAMAYLESINCVHRDIAVRNILVASPECVKLGDFGLSRYIEDEDYYKASVTRLPIKWMSPESINFRRFTTASDVWMFAVCMWEILSFGKQPFFWLENKDVIGVLEKGDRLPKPDLCPPVLYTLMTRCWDYDPSDRPRFTELVCSLSDVYQMEKDIAMEQERNARYRTPKILEPTAFQEPPPKPSRPKYRPPPQTNLLAPKLQFQVPEGLCASSPTLTSPMEYPSPVNSLHTPPLHRHNVFKRHSMREEDFIQPSSREEAQQLWEAEKVKMRQILDKQQKQMVEDYQWLRQEEKSLDPMVYMNDKSPLTPEKEVGYLEFTGPPQKPPRLGAQSIQPTANLDRTDDLVYLNVMELVRAVLELKNELCQLPPEGYVVVVKNVGLTLRKLIGSVDDLLPSLPSSSRTEIEGTQKLLNKDLAELINKMRLAQQNAVTSLSEECKRQMLTASHTLAVDAKNLLDAVDQAKVLANLAHPPAE

SEQ ID NO: 9 - DNA, 2195 bp, Homo sapiens
  serine threonine protein kinase casein kinase 2, alpha 1 subunit isoform a, transcript variant 2 (CK2, CK2alpha), CK2 catalytic subunit alpha
aggggagagcggccgccgccgctgccgcttccaccacagtttgaagaaaacaggtctgaaacaaggtcttacccccagctgcttctgaacacagtgactgccagatctccaaacatcaagtccagctttgtccgccaacctgtctgacatgtcgggacccgtgccaagcagggccagagtttacacagatgttaatacacacagacctcgagaatactgggattacgagtcacatgtggtggaatggggaaatcaagatgactaccagctggttcgaaaattaggccgaggtaaatacagtgaagtatttgaagccatcaacatcacaaataatgaaaaagttgttgttaaaattctcaagccagtaaaaaagaagaaaattaagcgtgaaataaagattttggagaatttgagaggaggtcccaacatcatcacactggcagacattgtaaaagaccctgtgtcacgaacccccgccttggtttttgaacacgtaaacaacacagacttcaagcaattgtaccagacgttaacagactatgatattcgattttacatgtatgagattctgaaggccctggattattgtcacagcatgggaattatgcacagagatgtcaagccccataatgtcatgattgatcatgagcacagaaagctacgactaatagactggggtttggctgagttttatcatcctggccaagaatataatgtccgagttgcttcccgatacttcaaaggtcctgagctacttgtagactatcagatgtacgattatagtttggatatgtggagtttgggttgtatgctggcaagtatgatctttcggaaggagccatttttccatggacatgacaattatgatcagttggtgaggatagccaaggttctggggacagaagatttatatgactatattgacaaatacaacattgaattagatccacgtttcaatgatatcttgggcagacactctcgaaagcgatgggaacgctttgtccacagtgaaaatcagcaccttgtcagccctgaggccttggatttcctggacaaactgctgcgatatgaccaccagtcacggcttactgcaagagaggcaatggagcacccctatttctacactgttgtgaaggaccaggctcgaatgggttcatctagcatgccagggggcagtacgcccgtcagcagcgccaatatgatgtcagggatttcttcagtgccaaccccttcaccccttggacctctggcaggctcaccagtgattgctgctgccaacccccttgggatgcctgttccagctgccgctggcgctcagcagtaacggccctatctgtctcctgatgcctgagcagaggtgggggagtccaccctctccttgatgcagcttgcgcctggcggggaggggtgaaacacttcagaagcaccgtgtctgaaccgttgcttgtggatttatagtagttcagtcataaaaaaaaaattataataggctgattttcttttttctttttttttttaactcgaacttttcataactcaggggattccctgaaaaattacctgcaggtggaatatttcatggacaaatttttttttctcccctcccaaatttagttcctcatcacaaaagaacaaagataaaccagcctcaatcccggctgctgcatttaggtggagacttcttcccattcccaccattgttcctccaccgtcccacactttagggggttggtatctcgtgctcttctccagagattacaaaaatgtagcttctcaggggaggcaggaagaaaggaaggaaggaaagaaggaagggaggacccaatctataggagcagtggactgcttgctggtcgcttacatcactttactccataagcgcttcagtggggttatcctagtggctcttgtggaagtgtgtcttagttacatcaagatgttgaaaatctacccaaaatgcagacagatactaaaaacttctgttcagtaagaatcatgtcttactgatctaaccctaaatccaactcatttatacttttatttttagttcagtttaaaatgttgataccttccctcccaggctccttaccttggtcttttccctgttcatctcccaacatgctgtgctccatagctggtaggagagggaaggcaaaatctttcttagttttctttgtcttggccattttgaattc

SEQ ID NO: 10 - protein, 391 aa, Homo sapiens
  serine threonine protein kinase casein kinase 2, alpha 1 subunit isoform a, transcript variant 2 (CK2, CK2alpha), CK2 catalytic subunit alpha
MSGPVPSRARVYTDVNTHRPREYWDYESHVVEWGNQDDYQLVRKLGRGKYSEVFEAINITNNEKVVVKILKPVKKKKIKREIKILENLRGGPNIITLADIVKDPVSRTPALVFEHVNNTDFKQLYQTLTDYDIRFYMYEILKALDYCHSMGIMHRDVKPHNVMIDHEHRKLRLIDWGLAEFYHPGQEYNVRVASRYFKGPELLVDYQMYDYSLDMWSLGCMLASMIFRKEPFFHGHDNYDQLVRIAKVLGTEDLYDYIDKYNIELDPRFNDILGRHSRKRWERFVHSENQHLVSPEALDFLDKLLRYDHQSRLTAREAMEHPYFYTVVKDQARMGSSSMPGGSTPVSSANMMSGISSVPTPSPLGPLAGSPVIAAANPLGMPVPAAAGAQQ

SEQ ID NO: 11 - DNA, 4626 bp, Homo sapiens
  cMET proto-oncogene tyrosine kinase
gaattccgccctcgccgcccgcggcgccccgagcgctttgtgagcagatgcggagccgagtggagggcgcgagccagatgcggggcgacagctgacttgctgagaggaggcggggaggcgcggagcgcgcgtgtggtccttgcgccgctgacttctccactggttcctgggcaccgaaagataaacctctcataatgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtacttggtggaaagaacctctcaacattgtcagttttctattttgctttgccagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaagcgatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtcgtcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatcatagtgctagtactatgtcaaagcaacagtccacactttgtccaatggttttttcactgcctgacctttaaaaggccatcgatattctttgctccttgccataggacttgtattgttatttaaattactggattctaaggaatttcttatctgacagagcatcagaaccagaggcttggtcccacaggccagggaccaatgcgctgcag

SEQ ID NO: 12 - protein, 1408 aa, Homo sapiens
  cMET proto-oncogene tyrosine kinase
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKAMKYLASKKFVHRDLAARNCMLDEKFTVKVADFGLARDMYDKEYYSVHNKTGAKLPVKWMALESLQTQKFTTKSDVWSFGVVLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 13 - DNA, 3350 bp, Homo sapiens
  flap structure-specific endonuclease 1 (FEN1) 5′-3′ exonuclease
cacagtccactctgtcagggtttaaggcaggaaaaacatgctcattttgatggtaatattcttccttctcaacattccatttctcctggcaaatttcatggatcccagatgcttttggaaaataaatttgaatgaaatcaaggatgaagtccttgggatgacttgttccttcatccttgaaacagttcagaagactatggacaaagattatttcaaccagactctgaatgtcctaaatacaactacaaaccacaaatatgccttggcattggcctttacagtggatgaaatcaacaggaatcctgatcttttaccaaatatgtctctgattataaaatacaatttgggtcattgtgatggaaaaactgtaacaactctatccgatttatttaatccaaataatcatctccatttccccaattatttatgtaatgaagggattatgtgtttggttctgcttacaggaccacattggagagcatctttatatctctggatatccgtgtatgtctacctgtctccacatttccttcagctttcctatggacctttctactccatcttcagtgataatgaacaatatccttatctctatcagatgggcccaaaggactcatcactagcattggcaatggtctccttcataatttacttcaagtggaactgggttgggctatttatctcagatgatgatcaaggcaatcaatttctctcagagttgaaaaaagagagccaaaccaaggatatttgctttgcctttgtgaacatgatatcagtcagtgatgtttcatactatcataaaactgaaatgtactacaaccaaattgtgatgtcatccacaaaggttattatcatttatggggaaacaaacagtattattgaattgagcttcagaatgtggtcatctccagttaaacagagaatatgggtcaccacaaaacaatttgattgccctaccagtaagagagacttaactcatggcacattctatgggacccttacatttctacaccactatggtgagatttctggctttaaaaattttgtacagacacggtacaatctcagaagcacagatttatatctagtaatgccagagtggaaatattttaactatgaagcctcagcatctaactgtaaaatactgagaaactatttatccaatatctcactggaatggctaatggaacagaaatttgacatgtcatttagtgattatagtcacaacatatacaatgctgtatatgccattgctcatgcactccatgagaagaatctgcaagaagttgaaaatcaggcaataaacaatgcgaaaggagaaaatactcactgcttgaagctaaactcatttctgagaaagacccacttcactaattctcttgggaacagagtaattatgaaacagagagaagtagtgcatggagactataatattgttcacatgtggaatttctcacaacgccttgggattaaggtgaagataggacaattcagcccacattttccacagggtcaacagttacacttatatgtagacatgactgagttggctacaggaagtagaaagatgccatcctcagtgtgcagtgcagattgccatcctggattcagaagaatctggaaggaggaaatggcagcctgctgttttgtttgcaacccctgccctgaaaatgaaatttctaatgagacgatggtggtattttgggtcttcgtgaagcaccatgacactcctattgtgaaggccaataacagaatcctcagctacctattaatcgtgtcactcatgttctgttttctgtgctcctttttcttcattggctatcctaacagagcaacctgtatcttacagcaaatcacatttggaatcttctttactgtggctatttccacagttctggccaaaacaatcactgtggttctggctttcaaagtcacagacccaggaagacaattaagaatctttttggtatcggggacacccaactacattattcccatatgttccctattgcaatgtattctgtgtgcaatctggctagcagtttctcctccctttgttgatattgatgaacactctgagcatggccacatcatcattgtgtgcaacaagggctccattactgcattctactgtgtcctgggatacttggcctgcctggcctttggaagcttcactatagctttcttggcaaagaacctgcctgacacattcaacgaagccaagttcttgaccttcagcatgctagtgttctgcgctgtctgggtcaccttcctccctgtctaccatagcaccaagggcaaggtcatggttgctgtggagatcttctccatcttggcatctagtgcagggatgctgggatgcatctttgcacccaaagtttacatcattttaatgagaccagacagaaattcgatccacaaaatcagggagaaatcatatttctgaaaaggtatttcaggaattctgtcaaatgtaaagttgatacatacaccccaaatatttagttacagagcatatatctagttttagaatcactctcactggttcctctagttaagcatagaagtaccatatgtactgatcttgcatatgttgtctataaaatcttacaatcattcatttgcttagtatcttctggaagaagtaaaattttcaaataactagtacaattttattcattattttgctttcatgaggatttccccctggtaacttcaaataaattttataagtcagttgaatatataaccttacatagaaagtgagttctaggacagacagggattatacatagaaacaaactaactaaaaatcaacaaagatgaaatcagaacacattttcttatttccagtaggaacacatacttgacagaatactgtctttttttcagctgctctttaagatattggccaatagtctaagctgaaaatgttctttatctactctcaaatacaaaaatattatatccaacaatggacagaatctgagaactcctgtggttgagttagggaatagttggaagatactgagaaggaggtgacccataggaatacaaagcagtctcaactaacctggacaaccaaggtccctcagacactgagccactaacaagtcagcctactccagctgttatgaggcccccaaaacatatgcaacataggattgcctggtccagcctcagcaagagaatacacacctaaccacagagagacttccccaagggattggggaggtctggggtttggagagttgcggattgtcccttgatgattggaaggaggtattggatgagaatgaatcagggggaagactaggaaggggataatgatggaactgtaaaaaaaaaaa

SEQ ID NO: 14 - protein, 380 aa, Homo sapiens
  flap structure-specific endonuclease 1 (FEN1) 5′-3′ exonuclease
MGIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVKAGKVYAAATEDMDCLTFGSPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQGSTQGRLDDFFKVTGSLSSAKRKEPEPKGSTKKKAKTGAAGKFKRGK

SEQ ID NO: 15 - DNA, 4276 bp, Homo sapiens
  REV1 dCMP transferase
agagccaccgcggagcgcgcgcggggttggttgccgcgagcgtgggggagcgtggaccgcggcgctgctcagcggtggggctgccttcccccggccctcctccctggtccctggcgagggcactggcggcggcggggccggggtccgcaaggccggagaaggccgccgggcccgggcatggtggtctggggcaacgcggaagaagctccaccatgaggcgaggtggatggaggaagcgagctgaaaatgatggctgggaaacatggggtgggtatatggctgccaaggtccagaaattggaggaacagtttcgatcagatgctgctatgcagaaggatgggacttcatctacaatttttagtggagttgccatctatgttaatggatacacagatccttccgctgaggaattgagaaaactaatgatgttgcatggaggtcaataccatgtatattattccagatctaaaacaacacatattattgccacaaatcttcccaatgccaaaattaaagaattaaagggggaaaaagtaattcgaccagaatggattgtggaaagcatcaaagctggacgactcctctcctacattccatatcagctgtacaccaagcagtccagtgtgcagaaaggtctcagctttaatcctgtatgcagacctgaggatcctctgccaggtccaagcaatatagccaaacagctcaacaacagggtaaatcacatcgttaagaagattgaaacggaaaatgaagtcaaagtcaatggcatgaacagttggaatgaagaagatgaaaataatgattttagttttgtggatctggagcagacctctccgggaaggaaacagaatggaattccgcatcccagagggagcactgccatttttaatggacacactcctagctctaatggtgccttaaagacacaggattgcttggtgcccatggtcaacagtgttgccagcaggctttctccagccttttcccaggaggaggataaggctgagaagagcagcactgatttcagagactgcactctgcagcagttgcagcaaagcaccagaaacacagatgctttgcggaatccacacagaactaattctttctcattatcacctttgcacagtaacactaaaatcaatggtgctcaccactccactgttcaggggccttcaagcacaaaaagcacttcttcagtatctacgtttagcaaggcagcaccttcagtgccatccaaaccttcagactgcaattttatttcaaacttctattctcattcaagactgcatcacatatcaatgtggaagtgtgaattgactgagtttgtcaataccctacaaagacaaagtaatggtatctttccaggaagggaaaagttaaaaaaaatgaaaacaggcaggtctgcacttgttgtaactgacacaggagatatgtcagtattgaattctcccagacatcagagctgtataatgcatgttgatatggattgcttctttgtatcagtgggtatacgaaatagaccagatctcaaaggaaaaccagtggctgttacaagtaacagaggcacaggaagggcacctttacgtcctggcgctaacccccagctggagtggcagtattaccagaataaaatcctgaaaggcaaagcagcagatataccagattcatcattgtgggagaatccagattctgcgcaagcaaatggaattgattctgttttgtcaagggctgaaattgcatcttgtagttatgaggccaggcaacttggcattaagaacggaatgttttttgggcatgctaaacaactatgtcctaatcttcaagctgttccatacgattttcatgcatataaggaagtcgcacaaacattgtatgaaacattggcaagctacactcataacattgaagctgtcagttgtgatgaagcgctggtagacattaccgaaatccttgcagagaccaaacttactcctgatgaatttgcaaatgctgttcgtatggaaatcaaagaccagacgaaatgtgctgcctctgttggaattggttctaatattctcctggctagaatggcaactagaaaagcaaaaccagatgggcagtaccacctaaaaccagaagaagtagatgattttatcagaggccagctagtgaccaatctaccaggagttggacattcaatggaatctaagttggcatctttgggaattaaaacttgtggagacttgcagtatatgaccatggcaaaactccaaaaagaatttggtcccaaaacaggtcagatgctttataggttctgccgtggcttggatgatagaccagttcgaactgaaaaggaaagaaaatctgtttcagctgagatcaactatggaataaggtttactcagccaaaagaggcagaagcttttcttctgagtctttcagaagaaattcaaagaagactagaagccactggcatgaagggtaaacgtctaactctcaaaatcatggtacgaaagcctggggctcctgtagaaactgcaaaatttggaggccatggaatttgtgataacattgccaggactgtaactcttgaccaggcaacagataatgcaaaaataattggaaaggcgatgctaaacatgtttcatacaatgaaactaaatatatcagatatgagaggggttgggattcacgtgaatcagttggttccaactaatctgaacccttccacatgtcccagtcgcccatcagttcagtcaagccactttcctagtgggtcatactctgtccgtgatgtcttccaagttcagaaagctaagaaatccaccgaagaggagcacaaagaagtatttcgggctgctgtggatctggaaatatcatctgcttctagaacttgcactttcttgccaccttttcctgcacatctgccgaccagtcctgatactaacaaggctgagtcttcagggaaatggaatggtctacatactcctgtcagtgtgcagtcgagacttaacctgagtatagaggtcccgtcaccttcccagctggatcagtctgttttagaagcacttccacctgatctccgggaacaagtagagcaagtctgtgctgtccagcaagcagagtcacatggcgacaaaaagaaagaaccagtaaatggctgtaatacaggaattttgccacaaccagttgggacagtcttgttgcaaataccagaacctcaagaatcgaacagtgacgcaggaataaatttaatagcccttccagcattttcacaggtggaccctgaggtatttgctgcccttcctgctgaacttcagagggagctgaaagcagcgtatgatcaaagacaaaggcagggcgagaacagcactcaccagcagtcagccagcgcatctgtgccaaagaatcctttacttcatctaaaggcagcagtgaaagaaaagaaaagaaacaagaagaaaaaaaccattggttcaccaaaaaggattcagagtcctttgaataacaagctgcttaacagtcctgcaaaaactctgccaggggcctgtggcagtccccagaagttaattgatgggtttctaaaacatgaaggacctcctgcagagaaacccctggaagaactctctgcttctacttcaggtgtgccaggcctttctagtttgcagtctgacccagctggctgtgtgagacctccagcacccaatctagctggagctgttgaattcaatgatgtgaagaccttgctcagagaatggataactacaatttcagatccaatggaagaagacattctccaagttgtgaaatactgtactgatctaatagaagaaaaagatttggaaaaactggatctagttataaaatacatgaaaaggctgatgcagcaatcggtggaatcggtttggaatatggcatttgactttattcttgacaatgtccaggtggttttacaacaaacttatggaagcacattaaaagttacataaatattaccagagagcctgatgctctctgatagctgtgccataagtgcttgtgaggtatttgcaaagtgcatgatagtaatgctcggagtttttataattttaaatttcttttaaagcaagtgttttgtacatttcttttcaaaaagtgccaaatttgtcagtattgcatgtaaataattgtgttaattattttactgtagcatagattctatttacaaaatgtttgtttataaagttttatggatttttacagtgaagtgtttacagttgtttaataaagaactgtatgtaaaaaaaaaaaaaaaaaa

SEQ ID NO: 16 - protein, 1251 aa, Homo sapiens
  REV1 dCMP transferase
MRRGGWRKRAENDGWETWGGYMAAKVQKLEEQFRSDAAMQKDGTSSTIFSGVAIYVNGYTDPSAEELRKLMMLHGGQYHVYYSRSKTTHIIATNLPNAKIKELKGEKVIRPEWIVESIKAGRLLSYIPYQLYTKQSSVQKGLSFNPVCRPEDPLPGPSNIAKQLNNRVNHIVKKIETENEVKVNGMNSWNEEDENNDFSFVDLEQTSPGRKQNGIPHPRGSTAIFNGHTPSSNGALKTQDCLVPMVNSVASRLSPAFSQEEDKAEKSSTDFRDCTLQQLQQSTRNTDALRNPHRTNSFSLSPLHSNTKINGAHHSTVQGPSSTKSTSSVSTFSKAAPSVPSKPSDCNFISNFYSHSRLHHISMWKCELTEFVNTLQRQSNGIFPGREKLKKMKTGRSALVVTDTGDMSVLNSPRHQSCIMHVDMDCFFVSVGIRNRPDLKGKPVAVTSNRGTGRAPLRPGANPQLEWQYYQNKILKGKAADIPDSSLWENPDSAQANGIDSVLSRAEIASCSYEARQLGIKNGMFFGHAKQLCPNLQAVPYDFHAYKEVAQTLYETLASYTHNIEAVSCDEALVDITEILAETKLTPDEFANAVRMEIKDQTKCAASVGIGSNILLARMATRKAKPDGQYHLKPEEVDDFIRGQLVTNLPGVGHSMESKLASLGIKTCGDLQYMTMAKLQKEFGPKTGQMLYRFCRGLDDRPVRTEKERKSVSAEINYGIRFTQPKEAEAFLLSLSEEIQRRLEATGMKGKRLTLKIMVRKPGAPVETAKFGGHGICDNIARTVTLDQATDNAKIIGKAMLNMFHTMKLNISDMRGVGIHVNQLVPTNLNPSTCPSRPSVQSSHFPSGSYSVRDVFQVQKAKKSTEEEHKEVFRAAVDLEISSASRTCTFLPPFPAHLPTSPDTNKAESSGKWNGLHTPVSVQSRLNLSIEVPSPSQLDQSVLEALPPDLREQVEQVCAVQQAESHGDKKKEPVNGCNTGILPQPVGTVLLQIPEPQESNSDAGINLIALPAFSQVDPEVFAALPAELQRELKAAYDQRQRQGENSTHQQSASASVPKNPLLHLKAAVKEKKRNKKKKTIGSPKRIQSPLNNKLLNSPAKTLPGACGSPQKLIDGFLKHEGPPAEKPLEELSASTSGVPGLSSLQSDPAGCVRPPAPNLAGAVEFNDVKTLLREWITTISDPMEEDILQVVKYCTDLIEEKDLEKLDLVIKYMKRLMQQSVESVWNMAFDFILDNVQVVLQQTYGSTLKVT

SEQ ID NO: 17 - DNA, 2957 bp, Homo sapiens
  apyrimidinic endonuclease 1 (APE1), AP endonuclease 1, HAP1
ctgcagatagcactgggaaagacaccgcggaactcccgcgagcgagacccgccaaggcccctccagggacctgtcttcctaacgtccagggagcccgagccaactcgcgccttacattcgtatccgttttcctatctctttcccgtggtcagcccagccttctccactgtttttttcctcttgcacagagttagaatcttaagtcagtgtcacacaatgtgctgtgcatctggcacaacgataaacagccgagggagggttggggactaagtgcctagagaattagaggagggaggcgaggctaagcgtccgtcacgtggtgtcagacagaccaatcacgcgcattcttcggccacgacaagcgcgcctctgatcacgtgaccaggtccgctacccacgtgggggctcagcgtgcacccttctttgtgctcgggttaggaggagctaggctgccatcgggccggtgcagatacggggttgctcttttgctcataagaggggcttcgctggcagtctgaacggcaagcttgagtcaggacccttaattaagatcctcaattggctggagggcagatctcgcgagtagggtacaaggcactatgaaatgatctagtttcgtgggtgaggggctgaagggcctatgatgcacggaggcggggaaaggatttagagataacgtggtttaaaggcgggacctggtgcggggacgctccttgggaggagtcttctcccagccttagctggtttcatgatttctttgcgtctgtaggcaacgcggtaaaaatattgcttcggtgggtgacgcggtacagctgcccaagggcgttcgtaacgggaatgccgaagcgtgggaaaaagggagcggtggcggaagacggggatgagctcaggacaggtaagggaatgaaatcagcccttcttcctagaagctgcggcgggggtgtttgtcattcccttgatgtacggtaagtacgggccgactcatttttgcaggggtttgtgaagaagtcgcaggaaccgtaggctttcgttgggtctatagttaacgccggatcgcagttggaaaccaccagctttttgtcagtatatattactcattttatagagccagaggccaagaagagtaagacggccgcaaagaaaaatgacaaagaggcagcaggagagggcccagccctgtatgaggaccccccagatcagaaaacctcacccagtggcaaacctgccacactcaagatctgctcttggaatgtggatgggcttcgagcctggattaagaagaaaggattagatgtgagtggaatttgagggaaagagacattttttagtattgaatggtcttagggtttagtcaccccttttctccgtttagccttcaggctgttttatttttctcctgcccgtagttttctgtggggcttccccagtcttgccagttgtatttcctaaatgtctgttccttcacttccattgccattttcttttttagtgttctctcctcttcccagaatgttgcaaaaacctcttcactatacttcctccattttatcttcctgcattgcattccatatgaagcatgtcctccattccattaaccatagcttaaaatcttagcttgctatccactgcctatagaaaaaacacatctccttggcatagcatgtaagactttcttacctctctatatttgttttcatttatctagcttagaattgtttgaatattgtgctgcttgactcgaactccttaggccaagagactgtttaacccgtgcgtatctatgacttagcatatagattattcaataaatgttctgctgaattgataatacgttttccacctttcttttcacttacagtgggtaaaggaagaagccccagatatactgtgccttcaagagaccaaatgttcagagaacaaactaccagctgaacttcaggagctgcctggactctctcatcaatactggtcagctccttcggacaaggaagggtacagtggcgtgggcctgctttcccgccagtgcccactcaaagtttcttacggcataggtgagaccctattgatgcctaatgcctgaactcttcaaaaccaattgctaattctctatctctgccccacctcttgattgctttcccttttcttatagttttttatgctaattctgtttcatttctataggcgatgaggagcatgatcaggaaggccgggtgattgtggctgaatttgactcgtttgtgctggtaacagcatatgtacctaatgcaggccgaggtctggtacgactggagtaccggcagcgctgggatgaagcctttcgcaagttcctgaagggcctggcttcccgaaagccccttgtgctgtgtggagacctcaatgtggcacatgaagaaattgaccttcgcaaccccaaggggaacaaaaagaatgctggcttcacgccacaagagcgccaaggcttcggggaattactgcaggctgtgccactggctgacagctttaggcacctctaccccaacacaccctatgcctacaccttttggacttatatgatgaatgctcgatccaagaatgttggttggcgccttgattactttttgttgtcccactctctgttacctgcattgtgtgacagcaagatccgttccaaggccctcggcagtgatcactgtcctatcaccctatacctagcactgtgacaccacccctaaatcactttgagcctgggaaataagccccctcaactaccattccttctttaaacactcttcagagaaatctgcattctatttctcatgtataaaactaggaatcctccaaccaggctcctgtgatagagttcttttaagcccaagattttttatttgagggttttttgttttttaaaaaaaaattgaacaaagactactaatgactttgtttgaattatccacatgaaaataaagagccatagtttc

SEQ ID NO: 18 - protein, 318 aa, Homo sapiens [sequence omitted]

SEQ ID NO: 19 - DNA, 1161 bp, Homo sapiens
  cyclin-dependent kinase 3 (CDK3), cyclin- dependent protein kinase
ccacatggaagctggaggagcaaccgggagcgctgggctggggtgcaaattgcccagtgccttctgtttcccaggcagctctgtggccatggatatgttccagaaggtagagaagatcggagagggcacctatggggtggtgtacaaggccaagaacagggagacagggcagctggtggccctgaagaagatcagactggatttggagatggagggggtcccaagcactgccatcagggagatctcgctgctcaaggaactgaagcaccccaacatcgtccgactgctggacgtggtgcacaacgagaggaagctctatctggtgtttgagttcctcagccaggacctgaagaagtacatggactccaccccaggctcagagctccccctgcacctcatcaagagctacctcttccagctgctgcagggggtgagtttctgccactcacatcgggtcatccaccgagacctgaagccccagaacctgctcatcaatgagttgggtgccatcaagctggctgacttcggcctggctcgcgccttcggggtgcccctgcgcacctacacccatgaggtggtgacactgtggtatcgcgcccccgagattctcttgggcagcaagttctataccacagctgtggatatctggagcattggttgcatctttgcagagatggtgactcgaaaagccctgtttcctggtgactctgagattgaccagctctttcgtatctttcgtatgctggggacacccagcgaagacacatggcccggggtcacccagctgcctgactataagggcagcttccctaagtggaccaggaagggactggaagagattgtgcccaatctggagccagagggcagggacctgctcatgcaactcctgcagtatgaccccagccagcggatcacagccaagactgccctggcccacccgtacttctcatcccctgagccctccccagctgcccgccagtatgtgctgcagcgattccgccattgagaatgtcaaggccacactcagatcctttctcgagcagcagctgctgccccagctgcctcctacccattgccaagagaggatgcatctggggagagcaaagcactaaggaattcagcatcagcctgcagagggctgagtctgggttagtcctgccc

SEQ ID NO: 20 - protein, 305 aa, Homo sapiens
  cyclin-dependent kinase 3 (CDK3), cyclin- dependent protein kinase
MDMFQKVEKIGEGTYGVVYKAKNRETGQLVALKKIRLDLEMEGVPSTAIREISLLKELKHPNIVRLLDVVHNERKLYLVFEFLSQDLKKYMDSTPGSELPLHLIKSYLFQLLQGVSFCHSHRVIHRDLKPQNLLINELGAIKLADFGLARAFGVPLRTYTHEVVTLWYRAPEILLGSKFYTTAVDIWSIGCIFAEMVTRKALFPGDSEIDQLFRIFRMLGTPSEDTWPGVTQLPDYKGSFPKWTRKGLEEIVPNLEPEGRDLLMQLLQYDPSQRITAKTALAHPYFSSPEPSPAARQYVLQRFRH

SEQ ID NO: 21 - DNA, 2297 bp, Homo sapiens
  PIM1 oncogene serine threonine kinase
gcgccgcatcctggaggttgggatgctcttgtccaaaatcaactcgcttgcccacctgcgcgcccgcgcctgcaacgacctgcacgccaccaagctggcgccgggcaaggagaaggagcccctggagtcgcagtaccaggtgggcccgctactgggcagcggcggcttcggctcggtctactcaggcatccgcgtctccgacaacttgccggtggccatcaaacacgtggagaaggaccggatttccgactggggagagctgcctaatggcactcgagtgcccatggaagtggtcctgctgaagaaggtgagctcgggtttctccggcgtcattaggctcctggactggttcgagaggcccgacagtttcgtcctgatcctggagaggcccgagccggtgcaagatctcttcgacttcatcacggaaaggggagccctgcaagaggagctggcccgcagcttcttctggcaggtgctggaggccgtgcggcactgccacaactgcggggtgctccaccgcgacatcaaggacgaaaacatccttatcgacctcaatcgcggcgagctcaagctcatcgacttcgggtcgggggcgctgctcaaggacaccgtctacacggacttcgatgggacccgagtgtatagccctccagagtggatccgctaccatcgctaccatggcaggtcggcggcagtctggtccctggggatcctgctgtatgatatggtgtgtggagatattcctttcgagcatgacgaagagatcatcaggggccaggttttcttcaggcagagggtctcttcagaatgtcagcatctcattagatggtgcttggccctgagaccatcagataggccaaccttcgaagaaatccagaaccatccatggatgcaagatgttctcctgccccaggaaactgctgagatccacctccacagcctgtcgccggggcccagcaaatagcagcctttctggcaggtcctcccctctcttgtcagatgcccaggagggaagcttctgtctccagctttcccgagtaccagtgacacgtctcgccaagcaggacagtgcttgatacaggaacaacatttacaactcattccagatcccaggcccctggaggctgcctcccaacagtggggaagagtgactctccaggggtcctaggcctcaactcctcccatagatactctcttcttctcataggtgtccagcattgctggactctgaaatatcccgggggtggggggtgggggtgggtcagaaccctgccatggaactgtttccttcatcatgagttctgctgaatgccgcgatgggtcaggtaggggggaaacaggttgggatgggataggactagcaccattttaagtccctgtcacctcttccgactctttctgagtgccttctgtggggactccggctgtgctgggagaaatacttgaacttgcctcttttacctgctgcttctccaaaaatctgcctgggttttgttccctatttttctctcctgtcctccctcaccccctccttcatatgaaaggtgccatggaagaggctacagggccaaacgctgagccacctgcccttttttctcctcctttagtaaaactccgagtgaactggtcttcctttttggtttttacttaactgtttcaaagccaagacctcacacacacaaaaaatgcacaaacaatgcaatcaacagaaaagctgtaaatgtgtgtacagttggcatggtagtatacaaaaagattgtagtggatctaatttttaagaaattttgcctttaagttattttacctgtttttgtttcttgttttgaaagatgcgcattctaacctggaggtcaatgttatgtatttatttatttatttatttggttcccttcctannnnnnnnnnngctgctgccctagttttctttcctcctttcctcctctgacttggggaccttttgggggagggctgcgacgcttgctctgtttgtggggtgacgggactcaggcgggacagtgctgcagctccctggcttctgtggggcccctcacctacttacccaggtgggtcccggctctgtgggtgatggggaggggcattgctgactgtgtatataggataattatgaaaagcagttctggatggtgtgccttccagatcctctctggggctgtgttttgagcagcaggtagcctgctggttttatctgagtgaaatactgtacaggggaataaaagagatcttatttttttttttatacttggcgttttttgaataaaaaccttttgtcttaaaac

SEQ ID NO: 22 - protein, 313 aa, Homo sapiens [sequence omitted]

SEQ ID NO: 23 - DNA, 3178 bp, Homo sapiens
  CDC7 cell division cycle 7 (CDC7), CDC7 cell division cycle 7-like 1 (CDC7L1) protein serine threonine kinase
gatctcttggagacggcgacccaggcatctggggagccacagaagtcgtactcccttaaaccctgctttgctccccctgtggatgtaaccccttagctggcattttgcatctcaattggcttgtgatggaggcgtctttggggattcagatggatgagccaatggctttttctccccagcgtgaccggtttcaggctgaaggctctttaaaaaaaaacgagcagaattttaaacttgcaggtgttaaaaaagatattgagaagctttatgaagctgtaccacagcttagtaatgtgtttaagattgaggacaaaattggagaaggcactttcagctctgtttatttggccacagcacagttacaagtaggacctgaagagaaaattgctctaaaacacttgattccaacaagtcatcctataagaattgcagctgaacttcagtgcctaacagtggctggggggcaagataatgtcatgggagttaaatactgctttaggaagaatgatcatgtagttattgctatgccatatctggagcatgagtcgtttttggacattctgaattctctttcctttcaagaagtacgggaatatatgcttaatctgttcaaagctttgaaacgcattcatcagtttggtattgttcaccgtgatgttaagcccagcaattttttatataataggcgcctgaaaaagtatgccttggtagactttggtttggcccaaggaacccatgatacgaaaatagagcttcttaaatttgtccagtctgaagctcagcaggaaaggtgttcacaaaacaaatcccacataatcacaggaaacaagattccactgagtggcccagtacctaaggagctggatcagcagtccaccacaaaagcttctgttaaaagaccctacacaaatgcacaaattcagattaaacaaggaaaagacggaaaggagggatctgtaggcctttctgtccagcgctctgtttttggagaaagaaatttcaatatacacagctccatttcacatgagagccctgcagtgaaactcatgaagcagtcaaagactgtggatgtactgtctagaaagttagcaacaaaaaagaaggctatttctacgaaagttatgaatagtgctgtgatgaggaaaactgccagttcttgcccagctagcctgacctgtgactgctatgcaacagataaagtttgtagtatttgcctttcaaggcgtcagcaggttgcccctagggcaggtacaccaggattcagagcaccagaggtcttgacaaagtgccccaatcaaactacagcaattgacatgtggtctgcaggtgtcatatttctttctttgcttagtggacgatatccattttataaagcaagtgatgatttaactgctttggcccaaattatgacaattaggggatccagagaaactatccaagctgctaaaacttttgggaaatcaatattatgtagcaaagaagttccagcacaagacttgagaaaactctgtgagagactcaggggtatggattctagcactcccaagttaacaagtgatatacaagggcatgcttctcatcaaccagctatttcagagaagactgaccataaagcttcttgcctcgttcaaacacctccaggacaatactcagggaattcatttaaaaagggggatagtaatagctgtgagcattgttttgatgagtataataccaatttagaaggctggaatgaggtacctgatgaagcttatgacctgcttgataaacttctagatctaaatccagcttcaagaataacagcagaagaagctttgttgcatccattttttaaagatatgagcttgtgataatggatcttcatttaatgtttactgttatgaggtagaataaaaaagaatactttgtaatagccacaagttcttgtttagagaccagagcaggattaataatttattttaacattttagtgtttggtggcacattctaaaatatagattaagaatacttaaaatgcctgggatagttcttgggactaacaacatgatcttctttgagttaaacctacctaagtagattttaggtgggttcctattaggtcagatttttagcttccctaattacctttcactgacatacagaaaaaggagcagttttagttttaattaattaaaattaacagatgtgatgaggattaaatgaatcaaaagacttaatttgtagattcttttagagttatgagctaggtatagtttggggaaactcaacctggtgctggtgctcttaacaattttgtaaataaagaagataatttccttttctagaggtacatattaggccttttatgaacactaaaacaatgaggaaatgttggtcatggggcaaagtatcacttaaaattgaattcatccatttttaaaaaacacttcatgaaagcattctggtgtgaattgccatttttttcttactggcttctcaattttcttccttctctgcccctacctaaaacattctcctcggaaattacatggtgctgaccacaaagtttctggatgttttattaaatattgtacgtgtttacagttgggaatttaaaataatacatacactggttgataaagggaagctgcaggaccaaggtgaagattgatagtccaaatgcttttcttttttgagttgtatattttttcacaccatcttagatataattaggtagctgctgaaaggaaaagtgaatacagaattgacggtattattggagatttttcctctgcgtagagccatccagatctctgtatcctgttttgactaagtcttaggtgggttgggaagacagataatgaagtaggcaaagagaaaaggacccaagatagaggtttatattcagaaatggtatatatcaatgacagcatatcaaacttcctatgggaaaaagtctggtgggtggtcagctgacagatttcccatttagtagtcatagaatacagaaatagtttagggacatgtattcattttgttattttgagcattgataggtcagtatatctacctaatctgtttggtaagtataggatatataaaccattaccattgatctgtcttatgccataatcttaaaaaaaaattgaatgctcttgaatttgtatattcaataaagttatccttttataaaaaaaaa

SEQ ID NO: 24 - protein, 574 aa, Homo sapiens
  CDC7 cell division cycle 7 (CDC7), CDC7 cell division cycle 7-like 1 (CDC7L1) protein serine threonine kinase
MEASLGIQMDEPMAFSPQRDRFQAEGSLKKNEQNFKLAGVKKDIEKLYEAVPQLSNVFKIEDKIGEGTFSSVYLATAQLQVGPEEKIALKHLIPTSHPIRIAAELQCLTVAGGQDNVMGVKYCFRKNDHVVIAMPYLEHESFLDILNSLSFQEVREYMLNLFKALKRIHQFGIVHRDVKPSNFLYNRRLKKYALVDFGLAQGTHDTKIELLKFVQSEAQQERCSQNKSHIITGNKIPLSGPVPKELDQQSTTKASVKRPYTNAQIQIKQGKDGKEGSVGLSVQRSVFGERNFNIHSSISHESPAVKLMKQSKTVDVLSRKLATKKKAISTKVMNSAVMRKTASSCPASLTCDCYATDKVCSICLSRRQQVAPRAGTPGFRAPEVLTKCPNQTTAIDMWSAGVIFLSLLSGRYPFYKASDDLTALAQIMTIRGSRETIQAAKTFGKSILCSKEVPAQDLRKLCERLRGMDSSTPKLTSDIQGHASHQPAISEKTDHKASCLVQTPPGQYSGNSFKKGDSNSCEHCFDEYNTNLEGWNEVPDEAYDLLDKLLDLNPASRITAEEALLHPFFKDMSL

SEQ ID NO: 25 - DNA, 1427 bp, Homo sapiens
  cyclin-dependent kinase 7 (CDK7), kinase subunit of Cdk-activating kinase (CAK), kinase component of transcription factor complex TFIIH
tgggtgttggaggctttaaggtagctttaaattcgtgttgtcctgggagctcgcccttttcggctggagtcgggctttacggcgccggatggctctggacgtgaagtctcgggcaaagcgttatgagaagctggacttccttggggagggacagtttgccaccgtttacaaggccagagataagaataccaaccaaattgtcgccattaagaaaatcaaacttggacatagatcagaagctaaagatggtataaatagaaccgccttaagagagataaaattattacaggagctaagtcatccaaatataattggtctccttgatgcttttggacataaatctaatattagccttgtctttgattttatggaaactgatctagaggttataataaaggataatagtcttgtgctgacaccatcacacatcaaagcctacatgttgatgactcttcaaggattagaatatttacatcaacattggatcctacatagggatctgaaaccaaacaacttgttgctagatgaaaatggagttctaaaactggcagattttggcctggccaaatcttttgggagccccaatagagcttatacacatcaggttgtaaccaggtggtatcgggcccccgagttactatttggagctaggatgtatggtgtaggtgtggacatgtgggctgttggctgtatattagcagagttacttctaagggttccttttttgccaggagattcagaccttgatcagctaacaagaatatttgaaactttgggcacaccaactgaggaacagtggccggacatgtgtagtcttccagattatgtgacatttaagagtttccctggaatacctttgcatcacatcttcagtgcagcaggagacgacttactagatctcatacaaggcttattcttatttaatccatgtgctcgaattacggccacacaggcactgaaaatgaagtatttcagtaatcggccagggccaacacctggatgtcagctgccaagaccaaactgtccagtggaaaccttaaaggagcaatcaaatccagctttggcaataaaaaggaaaagaacagaggccttagaacaaggaggattgcccaagaaactaattttttaaagagaacactggacaacattttactactgagggaaatagccaaaaaggcaaataatggaaaaatagtaaacattaagtaaatgctgtagaagtgagtttgtaaatattctacacatgtaaaatatgtaaaactatgggttatttttattaaatgtattttaaaataaaaatttaattctggtttttctgattagagtcccaaagtgagaaaagttcaatactcttgaaatgtagaattgaaaatgcattagggaaaacttaataaaaattattaccagttatttggaaaaaaaaaaaa

SEQ ID NO: 26 - protein, 346 aa, Homo sapiens
  cyclin-dependent kinase 7 (CDK7), kinase subunit of Cdk-activating kinase (CAK), kinase component of transcription factor complex TFIIH
MALDVKSRAKRYEKLDFLGEGQFATVYKARDKNTNQIVAIKKIKLGHRSEAKDGINRTALREIKLLQELSHPNIIGLLDAFGHKSNISLVFDFMETDLEVIIKDNSLVLTPSHIKAYMLMTLQGLEYLHQHWILHRDLKPNNLLLDENGVLKLADFGLAKSFGSPNRAYTHQVVTRWYRAPELLFGARMYGVGVDMWAVGCILAELLLRVPFLPGDSDLDQLTRIFETLGTPTEEQWPDMCSLPDYVTFKSFPGIPLHHIFSAAGDDLLDLIQGLFLFNPCARITATQALKMKYFSNRPGPTPGCQLPRPNCPVETLKEQSNPALAIKRKRTEALEQGGLPKKLIF

SEQ ID NO: 27 - DNA, 2169 bp, Homo sapiens
  cytokine-inducible kinase (CNK) serine threonine kinase, proliferation-related kinase (PRK), polo-like kinase 3 (PLK3)
ccgcctccgagtgccttgcgcggacctgagctggagatgctggccgggctaccgacgtcagaccccgggcgcctcatcacggacccgcgcagcggccgcacctacctcaaaggccgcttgttgggcaaggggggcttcgcccgctgctacgaggccactgacacagagactggcagcgcctacgctgtcaaagtcatcccgcagagccgcgtcgccaagccgcatcagcgcgagaagatcctaaatgagattgagctgcaccgagacctgcagcaccgccacatcgtgcgtttttcgcaccactttgaggacgctgacaacatctacattttcttggagctctgcagccgaaagtccctggcccacatctggaaggcccggcacaccctgttggagccagaagtgcgctactacctgcggcagatcctttctggcctcaagtacttgcaccagcgcggcatcttgcaccgggacctcaagttgggaaattttttcatcactgagaacatggaactgaaggtgggggattttgggctggcagcccggttggagcctccggagcagaggaagaagaccatctgtggcacccccaactatgtggctccagaagtgctgctgagacagggccacggccctgaagcggatgtatggtcactgggctgtgtcatgtacacgctgctctgcgggagccctccctttgagacggctgacctgaaggagacgtaccgctgcatcaagcaggttcactacacgctgcctgccagcctctcactgcctgcccggcagctcctggccgccatccttcgggcctcaccccgagaccgcccctctattgaccagatcctgcgccatgacttctttaccaagggctacacccccgatcgactccctatcagcagctgcgtgacagtcccagacctgacaccccccaacccagctaggagtctgtttgccaaagttaccaagagcctctttggcagaaagaagaagagtaagaatcatgcccaggagagggatgaggtctccggtttggtgagcggcctcatgcgcacatccgttggccatcaggatgccaggccagaggctccagcagcttctggcccagcccctgtcagcctggtagagacagcacctgaagacagctcaccccgtgggacactggcaagcagtggagatggatttgaagaaggtctgactgtggccacagtagtggagtcagccctttgtgctctgagaaattgtatagctttcatgcccccagcggaacagaacccggcccccctggcccagccagagcctctggtgtgggtcagcaagtgggttgactactccaataagttcggctttgggtatcaactgtccagccgccgtgtggctgtgctcttcaacgatggcacacatatggccctgtcggccaacagaaagactgtgcactacaatcccaccagcacaaagcacttctccttctccgtgggtgctgtgccccgggccctgcagcctcagctgggtatcctgcggtacttcgcctcctacatggagcagcacctcatgaagggtggagatctgcccagtgtggaagaggtagaggtacctgctccgcccttgctgctgcagtgggtcaagacggatcaggctctcctcatgctgtttagtgatggcactgtccaggtgaacttctacggggaccacaccaagctgattctcagtggctgggagcccctccttgtgacttttgtggcccgaaatcgtagtgcttgtacttacctcgcttcccaccttcggcagctgggctgctctccagacctgcggcagcgactccgctatgctctgcgcctgctccgggaccgcagcccagcttaggacccaagccctgaaggcctgaggcctgtgcctgtcaggctctggcccttgcctttgtggccttcccccttcctttggtgcctcactgggggctttgggccgaatcccccagggaatcagggaccagctttactggagttgggggcggcttgtcttcgctggctcctaccccatctccaagataagcctgagccttagctcccagctagggggcgttatttatggaccacttttatttattgtcagacacttatttattgggatgtgagccccagggggcctcctcctaggataataaacaattttgca

SEQ ID NO: 28 - protein, 607 aa, Homo sapiens
  cytokine-inducible kinase (CNK) serine threonine kinase, proliferation-related kinase (PRK), polo-like kinase 3 (PLK3)
MLAGLPTSDPGRLITDPRSGRTYLKGRLLGKGGFARCYEATDTETGSAYAVKVIPQSRVAKPHQREKILNEIELHRDLQHRHIVRFSHHFEDADNIYIFLELCSRKSLAHIWKARHTLLEPEVRYYLRQILSGLKYLHQRGILHRDLKLGNFFITENMELKVGDFGLAARLEPPEQRKKTICGTPNYVAPEVLLRQGHGPEADVWSLGCVMYTLLCGSPPFETADLKETYRCIKQVHYTLPASLSLPARQLLAAILRASPRDRPSIDQILRHDFFTKGYTPDRLPISSCVTVPDLTPPNPARSLFAKVTKSLFGRKKKSKNHAQERDEVSGLVSGLMRTSVGHQDARPEAPAASGPAPVSLVETAPEDSSPRGTLASSGDGFEEGLTVATVVESALCALRNCIAFMPPAEQNPAPLAQPEPLVWVSKWVDYSNKFGFGYQLSSRRVAVLFNDGTHMALSANRKTVHYNPTSTKHFSFSVGAVPRALQPQLGILRYFASYMEQHLMKGGDLPSVEEVEVPAPPLLLQWVKTDQALLMLFSDGTVQVNFYGDHTKLILSGWEPLLVTFVARNRSACTYLASHLRQLGCSPDLRQRLRYALRLLRDRSPA

SEQ ID NO: 29 - DNA, 1321 bp, Homo sapiens
  potentially prenylated protein tyrosine phosphatase (PRL-3), protein tyrosine phosphatase type IVA, member 3, isoform 2, transcript variant 2 (PTP4A3)
tgactatccagctctgagagacgggagtttggagttgcccgctttactttggttgggttggggggggcggcgggctgttttgttccttttcttttttaagagttgggttttcttttttaattatccaaacagtgggcagcttcctcccccacacccaagtatttgcacaatatttgtgcggggtatgggggtgggtttttaaatctcgtttctcttggacaagcacagggatctcgttctcctcattttttgggggtgtgtggggacttctcaggtcgtgtccccagccttctctgcagtcccttctgccctgccgggcccgtcgggaggcgccatggctcggatgaaccgcccggccccggtggaggtgagctacaaacacatgcgcttcctcatcacccacaaccccaccaacgccacgctcagcaccttcattgaggacctgaagaagtacggggctaccactgtggtgcgtgtgtgtgaagtgacctatgacaaaacgccgctggagaaggatggcatcaccgttgtggactggccgtttgacgatggggcgcccccgcccggcaaggtagtggaagactggctgagcctggtgaaggccaagttctgtgaggcccccggcagctgcgtggctgtgcactgcgtggcgggcctgggccggaagcgccgcggagccatcaacagcaagcagctcacctacctggagaaataccggcccaaacagaggctgcggttcaaagacccacacacgcacaagacccggtgctgcgttatgtagctcaggaccttggctgggcctggtcgtcatgtaggtcaggaccttggctggacctggaggccctgcccagccctgctctgcccagcccagcaggggctccaggccttggctggccccacatcgccttttcctccccgacacctccgtgcacttgtgtccgaggagcgaggagcccctcgggccctgggtggcctctgggccctttctcctgtctccgccactccctctggcggcgctggccgtggctctgtctctctgaggtgggtcgggcgccctctgcccgccccctcccacaccagccaggctggtctcctctagcctgtttgttgtggggtgggggtatattttgtaaccactgggcccccagcccctcttttgcgaccccttgtcctgacctgttctcggcaccttaaattattagaccccggggcagtcaggtgctccggacacccgaaggcaataaaacaggagccgtgaaaaaaaaaaaaaaaaaaaaaaaaaaaaaaaaaaaaaaaaaaaaaaaaaaaaaaaaaaaaaaaa

SEQ ID NO: 30 - protein, 148 aa, Homo sapiens
  potentially prenylated protein tyrosine phosphatase (PRL-3), protein tyrosine phosphatase type IVA, member 3, isoform 2, transcript variant 2 (PTP4A3)
MARMNRPAPVEVSYKHMRFLITHNPTNATLSTFIEDLKKYGATTVVRVCEVTYDKTPLEKDGITVVDWPFDDGAPPPGKVVEDWLSLVKAKFCEAPGSCVAVHCVAGLGRKRRGAINSKQLTYLEKYRPKQRLRFKDPHTHKTRCCVM

SEQ ID NO: 31 - DNA, 3696 bp, Homo sapiens
  serine threonine kinase 2 (STK2, NEK4)
ggatcgctatggcagcggcgtcgtcgcgggccgggccccagcaatcccgcccgggcccggctgcctcaacagccgcccccactgccccctctcgggcatgaaccgagcttcttgttgccgcccgctgccctacccgccgctgccgccgcatcccgactctgggccagcgctgggaacatgcccctggccgcctactgctacctgcgggtcgtgggcaaggggagctatggagaggtgacgcttgtgaagcaccggcgggacggcaagcagtatgtcatcaaaaaactgaacctccgaaatgcctctagccgagagcggcgagctgctgaacaggaagcccagctcttgtctcagttgaagcatcccaacattgtcacctacaaggagtcatgggaaggaggagatggtctgctctacattgtcatgggcttctgtgaaggaggtgatttgtaccgaaagctcaaggagcagaaagggcagcttctgcctgagaatcaggtggtagagtggtttgtacagatcgccatggctttgcagtatttacatgaaaaacacatccttcatcgagatctgaaaactcaaaatgtcttcctaacaagaacaaacatcatcaaagtaggggacctaggaattgcccgagtgttagagaaccactgtgacatggctagcaccctcattggcacaccctactacatgagccctgaattgttctcaaacaaaccctacaactataagtctgatgtttgggctctaggatgctgtgtctatgaaatggccaccttgaagcatgctttcaatgcaaaagatatgaattctttagtttatcggattattgaaggaaagctgccaccaatgccaagagattacagcccagagctggcagaactgataagaacaatgctgagcaaaaggcctgaagaaaggccgtctgtgaggagcatcctgaggcagccttatataaagcggcaaatctccttctttttggaggccacaaagataaaaacctccaaaaataacattaaaaatggtgactctcaatccaagccttttgctacagtggtttctggagaggcagaatcaaatcatgaagtaatccacccccaaccactctcttctgagggctcccagacatatataatgggtgaaggcaaatgtttgtcccaggagaaacccagggcctctggtctcttgaagtcacctgccagtctgaaagcccatacctgcaaacaggacttgagcaataccacagaactagccacaatcagtagcgtaaatattgacatcttacctgcaaaagggagggattcagtgagtgatggctttgttcaggagaatcagccaagatatttggatgcctctaatgagttaggaggtatatgcagtatttctcaagtggaagaggagatgctgcaggacaacactaaatccagtgcccagcctgaaaacctgattcccatgtggtcctctgacattgtcactggggaaaagaatgaaccagtgaagcctctgcagcccctaatcaaagaacaaaagccaaaggaccagagtcttgccctgtcgcccaagctggagtgcagtggcacaatcttggctcacagcaacctccgcctcctgggttcaagtgattctccagcctcagcctcccgagtagctgggattacaggcgtgtgccaccacgcccaggatcaagttgctggtgaatgtattatagaaaaacagggcagaatccacccagatttacagccacacaactctgggtctgaaccttccctgtctcgacagcgacggcaaaagaggagagaacagactgagcacagaggggaaaagagacaggtccgcagagatctctttgctttccaagagtcgcctcctcgatttttgccttctcatcccattgttgggaaagtggatgtcacatcaacacaaaaagaggctgaaaaccaacgtagagtggtcactgggtctgtgagcagttcaaggagcagtgagatgtcatcatcaaaggatcgaccattatcagccagagagaggaggcgactaaagcagtcacaggaagaaatgtcctcttcaggcccttcagtgaggaaagcgtctctgagtgtagcagggccaggaaaaccccaggaagaagaccagcccttgcctgcccgacggctctcctctgactgcagcgtcactcaggaaaggaaacagattcattgtctgtctgaggatgagttaagttcttctacaagttcaactgataagtcagatggggattacggggaagggaaaggtcagacaaatgaaattaatgccttggtacaattgatgactcagaccctgaaactggattctaaagagagctgtgaagatgtcccggtagcaaacccagtgtcagaattcaaacttcatcggaaatatcgggacacactgatacttcatgggaaggttgcagaagaggcagaggaaatccattttaaagagctaccttcagctattatgccaggttctgaaaagatcaggagactagttgaagtcttgagaactgatgtaattcgtggcctgggagttcagcttttagagcaggtgtatgatcttttggaggaggaggatgaatttgatagagaggtacgtttgcgggagcacatgggtgaaaagtatacaacttacagtgtgaaagctcgccagttgaaattttttgaagaaaacatgaatttttgagcatttgtcctaatctgctgccagaattaaagacctatttttagaggattttggcttaaaaagcaagggcaaacagtcatttggaagccactcaccactgttttatatctcttttttatatctctttggcgtttccctacagaaaagaaattggacagaacagaataatatgaagcaggatcacaaaagaaaaaaaactttggctttcatattctctttgtgaggacaaatctgttgtttgtttgattactgtttactgagccttaatccaccaagtttatatttagaattttatttttttaaggtactaattaacttaaacacagagctataaaatgctggattgaaaattttatattgtaatgtagagataaaagcagtaggagaaacaaatgacataatatgtcgtcataattcctgctattgttaataaccttaaggagtagttgataaattataaaattttaaaaagtcaattcagttctagaaatagatttaaagaatatgaagttctatctagtacttgagcagctgtatttcttttctacacattgatggacttttaatattttattctcatttaatataaacctcatctagggtatatacaaattaaaactgagacacattggctttgtaaatcagtatgtttttacataatggttttgttagatttatttttccatcagtgaaaacatttcttaagcacaaatttcatttccatttaagcaatttgtaagcaaagtccaggtccatttagtttttggatatatttaatgtttgtctcctgaagtttgtcttcatgtactgtaagatattagttgtctttccatgttttaaatgtatgattatatagcacatattttattagttgtttaataagaggtaatacccatctaggaaagaaattttatgaagttaaatacaagtcttgaatagtacattttcacttctgtattcgagggactctaaaaataaatattgctccagaaa

SEQ ID NO: 32 - protein, 841 aa, Homo sapiens
  serine threonine kinase 2 (STK2, NEK4)
MPLAAYCYLRVVGKGSYGEVTLVKHRRDGKQYVIKKLNLRNASSRERRAAEQEAQLLSQLKHPNIVTYKESWEGGDGLLYIVMGFCEGGDLYRKLKEQKGQLLPENQVVEWFVQIAMALQYLHEKHILHRDLKTQNVFLTRTNIIKVGDLGIARVLENHCDMASTLIGTPYYMSPELFSNKPYNYKSDVWALGCCVYEMATLKHAFNAKDMNSLVYRIIEGKLPPMPRDYSPELAELIRTMLSKRPEERPSVRSILRQPYIKRQISFFLEATKIKTSKNNIKNGDSQSKPFATVVSGEAESNHEVIHPQPLSSEGSQTYIMGEGKCLSQEKPRASGLLKSPASLKAHTCKQDLSNTTELATISSVNIDILPAKGRDSVSDGFVQENQPRYLDASNELGGICSISQVEEEMLQDNTKSSAQPENLIPMWSSDIVTGEKNEPVKPLQPLIKEQKPKDQSLALSPKLECSGTILAHSNLRLLGSSDSPASASRVAGITGVCHHAQDQVAGECIIEKQGRIHPDLQPHNSGSEPSLSRQRRQKRREQTEHRGEKRQVRRDLFAFQESPPRFLPSHPIVGKVDVTSTQKEAENQRRVVTGSVSSSRSSEMSSSKDRPLSARERRRLKQSQEEMSSSGPSVRKASLSVAGPGKPQEEDQPLPARRLSSDCSVTQERKQIHCLSEDELSSSTSSTDKSDGDYGEGKGQTNEINALVQLMTQTLKLDSKESCEDVPVANPVSEFKLHRKYRDTLILHGKVAEEAEEIHFKELPSAIMPGSEKIRRLVEVLRTDVIRGLGVQLLEQVYDLLEEEDEFDREVRLREHMGEKYTTYSVKARQLKFFEENMNF

SEQ ID NO: 33 - DNA, 1513 bp, Homo sapiens
  serine threonine protein kinase NKIAMRE, mitogen-activated protein kinase/cyclin- dependent kinase-related protein kinase NKIATRE homologue
atggagatgtatgaaacccttggaaaagtgggagagggaagttacggaacagtcatgaaatgtaaacataagaatactgggcagatagtggccattaagatattttatgagagaccagaacaatctgtcaacaaaattgcgatgagagaaataaagtttctaaagcaatttcatcacgaaaacctggtcaatctgattgaagtttttagacagaaaaagaaaattcatttggtatttgaatttattgaccacacagtattagatgagttacaacattattgtcatggactagagagtaagcgacttagaaaatacctcttccagatccttcgagcaattgactatcttcacagtaataatatcattcatcgagatataaaacctgagaatattttagtatcccagtcaggaattactaagctctgtgattttggttttgcacgaacactagcagctcctggggacatttatacggactatgtggccacacgctggtatagagctcccgaattagtattaaaagatacttcttatggaaaacctgtggatatctgggctttgggctgtatgatcattgagatggccactggaaatccctatcttcctagtagttctgatttggatttactccataaaattgttttgaaagtgggcaatttgtcacctcacttgcagaatatcttttccaagagccccatttttgctggggtagttcttcctcaagttcaacaccccaaaaatgcaagaaaaaaatatccaaagcttaatggattgttggcagatatagttcatgcttgtttacaaattgatcctgctgacaggatatcatctagtgatcttttgcatcatgagtattttactagagatggatttattgaaaaattcatgccagaactgaaagctaaattactgcaggaagcaaaagtcaattcattaataaagccaaaagagagttctaaagaaaatgaactcaggaaagatgaaagaaaaacagtttataccaatacactgctaagtagttcagttttgggagaggaaatagaaaaagagaaaaagcccaaggagatcaaagtcagagttattaaagtcaaaggaggaagaggagatatctcagaaccaaaaaagaaagagtatgaaggtggacttggtcaacaggatgcaaatgaaaatgttcatcctatgtctccagatacaaaacttgtaaccattgaaccaccaaaccctatcaatcccagcactaactgtaatggcttgaaagaaaatccacattgcggaggttctgtaacaatgccacccatcaatctaactaacagtaatttgatggctgcaaatctcagttcaaatctctttcaccccagtgtgaggtgagctgtaacagagaagaaacctaaataatacaacattcctgtataatggtatttcaaagaatcgtgttcatagtgtctgtatgtaaactgaacttgaagaaaatatattgaaattaaagctgtataatgggccaaaaaaaaaaaaa

SEQ ID NO: 34 - protein, 455 aa, Homo sapiens
  serine threonine protein kinase NKIAMRE, mitogen-activated protein kinase/cyclin- dependent kinase-related protein kinase NKIATRE homologue
MEMYETLGKVGEGSYGTVMKCKHKNTGQIVAIKIFYERPEQSVNKIAMREIKFLKQFHHENLVNLIEVFRQKKKIHLVFEFIDHTVLDELQHYCHGLESKRLRKYLFQILRAIDYLHSNNIIHRDIKPENILVSQSGITKLCDFGFARTLAAPGDIYTDYVATRWYRAPELVLKDTSYGKPVDIWALGCMIIEMATGNPYLPSSSDLDLLHKIVLKVGNLSPHLQNIFSKSPIFAGVVLPQVQHPKNARKKYPKLNGLLADIVHACLQIDPADRISSSDLLHHEYFTRDGFIEKFMPELKAKLLQEAKVNSLIKPKESSKENELRKDERKTVYTNTLLSSSVLGEEIEKEKKPKEIKVRVIKVKGGRGDISEPKKKEYEGGLGQQDANENVHPMSPDTKLVTIEPPNPINPSTNCNGLKENPHCGGSVTMPPINLTNSNLMAANLSSNLFHPSVR

SEQ ID NO: 35 - DNA, 3504 bp, Homo sapiens
  HBO1 histone acetyltransferase, MYST histone acetyltransferase 2 (MYST2)
gccgctgcccgaatcggaaccgtcgggccgcagccgccggcaatgccgcgaaggaagaggaatgcaggcagtagttcagatggaaccgaagattccgatttttctacagatctcgagcacacagacagttcagaaagtgatggcacatcccgacgatctgctcgagtcacccgctcctcagccaggctaagccagagttctcaagattccagtcctgttcgaaatctgcagtcttttggcactgaggagcctgcttactctaccagaagagtgacccgtagtcagcagcagcctaccccagtgacaccgaaaaaataccctcttcggcagactcgttcatctggttcagaaactgagcaagtggttgatttttcagatagagaaactaaaaatacagctgatcatgatgagtcaccgcctcgaactccaactggaaatgcgccttcttctgagtctgacatagatatctccagccccaatgtatctcacgatgagagcattgccaaggacatgtccctgaaggactcaggcagtgatctctctcatcgccccaagcgccgtcgcttccatgaaagctacaacttcaatatgaagtgtcctacaccaggctgtaactctctaggacaccttacaggaaaacatgagagacatttctccatctcaggatgcccactgtatcataacctctcagctgacgaatgcaaggtgagagcacagagccgggataagcagatagaagaaaggatgctgtctcacaggcaagatgacaacaacaggcatgcaaccaggcaccaggcaccaacggagaggcagcttcgatataaggaaaaagtggctgaactcaggaagaaaagaaattctggactgagcaaagaacagaaagagaaatatatggaacacagacagacctatgggaacacacgggaacctcttttagaaaacctgacaagcgagtatgacttggatcttttccgaagagcacaagcccgggcttcagaggatttggagaagttaaggctgcaaggccaaatcacagagggaagcaacatgattaaaacaattgcttttggccgctatgagcttgatacctggtatcattctccatatcctgaagaatatgcacggctgggacgtctctatatgtgtgaattctgtttaaaatatatgaagagccaaacgatactccgccggcacatggccaaatgtgtgtggaaacacccacctggtgatgagatatatcgcaaaggttcaatctctgtgtttgaagtggatggcaagaaaaacaagatctactgccaaaacctgtgcctgttggccaaactttttctggaccacaagacattatattatgatgtggagcccttcctgttctatgttatgacagaggcggacaacactggctgtcacctgattggatatttttctaaggaaaagaattcattcctcaactacaacgtctcctgtatccttactatgcctcagtacatgagacagggctatggcaagatgcttattgatttcagttatttgctttccaaagtcgaagaaaaagttggctccccagaacgtccactctcagatctggggcttataagctatcgcagttactggaaagaagtacttctccgctacctgcataattttcaaggcaaagagatttctatcaaagaaatcagtcaggagacggctgtgaatcctgtggacattgtcagcactctgcaagcccttcagatgctcaaatactggaagggaaaacacctagttttaaagagacaggacctgattgatgagtggatagccaaagaggccaaaaggtccaactccaataaaaccatggatcccagctgcttaaaatggacccctcccaagggcacttaaagtgacctgtcattccgagccagcgaaccccagcagtaggaatccgtaccctagggatctgtctgtcatttctctgttgctcttgtgattggcaagtacagtatcctttgggaaggccatccccctcaggactgtcctggctccgacctttgtgtacactgcagacgctggttctgaggaactgttgtttcggcctcagtgaggttgcctggatgggatctgtattagacttgagtgcaggtctctcagcactgacccaaggagttctgttatggtactgtacctgtccagtcactggttctctcctcatgtcctctcgccccatgaggttgtgttgtgtcttctaagcgtggtactagtgcttgccacctggtcaccagacctccaaatatggctgccaccaccaggacctttccagttactccttatatgtgtgttctatggaggggcagggaaaaggtggcacttgtgagtgtgtgtggattggcagggggtccattcactttgggttccatcttgctttaaatttcttcattttgattaagagacctctttttgatctgtattgggctaaccagagccaaatacttttgaagagtttcccagggactagtcatggtaatagcatataattgatctgaatgagatggagagaagaatgaaggggtggtggttctgggtttgatttgagttcacctgtgggcagtgggcagtgggcagtgtcttggtgaaagggaacggatactactttttgcctcaccgtaaagtactcactagtaaatatttccttctctctttactcccactttttacgtttgcaggtgccaaagtaatgtccacttttccctttcatgctgcatattaactggttaattatactgcagaaaccttttcacctccactagtctgatacagtacatctgtacttccatataccttgcactgattttgtctgagtgccctgggagaagtagaaaatgattgaaagtgacttccgtatctcagcccatgactcagcaaggcagaatggccacccctgccaaagtttgcttctcttttcaacagtgcctcaccctccctctaggattaaagtgcttctgcccttccacgaactcctcctccatttcctttttgggatttgtcaccatccttctattctctggtcttctatttttggtgttgttcaagtgaaggaagagatgttccctctaatttctctctagcccattataacctgctatcttggggcaacttttgatgtatgacatgtcacccttcccaacttggtctcctccaacatgctgtcttcatgtggagccctcaccacaatccctgactccggtcatttgtgcctttctcttgtcatctctgtacactacttatattcactgtgggttgggggagctaattttaagcatgttcagtggcagctcccctccagtttcagtgtcactgttaaaatttatcaaaaagcaacttcactaggggttttcttaagggataaaggccttttacagaagctaaacccttccccacatgtggtagaatgtgctcttctatatctactcctcaataaagcatgttctctgctcaaaaaaaaaaaaaaaaaaaaaaaaaaaaaaaaaa

SEQ ID NO: 36 - protein, 611 aa, Homo sapiens
  HBO1 histone acetyltransferase, MYST histone acetyltransferase 2 (MYST2)
MPRRKRNAGSSSDGTEDSDFSTDLEHTDSSESDGTSRRSARVTRSSARLSQSSQDSSPVRNLQSFGTEEPAYSTRRVTRSQQQPTPVTPKKYPLRQTRSSGSETEQVVDFSDRETKNTADHDESPPRTPTGNAPSSESDIDISSPNVSHDESIAKDMSLKDSGSDLSHRPKRRRFHESYNFNMKCPTPGCNSLGHLTGKHERHFSISGCPLYHNLSADECKVRAQSRDKQIEERMLSHRQDDNNRHATRHQAPTERQLRYKEKVAELRKKRNSGLSKEQKEKYMEHRQTYGNTREPLLENLTSEYDLDLFRRAQARASEDLEKLRLQGQITEGSNMIKTIAFGRYELDTWYHSPYPEEYARLGRLYMCEFCLKYMKSQTILRRHMAKCVWKHPPGDEIYRKGSISVFEVDGKKNKIYCQNLCLLAKLFLDHKTLYYDVEPFLFYVMTEADNTGCHLIGYFSKEKNSFLNYNVSCILTMPQYMRQGYGKMLIDFSYLLSKVEEKVGSPERPLSDLGLISYRSYWKEVLLRYLHNFQGKEISIKEISQETAVNPVDIVSTLQALQMLKYWKGKHLVLKRQDLIDEWIAKEAKRSNSNKTMDPSCLKWTPPKGT

SEQ ID NO: 37 - DNA, 21 bp, Artificial Sequence
  Description of Artificial SequenceCK2-specific siRNA molecule
aacattgaattagatccacgt

SEQ ID NO: 38 - DNA, 21 bp, Artificial Sequence
  Description of Artificial SequencePIM1- specific siRNA molecule
aaaactccgagtgaactggtc

SEQ ID NO: 39 - DNA, 21 bp, Artificial Sequence
  Description of Artificial SequenceHBO1- specific siRNA molecule
aactgagcaagtggttgattt

SEQ ID NO: 40 - protein, 409 aa, Homo sapiens
  CDC7 cell division cycle 7 (CDC7), CDC7 cell division cycle 7-like 1 (CDC7L1) protein serine threonine kinase
MEASLGIQMDEPMAFSPQRDRFQAEGSLKKNEQNFKLAGVKKDIEKLYEAVPQLSNVFKIEDKIGEGTFSSVYLATAQLQVGPEEKIALKHLIPTSHPIRIAAELQCLTVAGGQDNVMGVKYCFRKNDHVVIAMPYLEHESFLDILNSLSFQEVREYMLNLFKALKRIHQFGIVHRDVKPSNFLYNRRLKKYALVDFGLAQGTHDTKIELLKFVQSEAQQERCSQNKSHIITGNKIPLSGPVPKELDQQSTTKASVKRPYTNAQIQIKQGKDGKEGSVGLSVQRSVFGERNFNIHSSISHESPAVKLMKQSKTVDVLSRKLATKKKAISTKVMNSAVMRKTASSCPASLTCDCYATDKVCSICLSRRQQVAPRAGTPGFRAPEVLTKCPNQTTAIDMWSAGVIFLSLLS

SEQ ID NO: 41 - protein, 314 aa, Saccharomyces cerevisiae
  CDC7
MTSKTKNIDDIPPEIKEEMIQLYHDLPGIENEYKLIDKIGEGTFSSVYKAKDITGKITKKFASHFWNYGSNYVALKKIYVTSSPQRIYNELNLLYIMTGSSRVAPLCDAKRVRDQVIAVLPYYPHEEFRTFYRDLPIKGIKKYIWELLRALKFVHSKGIIHRDIKPTNFLFNLELGRGVLVDFGLAEAQMDYKSMISSQNDYDNYANTNHDGGYSMRNHEQFCPCIMRNQYSPNSHNQTPPMVTIQNGKVVHLNNVNGVDLTKGYPKNETRRIKRANRAGTRGFRAPEVLMKCGAQSTKIDIWSVGVILLSLLG

SEQ ID NO: 42 - protein, 294 aa, Artificial Sequence
  Description of Artificial Sequenceprotein kinase consensus sequence
YELLEKLGEGSFGKVYKAKHKDKTGKIVAVKILKKEKESIKEKRFLREIQILKRLSHPNIVRLIGVFEDTDDHLYLVMEYMEGGDLFDYLRRNGGPLSEKEAKKIALQILRGLEYLHSNGIVHRDLKPENILLDENDGTVKIADFGLARLLESSSKLTTFVGTPWYMMAPEVILEGRGYSSKVDVWSLGVILYELLTGGPLFPGADLPAFTGGDEVDQLIIFVLKLPFSDELPKTRIDPLEELFRIIKRPGLRLPLPSNCSEELKDLLKKCLNKDPSKRPGSATAKEILNHPWF

SEQ ID NO: 43 - protein, 253 aa, Homo sapiens
  cytokine-inducible kinase (CNK) serine threonine kinase, proliferation-related kinase (PRK), polo-like kinase 3 (PLK3)
YLKGRLLGKGGFARCYEATDTETGSAYAVKVIPQSRVAKPHQREKILNEIELHRDLQHRHIVRFSHHFEDADNIYIFLELCSRKSLAHIWKARHTLLEPEVRYYLRQILSGLKYLHQRGILHRDLKLGNFFITENMELKVGDFGLAARLEPPEQRKKTICGTPNYVAPEVLLRQGHGPEADVWSLGCVMYTLLCGSPPFETADLKETYRCIKQVHYTLPASLSLPARQLLAAILRASPRDRPSIDQILRHDFF

SEQ ID NO: 44 - protein, 5 aa, Artificial Sequence
  Description of Artificial Sequenceconsensus peptide
HRDLK

SEQ ID NO: 45 - protein, 5 aa, Artificial Sequence
  Description of Artificial Sequenceconsensus peptide
DFGLA

SEQ ID NO: 46 - protein, 4 aa, Artificial Sequence
  Description of Artificial Sequenceconsensus peptide
APEV

SEQ ID NO: 47 - protein, 6 aa, Artificial Sequence
  Description of Artificial Sequenceconsensus peptide
DVWSLG

SEQ ID NO: 48 - protein, 256 aa, Homo sapiens
  serine threonine kinase 2 (STK2, NEK4)
YCYLRVVGKGSYGEVTLVKHRRDGKQYVIKKLNLRNASSRERRAAEQEAQLLSQLKHPNIVTYKESWEGGDGLLYIVMGFCEGGDLYRKLKEQKGQLLPENQVVEWFVQIAMALQYLHEKHILHRDLKTQNVFLTRTNIIKVGDLGIARVLENHCDMASTLIGTPYYMSPELFSNKPYNYKSDVWALGCCVYEMATLKHAFNAKDMNSLVYRIIEGKLPPMPRDYSPELAELIRTMLSKRPEERPSVRSILRQPYI

SEQ ID NO: 49 - protein, 5 aa, Artificial Sequence
  Description of Artificial Sequenceconsensus peptide
HPNIV

SEQ ID NO: 50 - protein, 5 aa, Artificial Sequence
  Description of Artificial Sequenceconsensus peptide
EGGDL

SEQ ID NO: 51 - protein, 294 aa, Artificial Sequence; identical to SEQ ID NO: 42
  Description of Artificial Sequenceprotein kinase consensus sequence

SEQ ID NO: 52 - protein, 286 aa, Homo sapiens
  serine threonine protein kinase casein kinase 2, alpha 1 subunit isoform a, transcript variant 2 (CK2, CK2alpha), CK2 catalytic subunit alpha
YQLVRKLGRGKYSEVFEAINITNNEKVVVKILKPVKKKKIKREIKILENLRGGPNIITLADIVKDPVSRTPALVFEHVNNTDFKQLYQTLTDYDIRFYMYEILKALDYCHSMGIMHRDVKPHNVMIDHEHRKLRLIDWGLAEFYHPGQEYNVRVASRYFKGPELLVDYQMYDYSLDMWSLGCMLASMIFRKEPFFHGHDNYDQLVRIAKVLGTEDLYDYIDKYNIELDPRFNDILGRHSRKRWERFVHSENQHLVSPEALDFLDKLLRYDHQSRLTAREAMEHPYF

SEQ ID NO: 53 - protein, 5 aa, Artificial Sequence
  Description of Artificial Sequenceconsensus peptide
VKILK

SEQ ID NO: 54 - protein, 4 aa, Artificial Sequence
  Description of Artificial Sequenceconsensus peptide
WSLG

SEQ ID NO: 55 - protein, 298 aa, Homo sapiens
  cyclin-dependent kinase 2 (CDK2)
MENFQKVEKIGEGTYGVVYKARNKLTGEVVALKKIRLDTETEGVPSTAIREISLLKELNHPNIVKLLDVIHTENKLYLVFEFLHQDLKKFMDASALTGIPLPLIKSYLFQLLQGLAFCHSHRVLHRDLKPQNLLINTEGAIKLADFGLARAFGVPVRTYTHEVVTLWYRAPEILLGCKYYSTAVDIWSLGCIFAEMVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRL

SEQ ID NO: 56 - protein, 111 aa, Artificial Sequence
  Description of Artificial SequenceXeroderma pigmentosum complementation group XPG N- terminal domain (XPG_N) consensus sequence
MGIKGLLPILKPVAPEAIRSVSIEALEGYYKVLAIDASIWLYQFLKAVRDQLGNNLENEEGETTSHLMGLFSRLCRLLDFGIKPIFVFDGGAPNDLKAETLQKRSARRQEA

SEQ ID NO: 57 - protein, 107 aa, Artificial Sequence
  flap structure-specific endonuclease 1 (FEN1) 5′-3′ exonuclease
MGIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEA

SEQ ID NO: 58 - protein, 5 aa, Artificial Sequence
  Description of Artificial Sequenceconsensus peptide
AIDAS

SEQ ID NO: 59 - protein, 4 aa, Artificial Sequence
  Description of Artificial Sequenceconsensus peptide
YQFL

SEQ ID NO: 60 - protein, 12 aa, Artificial Sequence
  Description of Artificial Sequenceconsensus peptide
NEEGETTSHLMG

SEQ ID NO: 61 - protein, 4 aa, Artificial Sequence
  Description of Artificial Sequenceconsensus peptide
GIKP

SEQ ID NO: 62 - protein, 4 aa, Artificial Sequence
  Description of Artificial Sequenceconsensus peptide
VFDG

SEQ ID NO: 63 - protein, 104 aa, Artificial Sequence
  Description of Artificial SequenceXeroderma pigmentosum complementation group XPG I-region domain (XPG_I) consensus sequence
RLMGIPYIVAPGVEAEAQCAYLEKKGLVDGIITEDSDVLLFGAPRLLRNLTLSGKKSGPSITSLKVEIEEIDLESLLRELGLGKLSREQLIDLAILLGCDYTEG

SEQ ID NO: 64 - protein, 92 aa, Homo sapiens
  flap structure-specific endonuclease 1 (FEN1) 5′-3′ exonuclease
SLMGIPYLDAPSEAEASCAALVKAGKVYAAATEDMDCLTFGSPVLMRHLTASEAKKLPIQEFHLSRILQELGLNQEQFVDLCILLGSDYCES

SEQ ID NO: 65 - protein, 6 aa, Artificial Sequence
  Description of Artificial Sequenceconsensus peptide
LMGIPY

SEQ ID NO: 66 - protein, 4 aa, Artificial Sequence
  Description of Artificial Sequenceconsensus peptide
EAEA

SEQ ID NO: 67 - protein, 4 aa, Artificial Sequence
  Description of Artificial Sequenceconsensus peptide
ELGL

SEQ ID NO: 68 - protein, 4 aa, Artificial Sequence
  Description of Artificial Sequenceconsensus peptide
ILLG

SEQ ID NO: 69 - protein, 261 aa, Homo sapiens
  HBO1 histone acetyltransferase, MYST histone acetyltransferase 2 (MYST2)
YHSPYPEEYARLGRLYMCEFCLKYMKSQTILRRHMAKCVWKHPPGDEIYRKGSISVFEVDGKKNKIYCQNLCLLAKLFLDHKTLYYDVEPFLFYVMTEADNTGCHLIGYFSKEKNSFLNYNVSCILTMPQYMRQGYGKMLIDFSYLLSKVEEKVGSPERPLSDLGLISYRSYWKEVLLRYLHNFQGKEISIKEISQETAVNPVDIVSTLQALQMLKYWKGKHLVLKRQDLIDEWIAKEAKRSNSNKTMDPSCLKWTPPKGT

SEQ ID NO: 70 - protein, 265 aa, Saccharomyces cerevisiae
  Esa1
YFSPYPIELTDEDFIYIDDFTLQYFGSKKQYERYRKKCTLRHPPGNEIYRDDYVSFFEIDGRKQRTWCRNLCLLSKLFLDHKTLYYDVDPFLFYCMTRRDELGHHLVGYFSKEKESADGYNVACILTLPQYQRMGYGKLLIEFSYELSKKENKVGSPEKPLSDLGLLSYRAYWSDTLITLLVEHQKEITIDEISSMTSMTTTDILHTAKTLNILRYYKGQHIIFLNEDILDRYNRLKAKKRRTIDPNRLIWKPPVFTASQLRFAW

SEQ ID NO: 71 - protein, 253 aa, Homo sapiens
  PIM1 oncogene serine threonine kinase
YQVGPLLGSGGFGSVYSGIRVSDNLPVAIKHVEKDRISDWGELPNGTRVPMEVVLLKKVSSGFSGVIRLLDWFERPDSFVLILERPEPVQDLFDFITERGALQEELARSFFWQVLEAVRHCHNCGVLHRDIKDENILIDLNRGELKLIDFGSGALLKDTVYTDFDGTRVYSPPEWIRYHRYHGRSAAVWSLGILLYDMVCGDIPFEHDEEIIRGQVFFRQRVSSECQHLIRWCLALRPSDRPTFEEIQNHPWM

SEQ ID NO: 72 - protein, 4 aa, Artificial Sequence
  Description of Artificial Sequenceconsensus peptide
DLFD

SEQ ID NO: 73 - protein, 4 aa, Artificial Sequence
  Description of Artificial Sequenceconsensus peptide
ENIL

SEQ ID NO: 74 - protein, 5 aa, Artificial Sequence
  Description of Artificial Sequenceconsensus peptide
VWSLG

SEQ ID NO: 75 - protein, 4 aa, Artificial Sequence
  Description of Artificial Sequenceconsensus peptide
NHPW

SEQ ID NO: 76 - DNA, 13 bp, Artificial Sequence
  Description of Artificial Sequence5′-end 32P-labeled oligonucleotide primer
cactgactgtatg

SEQ ID NO: 77 - DNA, 30 bp, Artificial Sequence
  Description of Combined DNA/RNA Moleculeoligonucleotide template
ctcgtcagcatcttcaucatacagtcagtg

SEQ ID NO: 78 - protein, 200 aa, Artificial Sequence
  Description of Artificial Sequencepoly Gly flexible linker
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG